SEQUENCE LISTING (57 sequences)

SEQ ID NO: 1 - DNA, 7502 bp, Chlamydia trachomatis
ggatccgtaagttagacgaaattttgtctttgcgcacagacgatctattttttgcatccaatcagatttcctttcgcattaaaaaaagacagaataaagaaaccaaaattctaatcacatttcctatcagcttaatggaagagttgcaaaaatacacttgtgggagaaatgggagagtatttgtttctaaaatagggattcctgtaacaacaagtcaggttgcgcataattttaggcttgcagagttccatagtgctatgaaaataaaaattactcccagagtacttcgtgcaagcgctttgattcatttaaagcaaataggattaaaagatgaggaaatcatgcgtatttcctgtctttcatcgagacaaagtgtgtgttcttattgttctggggaagaggtaattcctctagtacaaacacccacaatattgtgatataattaaaattatattcatattctgttgccagaaaaaacacctttaggctatattagagccatcttctttgaagcgttgtcttctcgagaagatttatcgtacgcaaatatcatctttgcggttgcgtgtcctgtgaccttcattatgtcggagtctgagcaccctaggcgtttgtactccgtcacagcggttgctcgaagcacgtgcggggttattttaaaagggattgcagcttgtagtcctgcttgagagaacgtgcgggcgatttgccttaaccccaccatttttccggagcgagttacgaagacaaaacctcttcgttgaccgatgtactcttgtagaaagtgcataaacttctgaggataagttataataatcctcttttctgtctgacggttcttaagctgggagaaagaaatggtagcttgttggaaacaaatctgactaatctccaagcttaagacttcagaggagcgtttacctccttggagcattgtctgggcgatcaaccaatcccgggcattgattttttttagctcttttaggaaggatgctgtttgcaaactgttcatcgcatccgtttttactatttccctggttttaaaaaatgttcgactattttcttgtttagaaggttgcgctatagcgactattccttgagtcatcctgtttaggaatcttgttaaggaaatatagcttgctgctcgaacttgtttagtaccttcggtccaagaagtcttggcagaggaaacttttttaatcgcatctaggattagattatgatttaaaagggaaaactcttgcagattcatatccaaggacaatagaccaatcttttctaaagacaaaaaagatcctcgatatgatctacaagtatgtttgttgagtgatgcggtccaatgcataataacttcgaataaggagaagcttttcatgcgtttccaataggattcttggcgaatttttaaaacttcctgataagacttttcactatattctaacgacatttcttgctgcaaagataaaatccctttacccatgaaatccctcgtgatataacctatccgtaaaatgtcctgattagtgaaataatcaggttgttaacaggatagcacgctcggtatttttttatataaacatgaaaactcgttccgaaatagaaaatcgcatgcaagatatcgagtatgcgttgttaggtaaagctctgatatttgaagactctactgagtatattctgaggcagcttgctaattatgagtttaagtgttctcatcataaaaacatattcatagtatttaaacacttaaaagacaatggattacctataactgtagactcggcttgggaagagcttttgcggcgtcgtatcaaagatatggacaaatcgtatctcgggttaatgttgcatgatgctttatcaaatgacaagcttagatccgtttctcatacggttttcctcgatgatttgagcgtgtgtagcgctgaagaaaatttgagtaatttcattttccgctcgtttaatgagtacaatgaaaatccattgcgtagatctccgtttctattgcttgagcgtataaagggaaggcttgatagtgctatagcaaagactttttctattcgcagcgctagaggccggtctatttatgatatattctcacagtcagaaattggagtgctggctcgtataaaaaaaagacgagtagcgttctctgagaatcaaaattctttctttgatggcttcccaacaggatacaaggatattgatgataaaggagttatcttagctaaaggtaatttcgtgattatagcagctagaccatctatagggaaaacagctttagctatagacatggcgataaatcttgcggttactcaacagcgtagagttggtttcctatctctagaaatgagcgcaggtcaaattgttgagcggattattgctaatttaacaggaatatctggtgaaaaattacaaagaggggatctctctaaagaagaattattccgagtagaagaagctggagaaacggttagagaatcacatttttatatctgcagtgatagtcagtataagcttaacttaatcgcgaatcagatccggttgctgagaaaagaagatcgagtagacgtaatatttatcgattacttgcagttgatcaactcatcggttggagaaaatcgtcaaaatgaaatagcagatatatctagaaccttaagaggtttagcctcagagctaaacattcctatagtttgtttatcccaactatctagaaaagttgaggatagagcaaataaagttcccatgctttcagatttgcgagacagcggtcaaatagagcaagacgcagatgtgattttgtttatcaataggaaggaatcgtcttctaattgtgagataactgttgggaaaaatagacatggatcggttttctcttcggtattacatttcgatccaaaaattagtaaattctccgctattaaaaaagtatggtaaattatagtaactgccacttcatcaaaagtcctatccaccttgaaaatcagaagtttggaagaagacctggtcaatctattaagatatctcccaaattggctcaaaatgggatggtagaagttataggtcttgattttctttcatctcattaccatgcattagcagctatccaaagattactgaccgcaacgaattacaaggggaacacaaaaggggttgttttatccagagaatcaaatagttttcaatttgaaggatggataccaagaatccgttttacaaaaactgaattcttagaggcttatggagttaagcggtataaaacatccagaaataagtatgagtttagtggaaaagaagctgaaactgctttagaagccttataccatttaggacatcaaccgtttttaatagtggcaactagaactcgatggactaatggaacacaaatagtagaccgttaccaaactctttctccgatcattaggatttacgaaggatgggaaggtttaactgacgaagaaaatatagatatagacttaacaccttttaattcaccacctacacggaaacataaagggttcgttgtagagccatgtcctatcttggtagatcaaatagaatcctactttgtaatcaagcctgcaaatgtataccaagaaataaaaatgcgtttcccaaatgcatcaaagtatgcttacacatttatcgactgggtgattacagcagctgcgaaaaagagacgaaaattaactaaggataattcttggccagaaaacttgttattaaacgttaacgttaaaagtcttgcatatattttaaggatgaatcggtacatctgtacaaggaactggaaaaaaatcgagttagctatcgataaatgtatagaaatcgccattcagcttggctggttatctagaagaaaacgcattgaatttctggattcttctaaactctctaaaaaagaaattctatatctaaataaagagcgctttgaagaaataactaagaaatctaaagaacaaatggaacaattagaacaagaatctattaattaatagcaagcttgaaactaaaaacctaatttatttaaagctcaaaataaaaaagagttttaaaatgggaaattctggtttttatttgtataacactgaaaactgcgtctttgctgataatatcaaagttgggcaaatgacagagccgctcaaggaccagcaaataatccttgggacaacatcaacacctgtcgcagccaaaatgacagcttctgatggaatatctttaacagtctccaataattcatcaaccaatgcttctattacaattggtttggatgcggaaaaagcttaccagcttattctagaaaagttgggagatcaaattcttgatggaattgctgatactattgttgatagtacagtccaagatattttagacaaaatcaaaacagacccttctctaggtttgttgaaagcttttaacaactttccaatcactaataaaattcaatgcaacgggttattcactcccagtaacattgaaactttattaggaggaactgaaataggaaaattcacagtcacacccaaaagctctgggagcatgttcttagtctcagcagatattattgcatcaagaatggaaggcggcgttgttctagctttggtacgagaaggtgattctaagccctgcgcgattagttatggatactcatcaggcattcctaatttatgtagtctaagaaccagtattactaatacaggattgactccgacaacgtattcattacgtgtaggcggtttagaaagcggtgtggtatgggttaatgccctttctaatggcaatgatattttaggaataacaaatacttctaatgtatcttttttagaggtaatacctcaaacaaacgcttaaacaatttttattggatttttcttataggttttatatttagagaaaacagttcgaattacggggtttgttatgcaaaataaaagaaaagtgagggacgattttattaaaattgttaaagatgtgaaaaaagatttccccgaattagacctaaaaatacgagtaaacaaggaaaaagtaactttcttaaattctcccttagaactctaccataaaagtgtctcactaattctaggactgcttcaacaaatagaaaactctttaggattattcccagactctcctgttcttgaaaaattagaggataacagtttaaagctaaaaaaggctttgattatgcttatcttgtctagaaaagacatgttttccaaggctgaatagacaacttactctaacgttggagttgatttgcacaccttagttttttgctcttttaagggaggaactggaaaaacaacactttctctaaacgtgggatgcaacttggcccaatttttagggaaaaaagtgttacttgctgacctagacccgcaatccaatttatcttctggattgggggctagtgtcagaagtgaccaaaaaggcttgcacgacatagtatacacatcaaacgatttaaaatcaatcatttgcgaaacaaaaaaagatagtgtggacctaattcctgcatcattttcatccgaacagtttagagaattggatattcatagaggacctagtaacaacttaaagttatttctgaatgagtactgcgctcctttttatgacatctgcataatagacactccacctagcctaggagggttaacgaaagaagcttttgttgcaggagacaaattaattgcttgtttaactccagaacctttttctattctagggttacaaaagatacgtgaattcttaagttcggtcggaaaacctgaagaagaacacattcttggaatagctttgtctttttgggatgatcgtaactcgactaaccaaatgtatatagacattatcgagtctatttacaaaaacaagcttttttcaacaaaaattcgtcgagatatttctctcagccgttctcttcttaaagaagattctgtagctaatgtctatccaaattctagggccgcagaagatattctgaagttaacgcatgaaatagcaaatattttgcatatcgaatatgaacgagattactctcagaggacaacgtgaacaaactaaaaaaagaagcggatgtcttttttaaaaaaaatcaaactgccgcttctctagattttaagaagacgcttccctccattgaactattctcagcaactttgaattctgaggaaagtcagagtttggatcgattatttttatcagagtcccaaaactattcggatgaagaattttatcaagaagacatcctagcggtaaaactgcttactggtcagataaaatccatacagaagcaacacgtacttcttttaggagaaaaaatctataatgctagaaaaatcctgagtaaggatcacttctcctcaacaactttttcatcttggatagagttagtttttagaactaagtcttctgcttacaatgctcttgcatattacgagctttttataaacctccccaaccaaactctacaaaaagagtttcaatcgatcccctataaatccgcatatattttggccgctagaaaaggcgatttaaaaaccaaggtcgatgtgatagggaaagtatgtggaatgtcgaactcatcggcgataagggtgttggatcaatttcttccttcatctagaaacaaagacgttagagaaacgatagataagtctgattcagagaagaatcgccaattatctgatttcttaatagagatacttcgcatcatgtgttccggagtttctttgtcctcctataacgaaaatcttctacaacagctttttgaactttttaagcaaaagagctgatcctccgtcagctcatatatatatatctattatatatatatatttagggatttgatttcacgagagagatttgcaactcttggtggtagactttgcaactcttggtggtagactttgcaactcttggtggtagactttgcaactcttggtggtagacttggtcataatggacttttgttaaaaaatttattaaaatcttagagctccgattttgaatagctttggttaagaaaatgggctcgatggctttccataaaagtagattgtttttaacttttggggacgcgtcggaaatttggttatctactttatcttatctaactagaaaaaattatgcgtctgggattaactttcttgtttctttagagattctggatttatcggaaaccttgataaaggctatttctcttgaccacagcgaatctttgtttaaaatcaagtctctagatgtttttaatggaaaagttgtttcagaggcatctaaacaggctagagcggcatgctacatatctttcacaaagtttttgtatagattgaccaagggatatattaaacccgctattccattgaaagattttggaaacactacattttttaaaatccgagacaaaatcaaaacagaatcgatttctaagcaggaatggacagttttttttgaagcgctccggatagtgaattatagagactatttaatcggtaaattgattgtacaag

SEQ ID NO: 2 - DNA, 1020 bp, Mycoplasma genitalium
  misc_feature (68)..(68): n is a, c, g, or t
aggatcatttggattagtaagaagccaaaatgacaacttaaatatttcaagtgttacaaagaatgttngtgatgataatctcaagtatctcaatgctgttgagaaataccttgatggtcagcaaaactttgcaatcagaaggtatgataacaacggtagagctttatatgatattaacttagcaaaaatggaaaacccctcaacggtgcaaaggggtttaaatggcgagcctatctttgatccttttaaaggctttggtttaactggtaatgcccctactgattggaatgagatcaaaggtaaagttccagtagaagtagtccaatccccccattcccccaacctctattttgtgttactagtgcctaaggtggcattagagtaccaccaacttgataagaaagtagtcaaagagagtttggaagtggaagcaacngattcttttgatccaactaaaaggttgcaaaaagatagtccaatgaaggattcaagtaaacaaggggagaaactcagtgaagcaatgtcatcagtgggtatgagtagtggtggggctacatctcctcgcaaggccctcaagatagaggtggagaaaggcagtaatgtcaatcaaggcgaactagcaaaaaacgactttgctaaaaagccactgaaacataaagaaaatagtgggacagaggtgaagttggatgcgaatggggagtttgccaatgataaagcctgaaaaccattgttgactactgatcaaatagcaaaagagaaggggatgggggcgacggtggttagtttctatgatgcaccctacagtgaaaaccatactgcctttggacttgttgatcacatcgatcctaaaaagatggttgaaaactacccaccaagttgaaagaccccgaagtgaaaccaccatgggatctgggattacaacgcaagaaacctcttgttacaaacaacagggttctttaacccaagaagacacccagagtggtttgatgaaggacaagctaaggcagataacactagccc

SEQ ID NO: 3 - DNA, 8760 bp, Mycoplasma genitalium
cattgtgttattagggattatcttttgtctgtttgccatctatgacattgcgcaagtgatcattaccattatcaatgaaggggcacttttataatctttgttatgaaaaaaggatcaataactgaagcaattaatgccattaaacaatttgataagattgttatctttcaccatgtgcgccctgatggggattgtttaggagcacaacaaggcttgtttcacctcattaaagctaactttaaaaataaggaggtgaagtgtgttggtaataacaacaacctgtttagctttatcaacatgacatttaccaaccaaattgatgagagctttttaaaagaagcacttgccattgtggtcgatgctaattacaaaaacaggattgaattgagagaactgttagataaaaacctgtttaaagcagtgttaaggattgatcaccatcccaatgaagatgatctaaacactagctttaactttgttgaagaaagctatgtagcttgttgtgagcagatagtggagatggccacagtggcgaagtggaccataccaccagtggctgctactttactatatataggtatctatacggatagtaatagatttctatatagtaatacatcatatagaacactatacttagcagcaatactatataaagctaaagctgatataaggatagtacatgatcatttaaaccatactagtttagcagatcttaagtttaaaaagtatgtttataaccactttaaaacccaaggacaagtgatctattttatctgtactaaaaagatccaaaagagactaagaatgactgcagatcaatgtgctagagttaacttgttaagtaacatagcagattacaagatctgacttttctttattgaacaagctaataatgagatcaggatagaactgaggagtaatgggattaatgtcagagatatagccattaagtatggtgggggaggacataataatgcaagtggagcgatcattactaacaaaaaacaaattagtgatgttgttagtgattgtgtgaaaaaaattgtttataattaagtttgtatgcaccaaccaaagaaaagactggctaagaagtcttgagcctttctaaccgctgcacttacccttggggttataacaggtgtaggtggttattttctctttaaccaaaataagcaacgtagtagcgtgagcaactttgcttaccaacccaagcagttaagtgttaaacaccaacaagcagttgatgaaaccttaaccccttggacttgaaacaataacaacttctcttcactaaagattactggagagaacccaggatcatttggattagtaagaagccaaaatgacaacttaaatatttcaagtgttacaaagaattctagtgatgataatctcaagtatctcaatgctgttgagaaataccttgatggtcagcaaaactttgcaatcagaaggtatgataacaacggtagagctttatatgatattaacttagcaaaaatggaaaacccctcaacggtgcaaaggggtttaaatggcgagcctatctttgatccttttaaaggctttggtttaactggtaatgcccctactgattggaatgagatcaaaggtaaagttccagtagaagtagttcaatccccccattcccccaacctctattttgtgttactagtgcctaaggtggcattagagtatcacaacctgaataaccaagtagtcaaagagagtttggaagtgaaagcaacccaatcatccttcaaccccacccaaaggttgcaaaaagatagtccagtgaaggattcaagtaaacaaggggagaaactcagtgaaacaactgcttcatccatgagtagtggtatggctacatccactcgagccaaggccctcaaagtggaggtggaaagggggagtcaaagtgattcacttttaaaaaacgactttgctaaaaagccactaaagcataagaacagtagtggggaggtgaagttagaggcagagaaggagtttactgaggcctgaaaaccattgttgactactgatcaaatagcaagagagaaggggatgggggcgacggtggttagtttctatgatgcaccctacagtgaaaaccatactgcctttggacttgttgatcacatcgatcctaaaaagatggttgaaaactacccaccaagttgaaagaccccgaagtgaaaccaccatgggatctgggattacaacgcaagaaacctcttgttacaaacaacagggttctttaacccaagaagacacccggagtggtttgatgaaggacaagctaaggcagataacactagccctggctttaaggtaggggatactgatcacaaaaaagacgggtttaaaaaaaactcttcttctccaatagctttaccatttgaagcatactttgctaacattggtaacatggttgctattggtaactcggtatttatctttggtggtaatggtcatgctactaagatgtttaccaccaatcccttaagtattggggtatttaggattaaatacactgataactttagtaagtcatcagtaacaggttgaccatatgcagtgttatttgggggattaattaatccccaaaccaatggcttgaaagatcttccccttggtaccaacaggtggtttgaatatgtaccaagaatggcagttagtggggtgaaatgggttggtaatcaactagtgttagcaggaacactaacaatgggtgatacagctactgtacctaggttaaagtatgatcaactagaaaaacacttaaacctagttgctcaaggccagggactattgagagaagacttgcagatcttcactccctatgggtgagctaatcgtcctgatattcctgtaggagcatgactccaagatgaaatgggcagtaaatttggtccccattacttcttaaataaccctgatatccaggacaatgttaataatgatacggttgaagcattaatcagtagttacaaaaacactgataagttaaaacacgtttatccttatcgatacagtggtttgtatgcttgacagttatttaactggtctaacaaactaaccaacactcccctatcagctaactttgttaatgaaaacagttatgcaccaaacagtttgtttgctgctatcttaaatgaagatctgttaacagggctaagtgataagattttctatggtaaggagaatgagtttgctgaaaatgaagcagataggtttaaccaacttttaagtttaaatcctaatcctaacactaactgagctaggtatttaaacgtagtacaacgttttactaccggacctaaccttgatagttctaccttcgatcagttcttagactttctcccctgaatcggcaatggtaaacccttttccaactccccctccccttcaacttccgcttcctcttctacccccctccccactttttctaacatcaatgttggggttaaatcaatgatcactcaacatttaaataaagaaaacacccggtgggtgtttatacctaacttttcacctgacatctgaacaggagcagggtatcgcgttcaaagtgctaatcagaaaaacggcattccttttgaacaggtgaaacctagcaataatagtaccccctttgatcccaattcagatgataataaagtcacaccatcaggtggctcctccaaaccaaccacctatcctgctttacccaacagtatcagtcccaccagtgactggatcaatgcattgactttcactaataagaataacccgcagcgcaatcaactgttgctcagaagcttactaggaactattccggtcttgatcaataagagtggggatagtaatgatcaatttaacaaggatagtgagcagaaatgggataaaactgagacaaatgagggtaatttacctgggtttggggaggtgaatgggttgtataatgccgcattactccatacctatggtttttttggcaccaataccaactctactgatcctaagataggttttaaagctgatagtagtagtagtagtagtagtacactagtaggtagtgggttaaactgaactagtcaggatgtaggtaatcttgttgtaatcaatgacaccagctttgggtttcaacttggtggttggtttattaccttcactgactttatcagaccaagaactggttatctagggattaccttaagtagcttacaagatcaaaccattatctgagcagatcagccttgaactagtttcaaaggcagttatctagacagtgatggtacccctaaatcactgtgagatccaactgctttaaaatcccttccaaatagttcaactacctatgataccaatcctaccctctcaccctccttccaactctaccaacccaacaaggtgaaggcttaccaaaccactaacacctacaacaagttaattgaaccagttgatgcaacaagtgcagcaactaacatgaccagtttgttaaaactcctaacaactaaaaacatcaaagcgaaattggggaagggaacagcttcttcgcagggaaataataatggagggggtgttagtcaaacgattaacaccatcaccactacgggaaatattagtgaaggtctaaaagaagaaactagtattcaagcagaaacacttaaaaagttctttgatagtaaacaaaacaataagagtgaaatagggataggtgatagtacatttaccaagatggatggtaaactaactggcgtagtatctactccccttgttaaccttatcaatggccagggagcaactagtgatagtgatactgaaaaaattagctttaaacctggtaaccagattgactttaataggttattcaccttaccagtaactgaactatttgatcctaacacgatgtttgtctatgaccagtatgtaccactattggttaacttacctagtggctttgatcaagcttcaatccgcttaaaggtaattagttactcagtagaaaaccaaaccttaggagttagattagagttcaaagatcctcaaacccaacagtttatcccggtactaaatgcatcaagtacaggtccccaaactgtctttcaaccctttaaccagtgggcagactatgtcttacctttgattgtaactgttcctatagtagtgattatccttagtgttactttgggattaacgattggaattccaatgcacagaaacaaaaaggcattacaagcagggtttgatctttctaacaaaaaggttgatgtcttgaccaaagcagttggtagtgtctttaaagagatcattaacagaacagggatctctaacgctcctaagaagttaaaacaagctaccccaaccaaaccaactcctaaaaccccaccaaaacctccagtaaaacaataagatgaaaacaatgagaaaacagatttataaaaaagcatactggttactattaccctttctaccattagcactagccaataccttccttgtcaaagaggatagtaagaatgttactgcttacacccccttcgccacccccatcaccgattctaaaagtgatctggttagtttggcacaacttgattcttcttatcaaatcgctgaccaaaccatccataacaccaacctgtttgtgttgttcaagtctagggatgtaaaagttaagtatgagtcaagtggcagtaacaacattagttttgattcaactagtcaaggtgaaaaaccctcctatgtggtcgagtttactaactctaccaacattggcatcaagtgaacgatggtgaaaaagtatcagttagatgtaccgaatgtaagtagtgacatgaaccaagtactgaaaaatttaattcttgaacaacctttgactaagtataccttaaacagtagtttggccaaagagaagggcaaaacgcaaagggaggtacatctgggtagtgggcaagcaaatcagtgaaccagtcaacgcaaccaacatgacctaaacaacaatcccagtcccaatgcttcaactgggtttaaactcactaccggcaatgcatatagaaaactaagtgagtcctgaccaatttatgaaccaattgatgggaccaagcagggcaaagggaaggatagtagtgggtggagttcaactgaagaaaacgaagctaaaaatgatgcgcccagtgtttctggagggggatcatcttctggaacatttaataaatacctcaacaccaagcaagcgttagagagcatcggtatcttgtttgatgatcaaaccccaagaaatgttatcacccaactctattatgcttctactagcaagctagcagtcaccaacaaccacattgtcgtgatgggtaacagctttctacccagcatgtggtactgggtggtggagcggagtgcacaggaaaatgcaagtaacaaacccacctggtttgctaataccaatttagactgaggagaagacaaacaaaaacaatttgttgagaaccagttggggtataaggaaactaccagtaccaattcccacaacttccattccaaatctttcacccaacctgcatatctgatcagtggcattgacagtgtcaatgatcaaatcatcttcagtggctttaaagcggggagtgtggggtatgatagtagtagtagtagtagtagtagtagtagtagtaccaaagaccaagcacttgcttgatcaacaacaactagcttagatagtaaaacggggtataaggatctagtgaccaacgacacggggctaaatggtccgatcaatgggagtttttcaatccaagacaccttcagctttgttgttccttattcggggaatcatacaaataatggaacaactggacccattaaaactgcttatccagtgaaaaaagatcaaaaatcaactgtcaagatcaattctttgattaacgctacgcccttgaatagttatggggatgaggggattggggtgtttgatgcgttaggtttaaactataactttaaatctaaccaagaacgtttaccttccagaactgatcagatctttgtttatgggattgtctcccctaatgaattgcgaagtgctaaaagttctgctgattcaactggtagtgatacaaaggtaaactgatcaaacacccaatcacgttacctccctgttccctataactattcagaagggatcattgatgcagatggatttaagcgtcctgaaaacaggggtgctagtgtaactaccttctcagggcttaaatcaattgcccctgatggttttgctaactcaatagctaacttctcagttgggttaaaagcaggaattgatcctaacccagtgatgagtggtaagaaagctaactatggagcggttgtgttaacacgggggggtgttgttagattaaactttaaccctggtaatgattcattgctttcaacaactgataacaatatagcacctatctccttctcatttactccgttcacagctgctgagagtgcggtggatctcactaccttcaaagaagttacctataaccaagaatcagggttatggagttatatctttgacagctccttaaaaccaagccatgatggtaaacaaactcctgtcactgataacatgggctttagtgttatcactgtctcaagaactggcattgaactaaaccaagaccaagctactacaactcttgatgtagcacctagtgcactagcagtgcaatcagggatccaatctaccacccaaaccctaactggagtactcccacttagtgaggaattcagtgcagttattgctaaagatagtgatcaaaataagattgatatctataaaaacaacaacgggttgtttgaaattgatacccaactaagtaatagtgttgccaccaacaacggtgggttagcacctagttacacagaaaacagggttgatgcatggggtaaagttgagtttgctgataacagtgtattgcaagcaagaaacctagttgataaaactgttgatgagatcatcaatacccctgaaatcttaaactccttctttagattcacccctgcttttgaagatcaaaaagctacccttgttgctactaagcaaagtgatacatcacttagtgtctcaccaaggatccagttcttagatggtaatttctatgatcttaactctaccatcgctggggtacctttaaacattggtttcccttcaagagtgtttgctgggtttgcagcactccctgcatgggtgatccctgtatcagtaggttcttcagttgggatcttgtttatcttgttagtcttaggacttgggattgggatcccaatgtacagggtaagaaaactccaagatgcatcgtttgttaatgtctttaaaaaggttgatacactcacaactgctgtcggtagtgtgtacaaaaagattattacccaaactggtgtggtgaaaaaagcacctagtgcattgaaagctgctaatcctagtgttaaaaaacctgctgcttttttaaaaccacctgttcaaccaccaagtaaacctgaaggggaacaaaaagctgttgaagttaagtcagaagaaaccaaaagttagtttttaacctttcaataacctaaaacacaatctttaaaacaaggttgtgtttttttgttttttgtcacttttcactaaacttgcaatttagagagtggatatgaaaagaacagttaaaaaaataaaacctgaccacttgtttaataaaaagcagtggcacttactgagtgaagagatcagtgataacccaatgattaagc

SEQ ID NO: 4 - DNA, 453 bp, Neisseria gonorrhoeae
gtgatcgctatcgtcggcattttggcggcagtcgcccttcccgcctaccaagactacaccgcccgcgcgcaagtttccgaagccatccttttggccgaaggtcaaaaatcagccgtcaccgagtattacctgaatcacggcaaatggccggaaaacaacacttctgccggcgtggcatcctcccccaccgacatcaaaggcaaatatgttaaagaggttgaagttaaaaacggcgtcgttaccgccacaatggcctcaagcaacgtaaacaatgaaatcaaaggcaaaaaactctccctgtgggccaggcgtgaaaacggttcggtaaaatggttctgcggacagccggttacgcgcaccgacgacgacaccgttgccgacgccaaagacggcaaagaaatcgacaccaagcacctgccgtcaacctgccgcgacaacaaagatgccaaatga

SEQ ID NO: 5 - DNA, 190 bp, Chlamydia trachomatis
cttaaagttatttctgaatgagtactgcgctcctttttatgacatctgcataatagacactccacctagcctaggagggttaacgaaagaagcttttgttgcaggagacaaattaattgcttgtttaactccagaacctttttctattctagggttacaaaagatacgtgaattcttaagttcggtcgga

SEQ ID NO: 6 - DNA, 175 bp, Chlamydia trachomatis
atttctgaatgagtactgcgctcctttttatgacatctgcataatagacactccacctagcctaggagggttaacgaaagaagcttttgttgcaggagacaaattaattgcttgtttaactccagaacctttttctattctagggttacaaaagatacgtgaattcttaagttcg

SEQ ID NO: 7 - DNA, 20 bp, Chlamydia trachomatis
atttctgaatgagtactgcg

SEQ ID NO: 8 - DNA, 26 bp, Chlamydia trachomatis
catctgcataatagacactccaccta

SEQ ID NO: 9 - DNA, 36 bp, Chlamydia trachomatis
cgcgccatctgcataatagacactccacctagcgcg

SEQ ID NO: 10 - DNA, 27 bp, Chlamydia trachomatis
cctagcctaggagggttaacgaaagaa

SEQ ID NO: 11 - DNA, 39 bp, Chlamydia trachomatis
acgcgccctagcctaggagggttaacgaaagaagcgcgt

SEQ ID NO: 12 - DNA, 22 bp, Chlamydia trachomatis
cgaacttaagaattcacgtatc

SEQ ID NO: 13 - DNA, 270 bp, Mycoplasma genitalium
  misc_feature (68)..(68): n is a, c, g, or t
aggatcatttggattagtaagaagccaaaatgacaacttaaatatttcaagtgttacaaagaatgttngtgatgataatctcaagtatctcaatgctgttgagaaataccttgatggtcagcaaaactttgcaatcagaaggtatgataacaacggtagagctttatatgatattaacttagcaaaaatggaaaacccctcaacggtgcaaaggggtttaaatggcgagcctatctttgatccttttaaaggctttggtttaactggtaa

SEQ ID NO: 14 - DNA, 258 bp, Mycoplasma genitalium
  misc_feature (67)..(67): n is a, c, g, or t
ggatcatttggattagtaagaagccaaaatgacaacttaaatatttcaagtgttacaaagaatgttngtgatgataatctcaagtatctcaatgctgttgagaaataccttgatggtcagcaaaactttgcaatcagaaggtatgataacaacggtagagctttatatgatattaacttagcaaaaatggaaaacccctcaacggtgcaaaggggtttaaatggcgagcctatctttgatccttttaaaggctttggt

SEQ ID NO: 15 - DNA, 24 bp, Mycoplasma genitalium
ggatcatttggattagtaagaagc

SEQ ID NO: 16 - DNA, 27 bp, Mycoplasma genitalium
cagaaggtatgataacaacggtagagc

SEQ ID NO: 17 - DNA, 39 bp, Mycoplasma genitalium
tgcgcacagaaggtatgataacaacggtagagctgcgca

SEQ ID NO: 18 - DNA, 23 bp, Mycoplasma genitalium
accaaagcctttaaaaggatcaa

SEQ ID NO: 19 - DNA, 151 bp, Mycoplasma genitalium
aacaggtgtaggtggttattttctctttaaccaaaataagcaacgtagtagcgtgagcaactttgcttaccaacccaagcagttaagtgttaaacaccaacaagcagttgatgaaaccttaaccccttggacttgaaacaataacaacttc

SEQ ID NO: 20 - DNA, 140 bp, Mycoplasma genitalium
ggtgtaggtggttattttctctttaaccaaaataagcaacgtagtagcgtgagcaactttgcttaccaacccaagcagttaagtgttaaacaccaacaagcagttgatgaaaccttaaccccttggacttgaaacaataa

SEQ ID NO: 21 - DNA, 21 bp, Mycoplasma genitalium
ggtgtaggtggttattttctc

SEQ ID NO: 22 - DNA, 25 bp, Mycoplasma genitalium
accaacccaagcagttaagtgttaa

SEQ ID NO: 23 - DNA, 37 bp, Mycoplasma genitalium
cgcgttaccaacccaagcagttaagtgttaaaacgcg

SEQ ID NO: 24 - DNA, 22 bp, Mycoplasma genitalium
ttattgtttcaagtccaagggg

SEQ ID NO: 25 - DNA, 191 bp, Mycoplasma genitalium
gtttgtatgcaccaaccaaagaaaagactggctaagaagtcttgagcctttctaaccgctgcacttacccttggggttataacaggtgtaggtggttattttctctttaaccaaaataagcaacgtagtagcgtgagcaactttgcttaccaacccaagcagttaagtgttaaacaccaacaagcagttga

SEQ ID NO: 26 - DNA, 186 bp, Mycoplasma genitalium
gtatgcaccaaccaaagaaaagactggctaagaagtcttgagcctttctaaccgctgcacttacccttggggttataacaggtgtaggtggttattttctctttaaccaaaataagcaacgtagtagcgtgagcaactttgcttaccaacccaagcagttaagtgttaaacaccaacaagcagttg

SEQ ID NO: 27 - DNA, 24 bp, Mycoplasma genitalium
gtatgcaccaaccaaagaaaagac

SEQ ID NO: 28 - DNA, 27 bp, Mycoplasma genitalium
caggtgtaggtggttattttctcttta

SEQ ID NO: 29 - DNA, 39 bp, Mycoplasma genitalium
cgcgttcaggtgtaggtggttattttctctttaaacgcg

SEQ ID NO: 30 - DNA, 24 bp, Mycoplasma genitalium
caactgcttgttggtgtttaacac

SEQ ID NO: 31 - DNA, 191 bp, Mycoplasma genitalium
caacggtgcaaaggggtttaaatggcgagcctatctttgatccttttaaaggctttggtttaactggtaatgcccctactgattggaatgagatcaaaggtaaagttccagtagaagtagttcaatccccccattcccccaacctctattttgtgttactagtgcctaaggtggcattagagtatcacaac

SEQ ID NO: 32 - DNA, 178 bp, Mycoplasma genitalium
gcaaaggggtttaaatggcgagcctatctttgatccttttaaaggctttggtttaactggtaatgcccctactgattggaatgagatcaaaggtaaagttccagtagaagtagttcaatccccccattcccccaacctctattttgtgttactagtgcctaaggtggcattagagtat

SEQ ID NO: 33 - DNA, 24 bp, Mycoplasma genitalium
gcaaaggggtttaaatggcgagcc

SEQ ID NO: 34 - DNA, 26 bp, Mycoplasma genitalium
atgagatcaaaggtaaagttccagta

SEQ ID NO: 35 - DNA, 38 bp, Mycoplasma genitalium
agcgtcatgagatcaaaggtaaagttccagtagacgct

SEQ ID NO: 36 - DNA, 24 bp, Mycoplasma genitalium
atactccaataccaccttaggcac

SEQ ID NO: 37 - DNA, 211 bp, Mycoplasma genitalium
agcaaaaatggaaaacccctcaacggtgcaaaggggtttaaatggcgagcctatctttgatccttttaaaggctttggtttaactggtaatgcccctactgattggaatgagatcaaaggtaaagttccagtagaagtagttcaatccccccattcccccaacctctattttgtgttactagtgcctaaggtggcattagagtatcacaac

SEQ ID NO: 38 - DNA, 204 bp, Mycoplasma genitalium
gcaaaaatggaaaacccctcaacggtgcaaaggggtttaaatggcgagcctatctttgatccttttaaaggctttggtttaactggtaatgcccctactgattggaatgagatcaaaggtaaagttccagtagaagtagttcaatccccccattcccccaacctctattttgtgttactagtgcctaaggtggcattagagtat

SEQ ID NO: 39 - DNA, 25 bp, Mycoplasma genitalium
gcaaaaatggaaaacccctcaacgg

SEQ ID NO: 40 - DNA, 27 bp, Mycoplasma genitalium
gattggaatgagatcaaaggtaaagtt

SEQ ID NO: 41 - DNA, 39 bp, Mycoplasma genitalium
cgccctgattggaatgagatcaaaggtaaagttagggcg

SEQ ID NO: 42 - DNA, 280 bp, Neisseria gonorrhoeae
gtcaaaaatcagccgtcaccgagtattacctgaatcacggcaaatggccggaaaacaacacttctgccggcgtggcatcctcccccaccgacatcaaaggcaaatatgttaaagaggttgaagttaaaaacggcgtcgttaccgccacaatggcctcaagcaacgtaaacaatgaaatcaaaggcaaaaaactctccctgtgggccaggcgtgaaaacggttcggtaaaatggttctgcggacagccggttacgcgcaccgacgacgacaccgttgccga

SEQ ID NO: 43 - DNA, 252 bp, Neisseria gonorrhoeae
cgtcaccgagtattacctgaatcacggcaaatggccggaaaacaacacttctgccggcgtggcatcctcccccaccgacatcaaaggcaaatatgttaaagaggttgaagttaaaaacggcgtcgttaccgccacaatggcctcaagcaacgtaaacaatgaaatcaaaggcaaaaaactctccctgtgggccaggcgtgaaaacggttcggtaaaatggttctgcggacagccggttacgcgcaccgacga

SEQ ID NO: 44 - DNA, 19 bp, Neisseria gonorrhoeae
cgtcaccgagtattacctg

SEQ ID NO: 45 - DNA, 22 bp, Neisseria gonorrhoeae
ggcccacagggagagttttttg

SEQ ID NO: 46 - DNA, 34 bp, Neisseria gonorrhoeae
actgcgggcccacagggagagttttttgcgcagt

SEQ ID NO: 47 - DNA, 17 bp, Neisseria gonorrhoeae
tcgtcggtgcgcgtaac

SEQ ID NO: 48 - DNA, 92 bp, Artificial
  IC sequence (92nt)
attggatcccagcacgctaattacccagtatcagatgacgtggcagccatgagagtgggacagtcgtccttcaaggagcacatcagcggatc

SEQ ID NO: 49 - DNA, 17 bp, Artificial
  IC forward primer IS 368
cagcacgctaattaccc

SEQ ID NO: 50 - DNA, 23 bp, Artificial
  IC probe
cccactctcatggctgccacgtc

SEQ ID NO: 51 - DNA, 35 bp, Artificial
  IC probe in beacon format
caggcgcccactctcatggctgccacgtccgcctg

SEQ ID NO: 52 - DNA, 17 bp, Artificial
  IC reverse primer IS 569
gctgatgtgctccttga

SEQ ID NO: 53 - DNA, 18 bp, Artificial
  16S rRNA forward primer S1-F
agtttgatcatggctcag

SEQ ID NO: 54 - DNA, 17 bp, Artificial
  16S rRNA reverse primer S1-R
gtattaccgcggctgct

SEQ ID NO: 55 - DNA, 25 bp, Mycoplasma genitalium
agttgatgaaaccttaaccccttgg

SEQ ID NO: 56 - DNA, 25 bp, Mycoplasma genitalium
ccgttgaggggttttccatttttgc

SEQ ID NO: 57 - DNA, 25 bp, Mycoplasma genitalium
gaccatcaaggtatttctcaacagc

(19) United States
(12) Patent Application Publication
(10) Pub. No.: US 2014/0309136 A1
(43) Pub. Date: Oct. 16, 2014

(54) REAL-TIME MULTIPLEX DETECTION OF THREE BACTERIAL SPECIES RESPONSIBLE FOR SEXUALLY TRANSMITTED DISEASES

(71) Applicant: BIO-RAD INNOVATIONS, Marnes-La-CoQuette (FR)

(72) Inventor: Gervais CLAREBOUT, Blainville Sur Orne (FR)

(73) Assignee: BIO-RAD INNOVATIONS, Marnes-La-CoQuette (FR)

(21) Appl. No.: 14/284,720

(22) Filed: May 22, 2014

Related U.S. Application Data
(62) Division of application No. 12/227,310, filed on Nov. 13, 2008, now abandoned.

(30) Foreign Application Priority Data
May 29, 2006 (EP) 06290862.9

(51) Int. Cl. C12Q 1/68

(52) U.S. Cl. CPC C12Q 1/689
USPC 506/9; 506/23

(57) ABSTRACT

The invention relates to the detection of three different bacterial species which are responsible for sexually-transmitted diseases, i.e., Chlamydia trachomatis (CT), Neisseria gonorrhoeae (NG) and Mycoplasma genitalium (MG). The invention more particularly relates to the detection of these three species in real-time PCR, in multiplex PCR and in real-time multiplex PCR. The invention provides reference templates sequences, which are especially adapted to the design of primers and probes, which can be used together in the same tube to detect CT and/or MG and/or NG by real-time multiplex amplification.

This application is a divisional of application Ser. No. 12/227,310 (pending), filed Nov. 13, 2008 (published as US 2009-0104610 A1), which is a U.S. national phase of International Application No. PCT/EP2007/003170, filed 10 Apr. 2007, which designated the U.S. and claims priority to Europe Application No. 06290862.9, filed 29 May 2006, the entire contents of each of which are hereby incorporated by reference.

### TECHNICAL FIELD

The present invention relates to the detection of three different bacterial species which are responsible for sexually-transmitted diseases, i.e., Chlamydia trachomatis (CT), Neisseria gonorrhoeae (NG) and Mycoplasma genitalium (MG).

The present invention more particularly relates to the detection of these three species in real-time PCR, in multiplex PCR or in real-time multiplex PCR.

### BACKGROUND OF THE INVENTION

Chlamydia trachomatis (CT) is a species of the chlamydiae, a group of obligately intracellular bacteria. It causes sexually transmitted diseases, such as chlamydia and lymphogranuloma venereum, as well as trachoma, an eye infection that is a frequent cause of blindness.

Neisseria gonorrhoeae (NG) is a species of Gram-negative bacteria responsible for the disease gonorrhoea.

CT and NG co-infections are frequent.

Mycoplasma genitalium (MG) is a parasitic bacterium which lives in the primate genital and respiratory tracts. MG is thought to be involved in urethritis.

Traditional diagnosis of the presence of CT and/or MG and/or NG involves the culture of samples collected from patients, such as urethral specimen, on species-specific culture media. Such cultures are time-consuming and fastidious. They are all the most time-consuming and fastidious in the case of CT, MG and NG, because the culture of CT and MG requires a high level of technicality, and because NG is very sensitive to temperature and humidity variations.

Diagnosis methods based on nucleic acid amplification have therefore been developed.

For example, the FDA-approved Amplicor™ kit available from Roche Diagnostic enables the detection of CT or NG. However, it does not enable to detect MG.

WO 98/11259 in the name of Visible Genetics Inc. discloses a method for co-amplification and detection of CT, MG and NG. This method involves the amplification of CT, MG and NG targets by amplification primers. The detection of the amplicons produced by said primers is carried out either by direct labelling of the primers, or by agarose gel techniques. The method of WO 98/11259 does however not involve the use of amplicon-annealing probes (cf. pages 9-10 of WO 98/11259). Hence, the method of WO 98/11259 is not a real-time technique.

The present invention enables the detection of CT, MG and NG in real-time amplification, and provides primers as well as probes, preferably beacon probes, which can be used together in multiplex in the same tube to detect the three bacterial species in real-time amplification.

As an advantageous feature, the invention may thus be implemented in real-time PCR, in multiplex PCR, or in multiplex real-time PCR.

### SUMMARY OF THE INVENTION

The invention relates to the detection of three different bacterial species which are responsible for sexually-transmitted diseases, i.e., Chlamydia trachomatis (CT), Neisseria gonorrhoeae (NG) and Mycoplasma genitalium (MG).

The invention more particularly relates to the detection of these three species in real-time PCR, in multiplex PCR and in real-time multiplex PCR.

The invention provides reference templates sequences, which are especially adapted to the design of primers and probes, which can be used together in the same tube to detect CT and/or MG and/or NG, advantageously CT and MG and NG, by real-time multiplex amplification.

The invention also relates to these primers and probes, as well as to pharmaceutical compositions, to biological compositions, to detection kits and to diagnostic kits, which comprise at least one of primers and/or probes of the invention.

Table 1, which is at the end of Example 1 below, lists SEQ ID sequences, which are representative of reference template polynucleotides, primers and probes of the invention.

The invention further relates to a process for the detection of CT, MG and NG, which involves the use of at least one primer pair and/or at least one probe of the invention, as well as to the amplicons, which are obtainable with the primers of the invention.

To the best of the inventors' knowledge, the invention provides the first description of a detection of CT and MG and NG in real-time multiplex amplification.

### BRIEF DESCRIPTION OF THE FIGURES

FIG. 1 shows the sequence, which is available under accession number J03321 (SEQ ID NO: 1), which is the pCHL1 plasmid sequence of CT.

FIG. 2 shows the sequence, which is available under accession number X91074 (SEQ ID NO: 2), which is the sequence of the 5′ region of the adhesin gene of MG.

FIG. 3 shows the sequence, which is available under accession number M31431 (SEQ ID NO: 3), which is the sequence of the adhesin gene of MG.

FIG. 4 shows the sequence, which is available under accession number AF042097 (SEQ ID NO: 4), which is the sequence of the pilE gene of NG.

### DETAILED DESCRIPTION

The present invention relates to the detection of three sexually-transmittable bacterial species, namely: Chlamydia trachomatis (CT), Mycoplasma genitalium (MG) and Neisseria gonorrhoeae (NG).

The present invention provides nucleic acid reference template sequences, which allow for the construction and production of primers and probes, which are suitable for the detection of CT and/or MG and/or NG in real-time multiplex amplification.

The present invention thus provides CT primers, CT primer pairs, CT probes, MG primers, MG primer pairs, MG probes, NG primers, NG primer pairs and NG probes, which are suitable for the detection of CT and/or MG and/or NG in real-time multiplex amplification.

The primers of the invention can be mixed together in multiplex in the same tube to amplify CT and MG and NG in said same tube.

Advantageously, the present invention provides probes, which are suitable for real-time detection in such multiplex operative conditions.

Hence, the primers and probes of the invention can be mixed together in multiplex in the same tube to amplify and detect CT and MG and NG in real-time in said same tube.

Hence, the present invention enables the detection of CT and MG and NG in one single (amplification+detection) operative step.

To the best of the inventors' knowledge, the present invention is the first description of a real-time multiplex detection of the three bacterial species (CT, MG, NG).

In addition to detecting the three bacterial species within the same sample, the invention has the advantage of allowing checking for the absence of Taq polymerase inhibitors, by the use of an Internal Control (IC). IC, as well as IC primers and probes, are described in more details below.

The invention therefore allows for a quadruplex amplification (CT, MG, NG and IC).

The invention provides for a detection of said three bacterial species, which is much faster than any prior art technique, as well as much reliable as it heavily reduces the risk of having contaminated samples or cultures for analysis.

The means of the invention further are very sensitive and reproducible (see example 4 below).

At present time, there is no systemic detection of MG, whereas it is now acknowledged that MG is responsible for non-gonococcal urethritis, and is often associated to cervicitis and endometritis.

The present invention provides the first means that allow for a systemic detection of MG in a routine test allowing the simultaneous detection of CT and NG.

The invention thereby allows the physician to avoid the prescription of inappropriate antibiotics.

More particularly, the present invention provides:
CT real-time amplification systems, wherein each CT real-time amplification system comprises at least two CT primers of the invention and at least one CT probe of the invention,
MG real-time amplification systems, wherein each MG real-time amplification system comprises at least two MG primers of the invention and at least one MG probe of the invention, and
NG real-time amplification systems, wherein each NG real-time amplification system comprises at least two NG primers and at least one NG probe.

Still more particularly, the present invention provides CT real-time amplification systems, MG real-time amplification systems, and NG real-time amplification systems, which allow for a detection of CT, MG and NG, respectively, which is specific of the bacterial species to which the real-time amplification system is intended.

Advantageously, the present invention provides CT real-time amplification systems, MG real-time amplification systems, and NG real-time amplification systems, which can be used together in multiplex in the same tube, without any significant loss in specificity: even when used together in multiplex in the same tube,
such a CT real-time amplification system of the invention still specifically detects CT, without any significant cross-reactivity with the real-time amplification systems of MG and NG, or with the amplicons that may possibly be produced by these MG and NG systems,
such a MG real-time amplification system of the invention still specifically detects MG, without any significant cross-reactivity with the real-time amplification systems of CT and NG, or with the amplicons that may possibly be produced by these CT and NG systems,
such a NG real-time amplification system of the invention still specifically detects NG, without any significant cross-reactivity with the real-time amplification systems of CT and MG, or with the amplicons that may possibly be produced by these CT and MG systems.

To the best of the inventors' knowledge, none of the real-time amplification systems of the invention detects human DNA (no cross-hybridization).

The application also relates to pharmaceutical compositions, to biological compositions, to detection kits and to diagnostic kits, which comprise at least one of the primers and/or probes of the invention, preferably at least two primers and at least one probe of the invention, more preferably at least one real-time amplification system of the invention, most preferably at least one CT and at least one MG and at least one NG real-time amplification systems of the invention.

The present application also relates to a process for the detection of at least one Chlamydia trachomatis (CT) and/or at least one Mycoplasma genitalium (MG) and/or at least one Neisseria gonorrhoeae (NG).

Said detection is usually performed in a sample.

By “sample containing nucleic acid material”, it is meant any sample, which contains at least one nucleic acid, e.g., a biological sample, such as a sample which has been collected from a cell culture, or from an animal or a human being, preferably a sample which has been collected from a tissue or fluid that is suspected of containing CT and/or MG and/or NG, such as e.g., a sample of uterine cervix, most preferably a urine sample (such as a first void urine sample).

Advantageously, the invention enables a reliable detection of CT and/or MG and/or NG is a urine sample.

Said sample may optionally have been further treated and/or purified according to any technique known by the skilled person, to improve the amplification efficiency and/or qualitative accuracy and/or quantitative accuracy. The sample may thus exclusively, or essentially, consist of nucleic acid(s), whether obtained by purification, isolation, or by chemical synthesis. Means are available to the skilled person, who would like to isolate or purify nucleic acids, such as DNA, from a biological sample, for example to isolate or purify DNA from cervical scrapes (e.g., QIAamp-DNA Mini-Kit; Qiagen, Hilden, Germany).

Said detection comprises the determination of whether at least one amplicon has been, or is, produced from said sample, or from nucleic acid material thereof, by amplification by means of amplification primers,

whereby a positive determination indicates that at least one CT and/or at least one MG and/or at least one NG is(are) present in said sample.

Said determination can be carried out by means of at least one probe, which is intended to anneal to said at least one amplicon.

The detection process of the invention may thus comprise:
contacting said sample, or nucleic acid material thereof, with at least two amplification primers, under conditions suitable for the production of at least one amplicon by said primers (i.e., under conditions, which would be suitable for the production by said primers of at least one amplicon from said at least one CT and/or MG and/or NG to be detected, if this CT and/or MG and/or NG were present in said sample),
determining whether at least one amplicon has been produced, or is produced, by said primers, e.g., by means of at least one detection probe, which anneals to the amplicon produced by said at least two primers, preferably in real-time amplification involving at least one probe of the invention.

In the process of the invention, said amplification primers comprise:
at least two primers, which are intended for targeting CT, and which are suitable for the detection of CT in real-time multiplex amplification, and/or
at least two primers, which are intended for targeting MG, and which are suitable for the detection of MG in real-time multiplex amplification, and/or
at least two primers, which are intended for targeting NG, and which are suitable for the detection of NG in real-time multiplex amplification.

In the process of the present invention, said at least one probe preferably comprise:
at least one CT-targeted probe, which is suitable for the detection of CT in real-time multiplex amplification, and/or
at least one MG-targeted probe, which is suitable for the detection of MG in real-time multiplex amplification, and/or
at least one NG-targeted probe, which is suitable for the detection of NG in real-time multiplex amplification.

Whereas the reference template sequences, the primers and the probes of the invention have been optimized so as to allow for the detection of CT, MG and NG in real-time multiplex amplification, the simplex embodiments thereof are of course also encompassed by the application (e.g., real-time, or non real-time simplex embodiments, involving one primer pair of the invention).

Quite similarly, whereas the present invention provides the special feature of allowing a multiplex detection of CT, MG and NG in real-time, the implementations of the primers of the invention without a probe of the invention, or with at least one probe of the invention but not in real-time, are of course also encompassed by the present application.

Also, whereas the primers of the invention have been designed as primer pairs, each primer is individually encompassed as such by the present application.

Table 1, which is at the end of Example 1 below, lists SEQ ID sequences, which are representative of reference template polynucleotides, primers and probes of the invention. Table 1 thereby shows two CT real-time amplification systems, five MG real-time amplification systems, and one real-time amplification system of the invention. These systems can be used together in the same tube to detect CT and MG and NG in real-time multiplex amplification.

Said at least two primers, which are intended for targeting CT, and which are suitable for the detection of CT in real-time multiplex amplification, are oligonucleotides, the sequences of which are suitable for use as forward and reverse primers, respectively, in the amplification of at least one CT reference template sequence, wherein said at least one CT reference template sequence is a fragment consisting of positions 5571-5760 (SEQ ID NO: 5) of the CT sequence of SEQ ID NO: 1 (CT pCHL1 plasmid; J03321), or of a conservative sub-fragment thereof, which has retained the property of being a suitable reference template sequence, to construct and produce CT-targeted primers, which allow for a real-time multiplex detection of CT. Said at least two primers, which are intended for targeting CT, preferably consist of 14-30 nucleotides (each independently from each other).

Said at least one CT reference template sequence advantageously is the fragment consisting of positions 5580-5754 (SEQ ID NO: 6) of the CT sequence of SEQ ID NO: 1, or the sequence that is fully complementary to said fragment over the entire length of said fragment.

Said at least two primers, which are intended for targeting MG, and which are suitable for the detection of MG in real-time multiplex amplification, are oligonucleotides, the sequences of which are suitable for use as forward and reverse primers, respectively, in the amplification of at least one MG reference template sequence, wherein said at least one MG reference template sequence is a fragment consisting of:
positions 1-270 (SEQ ID NO: 13) of the MG sequence of SEQ ID NO: 2 (5′ region of MG adhesin gene; X91074), or of a conservative sub-fragment thereof, which has retained the property of being a suitable reference template sequence, to construct and produce MG-targeted primers, which allow for a real-time multiplex detection of MG,
positions 1140-1290 (SEQ ID NO: 19) of the MG sequence of SEQ ID NO: 3 (MG adhesion gene; M31431), or of a conservative sub-fragment thereof, which has retained the property of being a suitable reference template sequence, to construct and produce MG-targeted primers, which allow for a real-time multiplex detection of MG,
positions 1060-1250 (SEQ ID NO: 25) of the MG sequence of SEQ ID NO: 3, or of a conservative sub-fragment thereof, which has retained the property of being a suitable reference template sequence, to construct and produce MG-targeted primers, which allow for a real-time multiplex detection of MG,
positions 1520-1710 (SEQ ID NO: 31) of the MG sequence of SEQ ID NO: 3, or of a conservative sub-fragment thereof, which has retained the property of being a suitable reference template sequence, to construct and produce MG-targeted primers, which allow for a real-time multiplex detection of MG,
positions 1500-1710 (SEQ ID NO: 37) of the MG sequence of SEQ ID NO: 3, or of a conservative sub-fragment thereof, which has retained the property of being a suitable reference template sequence, to construct and produce MG-targeted primers, which allow for a real-time multiplex detection of MG.

Said at least two primers, which are intended for targeting MG, preferably consist of 14-30 nucleotides (each independently from each other).

Said at least one MG reference template sequence advantageously is:
the fragment consisting of positions 2-259 (SEQ ID NO: 14) of the MG sequence of SEQ ID NO:2, or the sequence that is fully complementary to said fragment over the entire length of said fragment, or
the fragment consisting of positions 1144-1283 (SEQ ID NO:20) of the MG sequence of SEQ ID NO:3, or the sequence that is fully complementary to said fragment over the entire length of said fragment, or
the fragment consisting of positions 1064-1249 (SEQ ID NO:26) of the MG sequence of SEQ ID NO:3, or the sequence that is fully complementary to said fragment over the entire length of said fragment, or
the fragment consisting of positions 1527-1704 (SEQ ID NO:32) of the MG sequence of SEQ ID NO:3, or the sequence that is fully complementary to said fragment over the entire length of said fragment, or
the fragment consisting of positions 1501-1704 (SEQ ID NO:38) of the MG sequence of SEQ ID NO:3, or the sequence that is fully complementary to said fragment over the entire length of said fragment.

Said MG reference template sequences share the specific technical feature of being suitable references to construct and produce MG-targeted primers, as well as MG-specific probes, which can be used together in multiplex in the same tube to specifically detect CT and/or MG and/or NG, advantageously CT and MG and NG, in real-time time in said same tube.

Said at least two primers, which are intended for targeting NG, and which are suitable for the detection of NG in real-time multiplex amplification, are oligonucleotides, the sequences of which are suitable for use as forward and reverse primers, respectively, in the amplification of at least one NG reference template sequence, wherein said at least one NG reference template sequence is a fragment consisting of positions 101-380 (SEQ ID NO: 42) of the NG sequence of SEQ ID NO: 4 (NG pilE gene; AF042097), or of a conservative sub-fragment thereof, which has retained the property of being a suitable reference template sequence, to construct and produce NG-targeted primers, which allow for a real-time multiplex detection of NG.

Said at least two primers, which are intended for targeting NG, preferably consist of 14-30 nucleotides (each independently from each other).

Said at least one NG reference template sequence advantageously is the fragment consisting of positions 114-365 (SEQ ID NO: 43) of the NG sequence of SEQ ID NO: 4, or the sequence that is fully complementary to said fragment over the entire length of said fragment.

By “consisting of 14-30 nucleotides”, it is meant “consisting of 14, 15, 16, 17, 18, 19, 20, 21, 22, 23, 24, 25, 26, 27, 28, 29 or 30 nucleotides”.

The same applies mutatis mutandis to any range, which is recited in the present application.

The nucleotide lengths of the primers can be chosen independently from each other.

By “suitable for use as forward and reverse primers, respectively, in the amplification of” a reference template sequence “consisting of” an indicated sequence or SEQ ID, it is herein meant that these forward and reverse primers do not flank the reference template sequence, but that they anneal in the exact terminal positions that allow for the sequence that would be amplified to consist of the indicated reference template sequence.

More preferably, a primer of the invention consists of 14-30 nucleotides, the sequence of which has an identity of at least 80%, preferably of at least 85%, more preferably of at least 90%, even more preferably of at least 92%, still even more preferably of at least 95%, most preferably of at least 97%, with a sequence of the same length contained at the very 3′ end or at the very 5′ end of its reference template sequence or conservative sub-fragment thereof, or of the sequence that is fully complementary to said reference template sequence or conservative sub-fragment thereof over the entire length of this reference template sequence or complementary sequence thereof.

For example, a primer pair of the invention may consist of a forward primer and a reverse primer, which each independently consists of 14-30 nucleotides,

wherein the sequence of the forward primer has an identity of at least 80%, preferably of at least 85%, more preferably of at least 90%, even more preferably of at least 92%, still even more preferably of at least 95%, most preferably of at least 97%, with a sequence of the same length contained at the very 5′ end of its reference template sequence or conservative sub-fragment thereof, and

wherein the sequence of the reverse primer has an identity of at least 80%, preferably of at least 85%, more preferably of at least 90%, even more preferably of at least 92%, still even more preferably of at least 95%, most preferably of at least 97%, with a sequence of the same length contained at the very 5′ end of the sequence that is fully complementary to said reference template sequence or conservative sub-fragment thereof, over the entire length of said reference template sequence or complementary sequence thereof.

A primer of the invention preferably consists of 14-28, more preferably of 15-28, even more preferably of 16-27, still even more preferably of 16-26, most preferably of 17-25 nucleotides, e.g., of 17, 18, 19, 20, 21, 22, 23, 24 or 25 nucleotides.

Said at least two CT-targeted primers preferably are one oligonucleotide of SEQ ID NO: 7 (forward primer), or a conservative variant thereof, and one oligonucleotide of SEQ ID NO: 12 (reverse primer), or a conservative variant thereof.

Said at least two MG-targeted primers preferably are:
one oligonucleotide of SEQ ID NO: 15 (forward primer), or a conservative variant thereof, and one oligonucleotide of SEQ ID NO:18 (reverse primer), or a conservative variant thereof, or are
at least one oligonucleotide selected from the group consisting of SEQ ID NO: 21;

27; 33; 39, or a conservative variant thereof, and at least one oligonucleotide selected from the group consisting of SEQ ID NO: 24; 30; 36, or a conservative variant thereof.

Said at least two MG-targeted primers can for example be at least one oligonucleotide selected from the group consisting of SEQ ID NO: 21; 27, or a conservative variant thereof, and at least one oligonucleotide selected from the group consisting of SEQ ID NO: 24; 30; 36, or a conservative variant thereof.

Said at least two MG-targeted primers preferably are one oligonucleotide of SEQ ID NO: 21, or a conservative variant thereof, and at least one oligonucleotide selected from the group consisting of SEQ ID NO: 24; 36, or a conservative variant thereof.

More preferably, said at least two MG-targeted primers are one oligonucleotide of SEQ ID NO: 21, or a conservative variant thereof and one oligonucleotide of SEQ ID NO: 24, or a conservative variant thereof.

More preferably, said at least two MG-targeted primers are one oligonucleotide of SEQ ID NO: 27, or a conservative variant thereof, and at least one oligonucleotide selected from the group consisting of SEQ ID NO: 24; 30; 36, or a conservative variant thereof. Most preferably, said at least two MG-targeted primers are one oligonucleotide of SEQ ID NO: 27, or a conservative variant thereof, and one oligonucleotide of SEQ ID NO: 30, or a conservative variant thereof.

Said at least two MG-targeted primers preferably are one oligonucleotide of SEQ ID NO: 36, or a conservative variant thereof, and at least one oligonucleotide selected from the group consisting of SEQ ID NO: 21; 27; 33 and 39, or a conservative variant thereof.

Most preferably, said at least two MG-targeted primers are one oligonucleotide of SEQ ID NO: 33 and one oligonucleotide of SEQ ID NO: 36; or are one of SEQ ID NO: 39 and one oligonucleotide of SEQ ID NO: 36.

Said at least two NG-targeted primers preferably are one oligonucleotide of SEQ ID NO: 44, or a conservative variant thereof, and one oligonucleotide of SEQ ID NO: 47, or a conservative variant thereof.

Conservative variants of such primers more particularly comprise those variant primers, the sequence of which has an identity of at least 80%, preferably of at least 85%, more preferably of at least 90%, even more preferably of at least 92%, still even more preferably of at least 95%, most preferably of at least 97%, with at least one of the above-mentioned SEQ ID primer sequences.

Said at least one CT- or MG- or NG-targeted probe, which is suitable for the detection of CT or MG or NG, respectively, in real-time multiplex amplification is an oligonucleotide, the sequence of which is suitable for use as a probe for the detection of at least one amplicon produced from CT or MG or NG, respectively, by said at least two CT- or MG- or NG-targeted primers.

The sequence of such a CT- or MG- or NG-targeted probe hence necessarily differs from the sequence of the CT-, MG-, NG-targeted primers.

Advantageously, said at least one CT-targeted probe is a CT-specific probe that anneals to CT amplicons, without annealing to NG or MG amplicons under conditions of at least moderate stringency.

Advantageously, said at least one MG-targeted probe is a MG-specific probe that anneals to MG amplicons, without annealing to CT or NG amplicons under conditions of at least moderate stringency.

Advantageously, said at least one NG-targeted probe is a NG-specific probe that anneals to NG amplicons, without annealing to CT or MG amplicons under conditions of at least moderate stringency.

Said at least one CT-specific probe preferably is an oligonucleotide of 15-60 nucleotides, which is sufficiently complementary to a fragment of the same size of said CT reference template sequence, or to a fragment of the same size of the sequence that is fully complementary to said CT reference template sequence over the entire length of said reference template sequence, to anneal to said CT reference template sequence or complementary sequence thereof, under conditions of at least moderate stringency,

but which is not sufficiently complementary to any fragment of the same size of said MG or NG reference template sequence, or of the sequence that is fully complementary to said MG or NG reference template sequence over the entire length of said MG or NG reference template sequence, to anneal to said MG or NG reference template sequence or complementary sequence thereof, under the same stringency conditions.

Said at least one MG-specific probe preferably is an oligonucleotide of 15-60 nucleotides, which is sufficiently complementary to a fragment of the same size of said MG reference template sequence, or to a fragment of the same size of the sequence that is fully complementary to said MG reference template sequence over the entire length of said reference template sequence, to anneal to said MG reference template sequence or complementary sequence thereof, under conditions of at least moderate stringency,

but which is not sufficiently complementary to any fragment of the same size of said CT or NG reference template sequence, or of the sequence that is fully complementary to said CT or NG reference template sequence over the entire length of said CT or NG reference template sequence, to anneal to said CT or NG reference template sequence or complementary sequence thereof, under the same stringency conditions.

Said at least one NG-specific probe preferably is an oligonucleotide of 15-60 nucleotides, which is sufficiently complementary to a fragment of the same size of said NG reference template sequence, or to a fragment of the same size of the sequence that is fully complementary to said NG reference template sequence over the entire length of said reference template sequence, to anneal to said NG reference template sequence or complementary sequence thereof, under conditions of at least moderate stringency,

but which is not sufficiently complementary to any fragment of the same size of said CT or MG reference template sequence, or of the sequence that is fully complementary to said MG or NG reference template sequence over the entire length of said CT or MG reference template sequence, to anneal to said CT or MG reference template sequence or complementary sequence thereof, under the same stringency conditions.

More preferably, said at least one CT-specific probe is a fragment of at least 15 nucleotides of said CT reference template sequence, or of the sequence that is fully complementary to said CT reference template sequence over the entire length of said reference template sequence,

wherein said fragment does not anneal to said MG or NG reference template sequences under conditions of at least moderate stringency.

More preferably, said at least one MG-specific probe is a fragment of at least 15 nucleotides of said MG reference template sequence, or of the sequence that is fully complementary to said MG reference template sequence over the entire length of said reference template sequence,

wherein said fragment does not anneal to said CT or NG reference template sequences under conditions of at least moderate stringency.

More preferably, said at least one NG-specific probe is a fragment of at least 15 nucleotides of said NG reference template sequence, or of the sequence that is fully complementary to said NG reference template sequence over the entire length of said reference template sequence,

wherein said fragment does not anneal to said CT or MG reference template sequences under conditions of at least moderate stringency.

The expression “conditions of at least moderate stringency” is intended to mean conditions of moderate, high or very high stringency.

The expressions “moderate stringency”, “high stringency” and “very high stringency” are given their ordinary meaning in the field.

Stringency refers to hybridization conditions chosen to optimize binding of polynucleotide sequences with different degrees of complementarity. Stringency is affected by factors such as temperature, salt conditions, the presence of organic solvents in the hybridization mixtures, and the lengths and base compositions of the sequences to be hybridized and the extent of base mismatching, and the combination of parameters is more important than the absolute measure of any one factor.

Illustrative conditions of moderate stringency comprise:
hybridization to filter-bound DNA in 5×SSC, 2% sodium dodecyl sulfate (SDS), 100 microgrammes/mL single stranded DNA at 55-65° C. for 8 hours, and washing in 0.2×SSC and 0.2% SDS at 50-55° C. for thirty minutes.

Illustrative conditions of high stringency comprise:
hybridization to filter-bound DNA in 5×SSC, 2% sodium dodecyl sulfate (SDS), 100 microgrammes/mL single stranded DNA at 55-65° C. for 8 hours, and washing in 0.2×SSC and 0.2% SDS at 60-65° C. for thirty minutes.

Illustrative conditions of very high stringency comprise:
hybridization to filter-bound DNA in 5×SSC, 2% sodium dodecyl sulfate (SDS), 100 microgrammes/mL single stranded DNA at 55-65° C. for 8 hours, and washing in 0.1×SSC and 0.1% SDS at 60-65° C. for thirty minutes.

Most preferably, said at least one CT-specific probe, which is suitable for the detection of CT in real-time multiplex amplification, preferably is:
i. a fragment of at least 15 nucleotides of said CT reference template sequence, or of the sequence that is fully complementary to said CT reference template sequence over the entire length of said reference template sequence, or
ii. a conservative variant thereof, which derives from a fragment of i. by deletion and/or substitution and/or addition of at least one nucleotide, but which has retained the capacity of being a CT-specific probe, wherein said fragment does not anneal to said NG or MG reference template sequences, e.g., a conservative variant of a fragment of i., the sequence of which has at least 90% identity with a fragment of i. over the entire length of said fragment of i.

Most preferably, said at least one MG-specific probe, which is suitable for the detection of MG iii real-time multiplex amplification, preferably is:
iii. a fragment of at least 15 nucleotides of said MG reference template sequence, or of the sequence that is fully complementary to said MG reference template sequence over the entire length of said reference template sequence, or
iv. a conservative variant thereof, which derives from a fragment of i. by deletion and/or substitution and/or addition of at least one nucleotide, but which has retained the capacity of being a MG-specific probe, wherein said fragment does not anneal to said CT or NG reference template sequences, e.g., a conservative variant of a fragment of iii., the sequence of which has at least 90% identity with a fragment of iii. over the entire length of said fragment of iii.

Most preferably, said at least one NG-specific probe, which is suitable for the detection of NG in real-time multiplex amplification, preferably is:
v. a fragment of at least 15 nucleotides of said NG reference template sequence, or of the sequence that is fully complementary to said NG reference template sequence over the entire length of said reference template sequence, or
vi. a conservative variant thereof, which derives from a fragment of i. by deletion and/or substitution and/or addition of at least one nucleotide, but which has retained the capacity of being a NG-specific probe, wherein said fragment does not anneal to said CT or MG reference template sequences, e.g., a conservative variant of a fragment of v., the sequence of which has at least 90% identity with a fragment of v. over the entire length of said fragment of v. (global alignment, also referred to as “needle” alignment).

Said percentage of identity preferably is of at least 91%, more preferably of at least 92%, even more preferably of at least 93%, still more preferably of at least 94%, most preferably of at least 95%, e.g., 95%, at least 96%, at least 97%, at least 98%, or at least 99%.

A probe of the invention comprises at least 15 nucleotides. For example, it consists of 15-60 nucleotides. A probe of the invention preferably consists of 15-50, more preferably of 15-40, even more preferably of 15-30, still more preferably of 16-30, even still more preferably of 18-30, most preferably of 19-30, still most preferably of 21-29, even still most preferably of 22-27 nucleotides, e.g., 22, 23, 24, 25, 26 or 27 nucleotides.

Said at least one CT-specific probe preferably is of SEQ ID NO: 8 or 10 (CT probes SCT 175b and 175c), or is the sequence that is fully complementary to this SEQ ID sequence, over the entire length of this SEQ ID sequence.

Said at least one MG-specific probe preferably is selected from the group consisting of SEQ ID NO: 16; 22; 28; 34 and 40 (MG probes SF-MG 258c, MGBR 140c, MGBR 186j, MGBR 178q, MGBR 204u), or is the sequence that is fully complementary to this SEQ ID sequence, over the entire length of this SEQ ID sequence.

Said at least one NG-specific probe is of SEQ ID NO: 45 (NG probe pilEc), or is the sequence that is fully complementary to this SEQ ID sequence, over the entire length of this SEQ ID sequence.

A probe of the invention can be linked to at least one detection label, and/or at least one nucleotide arm that is unrelated to CT, MG and NG and that is intended to carry a quencher or a reporter (e.g., a fluorophore).

Various formats (types) of probes, including Taqman™ probes (hydrolysis probes), molecular Beacons™ (beacon probes or molecular beacon probes), and Scorpion™ probes are known in the art.

It may e.g., be linked to at least one beacon arm, or to at least one Scorpion™ arm, preferably at least one of such arms in 5′ and/or 3′, most preferably two of such arms, in 5′ and in 3′, respectively.

One of preferred formats is the beacon format.

The structure of molecular beacons is as follows. A short nucleotide sequence (so-called beacon arm) which is unrelated to the target sequence is thus covalently linked to both ends of the probe. A short unrelated arm is thus linked in 5′ of the probe, and is labelled with a fluorescent moiety (i.e. fluorescent dye or fluorescent marker). Another but still unrelated arm is linked to the 3′ end of probe and is labelled with a fluorescence quenching moiety. Thus, molecular beacons have a fluorophore and a quencher at opposite ends. The 5′ short arm is totally complementary to the one in 3′ so that they can anneal together, and thus can assume a hairpin structure when unhybridized to the target in solution. In this hairpin conformation, the quencher and the fluorescent dye are close enough to each other to allow efficient quenching of the fluorophore. However, when the probe encounters a target molecule, annealing is favoured with respect to the hairpin conformation when values of beacon arm Tm and probe Tm are suitably chosen (theoretically: probe Tm>beacon arm Tm>primer Tm, wherein Tm is the melting temperature of interest). The fluorophore and quencher move away from each other and the fluorophore can then fluoresce when illuminated by suitable light excitation. As PCR proceeds, amplification product accumulates, and the amount of fluorescence at any given cycle depends on the amount of amplification product present at that time. (See e.g., Sanjay Tyagi and Fred Russell Kramer, Nature Biotechnology 1996, volume 14, pages 303-308; Nature Biotechnology 1998, volume 16, pages 49-53).

(Remark: It is also possible to link the fluorophore at the 3′ end, while attaching the quencher at the 5′ end.)

Schematically, said probe can have the following formulae (molecular beacon format):

5′ Fluorophore-(arm1)-probe-(arm2)-Quencher 3′

5′ Quencher-(arm1)-probe-(arm2)-Fluorophore 3′

wherein arm1 and arm2 can be any short nucleotide sequences, e.g. in the range of 3-10 nucleotides, preferably 5, 6, 7 nucleotides, allowing for the hair pin structure formation under suitable stringency conditions, i.e. arm1 and arm2 are totally complementary to anneal under the desired stringency conditions (standard PCR stringency conditions include, for example, an annealing temperature of 55 to 65° C. and an Mg concentration of 4 to 8 mM). However, arm1 and arm2 are unrelated to the target sequence of the probe, i.e. the hairpin conformation resulting from the annealing between arm1 and arm2 is essentially the only possible secondary structure for the probe when unhybridized. The skilled person would know how to choose such arms for a given probe.

Illustrative beacon arms are given in example 1 below.

By fluorophore, it is herein understood any fluorescent marker/dye known in the art. Examples of such suitable fluorescent markers include Fam, Hex, Tet, Joe, Rox, Tamra, Max, Edans, Cy dyes such as Cy5, Fluorescein, Coumarin, Eosine, Rhodamine, Bodipy, Alexa, Cascade Blue, Yakima Yellow, Lucifer Yellow and Texas Red (all of them are Trade-Marks), the family of ATTO dyes.

By quencher, we herein understand any quencher known in the art. Examples of such quenchers include Dabcyl, Dark Quencher, Eclipse Dark Quencher, ElleQuencher, Tamra, BHQ and QSY (all of them are Trade-Marks).

The skilled person would know which combinations of dye/quencher are suitable when designing a probe.

In a preferred embodiment according to the invention, spectral properties of said probes can be chosen as to not interfere with each other. In particular, when probes are used in multiplex, each single probe can have its own fluorophore being spectrally significantly different from each other, i.e., the absorption/emission spectra are essentially non-overlapping. This advantageously allows for low-noise multiplex detection for all single probes, making sure that individual signals do not interfere with each other in detection.

Examples of dyes which can be used together in multiplex include Fam with Tamra, Fam with Tamra with Texas Red.

The choice of appropriate dyes to be used together may also be dependent of the filter contained in the amplification apparatus.

Said at least one CT-specific probe most preferably is of SEQ ID NO: 9 or 11 (CT probes SCT 175b and 175c with beacon arms), or the sequence that is fully complementary to this SEQ ID sequence, over the entire length of this SEQ ID sequence.

Said at least one MG-specific probe most preferably is selected from the group consisting of SEQ ID NO: 17; 23; 29; 35 and 41 (MG probes SF-MG 258c, MGBR 140c, MGBR 186j, MGBR 178q, MGBR 204u with beacon arms), or is the sequence that is fully complementary to this SEQ ID sequence, over the entire length of this SEQ ID sequence.

Said at least one NG-specific probe most preferably is of SEQ ID NO: 46 (NG probe pilEc with beacon arms), or is the sequence that is fully complementary to this SEQ ID sequence, over the entire length of this SEQ ID sequence.

Said at least one CT- or MG- or NG-specific probe may also be an oligonucleotide, which is a conservative variant of said probe SEQ ID, as above-described, i.e., which derives from said SEQ ID sequence by deletion and/or substitution and/or addition of at least one nucleotide, but which has retained the capacity of being a CT- or MG- or NG-specific probe, e.g., a conservative variant of said SEQ ID sequence, the sequence of which has at least 90% identity with said SEQ ID sequence, over the entire length of said SEQ ID sequence (global alignment, also referred to as “needle” alignment).

One of the special technical features shared by said CT, MG and NG reference template sequences is that they are reference template sequences, which are suitable for construct and produce CT-, MG- and NG-targeted primers, as well as CT-, MG- and NG-specific probes, which can be used together in multiplex in the same tube to specifically detect at least one CT and/or at least one MG and/or at least one NG, advantageously at least one CT and at least one MG and at least one NG, in real-time time in said same tube.

Preferably, said at least two CT-targeted primers are one oligonucleotide of SEQ ID NO: 7 and one oligonucleotide of SEQ ID NO: 12, and said at least one CT-specific probe is selected from the group consisting of SEQ ID NO: 8; 9; 10; 11.

Preferably, said at least two MG-targeted primers are one oligonucleotide of SEQ ID NO: 15 and one oligonucleotide of SEQ ID NO: 18, and said at least one MG-specific probe is of SEQ ID NO: 16 or 17, or is the sequence that is fully complementary to this SEQ ID sequence, over the entire length of this SEQ ID sequence.

Preferably, said at least two MG-targeted primers are one oligonucleotide of SEQ ID NO: 21 and one oligonucleotide of SEQ ID NO: 24, and said at least one MG-specific probe is of SEQ ID NO: 22 or 23, or is the sequence that is fully complementary to this SEQ ID sequence, over the entire length of this SEQ ID sequence.

Preferably, said at least two MG-targeted primers are one oligonucleotide of SEQ ID NO: 27 and one oligonucleotide of SEQ ID NO: 30, and said at least one MG-specific probe is of SEQ ID NO: 28 or 29, or is the sequence that is fully complementary to this SEQ ID sequence, over the entire length of this SEQ ID sequence.

Preferably, said at least two MG-targeted primers are one oligonucleotide of SEQ ID NO: 33 and one oligonucleotide of SEQ ID NO: 36, and said at least one MG-specific probe is of SEQ ID NO: 34 or 35, or is the sequence that is fully complementary to this SEQ ID sequence, over the entire length of this SEQ ID sequence.

Preferably, said at least two MG-targeted primers are one oligonucleotide of SEQ ID NO: 39 and one oligonucleotide of SEQ ID NO: 36, and said at least one MG-specific probe is of SEQ ID NO: 40 or 41, or is the sequence that is fully complementary to this SEQ ID sequence, over the entire length of this SEQ ID sequence.

Preferably, said at least two NG-targeted primers are one oligonucleotide of SEQ ID NO: 44 and one oligonucleotide of SEQ ID NO: 47, and said at least one NG-specific probe is of SEQ ID NO: 45 or 46, or is the sequence that is fully complementary to this SEQ ID sequence, over the entire length of this SEQ ID sequence.

Advantageously, in accordance with the invention, said amplification primers can comprise at least two CT-targeted primers, and at least two MG-targeted primers, and at least two NG-targeted primers.

Preferably:
said at least two CT-targeted primers are one oligonucleotide of SEQ ID NO: 7 and one oligonucleotide of SEQ ID NO: 12, and
said at least two MG-targeted primers are one oligonucleotide of SEQ ID NO: 15 and one oligonucleotide of SEQ ID NO: 18; or one oligonucleotide of SEQ ID NO: 21 and one oligonucleotide of SEQ ID NO: 24; or one oligonucleotide of SEQ ID NO: 27 and one oligonucleotide of SEQ ID NO: 30; or one oligonucleotide of SEQ ID NO: 33 and one oligonucleotide of SEQ ID NO: 36; or one oligonucleotide of SEQ ID NO: 39 and one oligonucleotide of SEQ ID NO: 36, and
said at least two NG-targeted primers are one oligonucleotide of SEQ ID NO: 44 and one oligonucleotide of SEQ ID NO: 47.

Advantageously, in accordance with the invention, said at least one probe can comprise at least one CT-specific probe and at least one MG-specific probe and at least one NG-specific.

Preferably:
said at least two CT-targeted primers are one oligonucleotide of SEQ ID NO: 7 and one oligonucleotide of SEQ ID NO: 12, and said at least one CT-specific probe is of SEQ ID NO: 8, 9, 10 or 11, or is the sequence that is fully complementary to this SEQ ID sequence, over the entire length of this SEQ ID sequence, and
said at least two NG-targeted primers are one oligonucleotide of SEQ ID NO: 44 and one oligonucleotide of SEQ ID NO: 47, and said at least one NG-specific probe is of SEQ ID NO: 45 or 46, or is the sequence that is fully complementary to this SEQ ID sequence, over the entire length of this SEQ ID sequence; and
said at least two MG-targeted primers and said at least one MG-specific probe are as follows:
   said at least two MG-targeted primers are one oligonucleotide of SEQ ID NO: 15 and one oligonucleotide of SEQ ID NO: 18, and said at least one MG-specific probe is of SEQ ID NO: 16 or 17, or is the sequence that is fully complementary to this SEQ ID sequence, over the entire length of this SEQ ID sequence; or
   said at least two MG-targeted primers are one oligonucleotide of SEQ ID NO: 21 and one oligonucleotide of SEQ ID NO: 24, and said at least one MG-specific probe is of SEQ ID NO: 22 or 23, or is the sequence that is fully complementary to this SEQ ID sequence, over the entire length of this SEQ ID sequence; or
   said at least two MG-targeted primers are one oligonucleotide of SEQ ID NO: 27 and one oligonucleotide of SEQ ID NO: 30, and said at least one MG-specific probe is of SEQ ID NO: 28 or 29, or is the sequence that is fully complementary to this SEQ ID sequence, over the entire length of this SEQ ID sequence; or
   said at least two MG-targeted primers are one oligonucleotide of SEQ ID NO: 33 and one oligonucleotide of SEQ ID NO: 36, and said at least one MG-specific probe is of SEQ ID NO: 34 or 35, or is the sequence that is fully complementary to this SEQ ID sequence, over the entire length of this SEQ ID sequence; or
   said at least two MG-targeted primers are one oligonucleotide of SEQ ID NO: 39 and one oligonucleotide of SEQ ID NO: 36, and said at least one MG-specific probe is of SEQ ID NO: 40 or 41, or is the sequence that is fully complementary to this SEQ ID sequence, over the entire length of this SEQ ID sequence.

In accordance with an advantageous embodiment of the invention, the detection of CT and/or MG and/or NG can be made in real-time multiplex amplification.

Of course, the primers and probes of the invention are also suitable for other protocols, including simplex protocols, multiplex protocols, end-point protocols, qualitative protocols, quantitative protocols, combinations thereof, and the like.

Said amplification can be any nucleic acid amplification, which is found appropriate to the skilled person, for example a PCR (Polymerase Chain Reaction), or an isothermal amplification technique, e.g., TMA (transcription mediated amplification), NASBA (nucleic acid sequence based amplification), 3SR (self sustained sequence replication) or strand displacement amplification.

Said amplification preferably is PCR.

In a preferred embodiment, the primers according to the invention are used in a final concentration range 20-2000 nM. Typically, said primers can be used at a final concentration range of 20-1300 nM, preferably of 20-1250 nM, more preferably of 25-1250 nM, e.g., of about 25, 125, 250, 500, 850, 1250 nM.

Probe concentration in a PCR reaction can be optimized, typically by varying the final concentration from 50 nM to 1000 nM. In a preferred embodiment, each probe according to the invention is used at a final concentration range of 75-300 nM, preferably 75-250 nM, more preferably 100-250 nM, even more preferably 150-200 nM, e.g., of about 150 nM or about 200 nM.

Appropriate amplification conditions are known to those skilled in the art. They include temperature conditions, in particular thermal cycling conditions, e.g., temperature, duration, number, heating rate of the cycles. In a preferred embodiment, said temperature conditions include conditions suitable for a PCR. In another preferred embodiment, said conditions include conditions suitable for a Q-PCR.

In accordance with the invention, an internal control (IC), which is unrelated to CT and/or MG and/or NG can also be implemented, such as the IC of SEQ ID NO: 48.

An appropriate primer pair for amplification of said IC comprises the primer pair of SEQ ID NO: 49 and 52. An appropriate IC probe comprises the probe of SEQ ID NO: 50 (SEQ ID NO: 51 in beacon format).

These IC, IC primers and IC probes are also encompassed individually as such by the application.

A 16S rRNA primer pair may also be implemented, such as, e.g., the primer pair of SEQ ID NO: 53 and 54. This system is used in order to detect the presence of bacterial DNA in sample.

The present application also relates to any amplicon obtainable by implementation of the process of the invention on a CT- and/or MG- and/or NG-containing sample.

The present application also relates to the primers and probes of the present invention, as such, i.e., as individual oligonucleotide products.

According to the invention, all the provided oligonucleotides can be either kept separately, or partially mixed, or totally mixed.

Said oligonucleotides can be provided under dry form, or solubilized in a suitable solvent, as judged by the skilled person. Suitable solvents include TE, PCR-grade water, and the like.

The application further relates to every product that is herein described, and more particularly to every reference template polynucleotide, to every primer and to every probe, as an individual product.

The application also relates to every possible combination that can be made of at least two products of the invention, preferably of at least three products of the invention, such as, e.g., of at least two primers of the invention and at least one probe of the invention.

The invention thus relates to a polynucleotide suitable for use as a reference template sequence in the design of primers and probes that can be used in the same tube for the detection of CT and MG and NG in real-time multiplex amplification, wherein said polynucleotide is selected from:
for the design of CT primers and probes: a fragment consisting of positions 5571-5760 (SEQ ID NO: 5) of the CT sequence of SEQ ID NO: 1 (CT pCHL1 plasmid; J03321), or of a conservative sub-fragment thereof, which has retained the property of being a suitable reference template sequence, to construct and produce CT-targeted primers, which allow for a real-time multiplex detection of CT, or a sequence that is fully complementary to said fragment or sub-fragment over the entire length of said fragment or sub-fragment,
for the design of MG primers and probes: a fragment consisting of:
   positions 1-270 (SEQ ID NO: 13) of the MG sequence of SEQ ID NO: 2 (5′ region of MG adhesin gene; X91074), or of a conservative sub-fragment thereof, which has retained the property of being a suitable reference template sequence, to construct and produce MG-targeted primers, which allow for a real-time multiplex detection of MG, or a sequence that is fully complementary to said fragment or sub-fragment over the entire length of said fragment or sub-fragment,
   positions 1140-1290 (SEQ ID NO: 19) of the MG sequence of SEQ ID NO: 3 (MG adhesion gene; M31431), or of a conservative sub-fragment thereof, which has retained the property of being a suitable reference template sequence, to construct and produce MG-targeted primers, which allow for a real-time multiplex detection of MG, or a sequence that is fully complementary to said fragment or sub-fragment over the entire length of said fragment or sub-fragment,
   positions 1060-1250 (SEQ ID NO: 25) of the MG sequence of SEQ ID NO: 3, or of a conservative sub-fragment thereof, which has retained the property of being a suitable reference template sequence, to construct and produce MG-targeted primers, which allow for a real-time multiplex detection of MG, or a sequence that is fully complementary to said fragment or sub-fragment over the entire length of said fragment or sub-fragment,
   positions 1520-1710 (SEQ ID NO: 31) of the MG sequence of SEQ ID NO: 3, or of a conservative sub-fragment thereof, which has retained the property of being a suitable reference template sequence, to construct and produce MG-targeted primers, which allow for a real-time multiplex detection of MG, or a sequence that is fully complementary to said fragment or sub-fragment over the entire length of said fragment or sub-fragment,
   positions 1500-1710 (SEQ ID NO: 37) of the MG sequence of SEQ ID NO: 3, or of a conservative sub-fragment thereof, which has retained the property of being a suitable reference template sequence, to construct and produce MG-targeted primers, which allow for a real-time multiplex detection of MG, or a sequence that is fully complementary to said fragment or sub-fragment over the entire length of said fragment or sub-fragment,
for the design of NG primers and probes: a fragment consisting of positions 101-380 (SEQ ID NO: 42) of the NG sequence of SEQ ID NO: 4 (NG pilE gene; AF042097), or of a conservative sub-fragment thereof, which has retained the property of being a suitable reference template sequence, to construct and produce NG-targeted primers, which allow for a real-time multiplex detection of NG, or a sequence that is fully complementary to said fragment or sub-fragment over the entire length of said fragment or sub-fragment.

Preferably, said reference template polynucleotide is:
for the design of CT primers and probes: the fragment consisting of positions 5580-5754 (SEQ ID NO: 6) of the CT sequence of SEQ ID NO:1, or a sequence that is fully complementary to said fragment over the entire length of said fragment,
for the design of MG primers and probes:
   the fragment consisting of positions 2-259 (SEQ ID NO: 14) of the MG sequence of SEQ ID NO:2, or a sequence that is fully complementary to said fragment or sub-fragment over the entire length of said fragment, or
   the fragment consisting of positions 1144-1283 (SEQ ID NO:20) of the MG sequence of SEQ ID NO:3, or a sequence that is fully complementary to said fragment or sub-fragment over the entire length of said fragment, or
   the fragment consisting of positions 1064-1249 (SEQ ID NO:26) of the MG sequence of SEQ ID NO:3, or a sequence that is fully complementary to said fragment or sub-fragment over the entire length of said fragment, or
   the fragment consisting of positions 1527-1704 (SEQ ID NO:32) of the MG sequence of SEQ ID NO:3, or a sequence that is fully complementary to said fragment or sub-fragment over the entire length of said fragment, or
   the fragment consisting of positions 1501-1704 (SEQ ID NO:38) of the MG sequence of SEQ ID NO:3, or a sequence that is fully complementary to said fragment or sub-fragment over the entire length of said fragment,
for the design of NG primers and probes: the fragment consisting of positions 114-365 (SEQ ID NO: 43) of the NG sequence of SEQ ID NO: 4, or a sequence that is fully complementary to said fragment or sub-fragment over the entire length of said fragment.

The application also relates to a primer, which is especially adapted to the detection of CT and/or MG and/or NG in real-time multiplex amplification, which is:
a CT-targeted primer, selected from the group consisting of SEQ ID NO: 7; 12, or
a MG-targeted primer, selected from the group consisting of SEQ ID NO: 15; 18; 21; 24; 27; 30; 33; 36; 39, or
a NG-targeted primer, selected from the group consisting of SEQ ID NO: 44; 47,
a conservative variant of such primers, as herein defined.

The application also relates to a primer system, which is especially adapted to the detection of CT and/or MG and/or NG in real-time multiplex amplification, which comprises at least two CT-targeted primers and/or at least two MG-targeted primers and/or at least two NG-targeted primers, such as:
at least one CT-targeted primer pair of SEQ ID NO: 7 and SEQ ID NO: 12, and/or
at least one MG-targeted primer pair selected from the following pairs: SEQ ID NO: 15 and 18; SEQ ID NO: 21 and 24; SEQ ID NO: 27 and 30; SEQ ID NO: 33 and 36; SEQ ID NO: 39 and 36, and/or
at least one NG-targeted primer pair of SEQ ID NO: 44 and 47.

The application also relates to a probe, which is especially adapted to the detection of CT and/or MG and/or NG in real-time multiplex amplification, which is:
a CT-specific probe of SEQ ID NO: 8; or 10, or which consists of a sequence that is fully complementary to this SEQ ID sequence, over the entire length of this SEQ ID sequence, or
a MG-specific probe of SEQ ID NO: 16; 22; 28; 34 or 40, or which consists of a sequence that is fully complementary to this SEQ ID sequence, over the entire length of this SEQ ID sequence, or
a NG-specific probe of SEQ ID NO: 45, or which consists of a sequence that is fully complementary to this SEQ ID sequence, over the entire length of this SEQ ID sequence, or
a conservative variant of such probes, as herein defined.

The application also relates to a beacon probe, which is especially adapted to the detection of CT and/or MG and/or NG in real-time multiplex amplification, which is:
a CT-specific probe of SEQ ID NO: 9 or 11, or which consists of a sequence that is fully complementary to this SEQ ID sequence, over the entire length of this SEQ ID sequence, or
a MG-specific probe of SEQ ID NO: 17; 23; 29; 35 or 41, or which consists of a sequence that is fully complementary to this SEQ ID sequence, over the entire length of this SEQ ID sequence, or
a NG-specific probe of SEQ ID NO: 45 or 46, or which consists of a sequence that is fully complementary to this SEQ ID sequence, over the entire length of this SEQ ID sequence, or
a conservative variant of such probes.

The application also relates to a primer and probe system, which is especially adapted to the detection of CT and/or MG and/or NG in real-time multiplex amplification, which comprises at least one primer of the invention and at least one probe of the invention, preferably at least one primer system of the invention, and at least one probe system of the invention.

The application also relates to an amplicon, obtainable by amplification of at least one nucleic acid from CT and/or MG and/or NG, by means of at least one primer system of the invention.

The application also relates to an amplification composition, comprising at least one amplicon according to the invention.

The application also relates to a kit for the diagnosis of an infection by CT and/or MG and/or NG, which comprises:
at least one primer system of the invention, and/or
at least one probe of the invention,
optionally, instructions for the use thereof and/or nucleotides.

In the kit according to the invention, the oligonucleotides (primers, probes) can be either kept separately, or partially mixed, or totally mixed.

Said oligonucleotides can be provided under dry form, or solubilized in a suitable solvent, as judged by the skilled person. Suitable solvents include TE, PCR-grade water, and the like.

In a preferred embodiment, the kit according to the invention can also contain further reagents suitable for a PCR step.

Such reagents are known to those skilled in the art, and include water, like nuclease-free water, RNase free water, DNAse-free water, PCR-grade water; salts, like magnesium, potassium; buffers such as Tris; enzymes, including polymerases, such as Taq, Vent, Pfu (all of them Trade-Marks), activable polymerase, and the like; nucleotides like deoxynucleotides, dideoxunucleotides, dNTPs, dATP, dTTP, dCTP, dGTP, dUTP; other reagents, like DTT and/or RNase inhibitors; and polynucleotides like polyT, polydT, and other oligonucleotides, e.g., primers.

In another preferred embodiment, the kit according to the invention comprises PCR controls. Such controls are known in the art, and include qualitative controls, positive controls, negative controls, internal controls, quantitative controls, internal quantitative controls, as well as calibration ranges. The internal control for said PCR step can be a template which is unrelated to the target template in the PCR step. Such controls also may comprise control primers and/or control probes. For example, in the case of HPV detection, it is possible to use as an internal control, a polynucleotide chosen within a gene whose presence is excluded in a sample originating from a human body (for example, from a plant gene), and whose size and GC content is equivalent to those from the target sequence.

Illustrative internal controls comprise the IC of SEQ ID NO: 48. Appropriate IC primers thus comprise the primer of SEQ ID NO: 49 and the primer of SEQ ID NO: 50, which together form a primer pair capable of amplifying said IC of SEQ ID NO: 48. Appropriate IC probes comprise the probe of SEQ ID NO: 50 (or of SEQ ID NO: 51, in beacon format).

In a preferred embodiment, the kit according to the invention contains means for extracting and/or purifying nucleic acid from a biological sample, e.g., from urine. Such means are well known to those skilled in the art.

In a preferred embodiment, the kit according to the invention contains instructions for the use thereof. Said instructions can advantageously be a leaflet, a card, or the like. Said instructions can also be present under two forms: a detailed one, gathering exhaustive information about the kit and the use thereof, possibly also including literature data; and a quick-guide form or a memo, e.g., in the shape of a card, gathering the essential information needed for the use thereof.

The present invention also relates to all the medical, biological, pharmaceutical applications of the detection process of the invention, and/or of the primers and/or probes of the invention.

The present invention thus relates to a process for the diagnosis or prognosis of a CT and/or MG and/or NG infection, which comprises detecting CT and/or MG and/or NG with at least one primer of the invention.

The present invention also relates to a process for monitoring the efficiency of an anti-CT and/or anti-MG and/or anti-NG treatment or drug, or an anti-CT and/or anti-MG and/or anti-NG candidate treatment or drug, which comprises determining by the detection method of the invention whether said treatment, drug, candidate treatment or candidate drug induces the non-reoccurrence, non-persistence, disappearance, or a decrease in the presence of at least one CT and/or MG and/or NG, whereby a positive determination indicates that said treatment, drug, candidate treatment or candidate drug is efficient.

The present invention also relates to a method to produce an anti-CT and/or anti-MG and/or anti-NG drug, which comprises:

providing at least one anti-CT and/or anti-MG and/or anti-NG candidate drug,

administering said at least one candidate anti-CT and/or anti-MG and/or anti-NG drug to a cell culture or to a non-human animal, wherein said cell culture or animal is or comprises at least one CT and/or MG and/or NG, and

determining by the detection method of the invention whether said candidate anti-CT and/or anti-MG and/or anti-NG drug induces the regression or disappearance of said at least one CT and/or MG and/or NG,

whereby a positive determination indicates that said candidate drug is an efficient CT and/or MG and/or NG drug.

In the present application, start and end values of any described range are to be understood as comprised within said range, e.g., an expression such as “position X to Y of a sequence” describes a sequence extending from nucleotide in position X to nucleotide in position Y, wherein both nucleotide in position X and nucleotide in position Y are part of said sequence.

The term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the invention.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In the present application, the term “at least x” relating to a set or group of n elements (wherein x is different from zero, and n is a number that is higher than x), explicitly encompasses each value, which is comprises between x and n. For example, the term “at least one” relating to a group or set of six elements explicitly encompasses one, two, three, four, five and six of said elements, as well as at least two, at least three, at least four, at least five of said elements.

Each of the relevant disclosures of all references cited herein is specifically incorporated by reference. The following examples are offered by way of illustration, and not by way of limitation.

### Example 1

### Design of the Primers and Probes

1.1. Selection of Primers and Probe for C. trachomatis (CT)

As a source of appropriate CT targets, the inventors selected the cryptic plasmid, which is present in all C. trachomatis serovars. This plasmid is contained at 7-10 copies per genome.

Four different sequences of this plasmid are available from Genbank:
plasmid pCHL1, of serotype D strain GO/86 (accession J03321; 7502 bp),
plasmid pCTT1 (accession M 19487; 7496 bp), of serotype B,
plasmid pLGV440 (accession X06707; 7501 bp) of serovar L1,
plasmid CTPLAS75 (accession X07547.1; 7499 bp) of serovar L2.

There is less than 1% variation between these four sequences (Comanducci, et al., 1990, Plasmid, 23: 149-154).

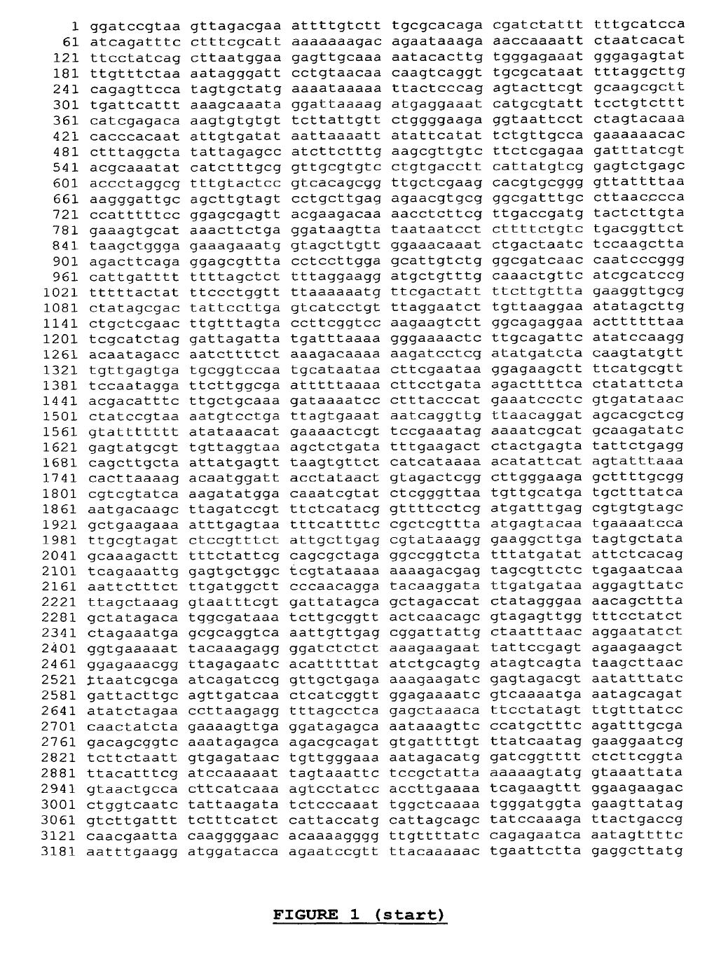

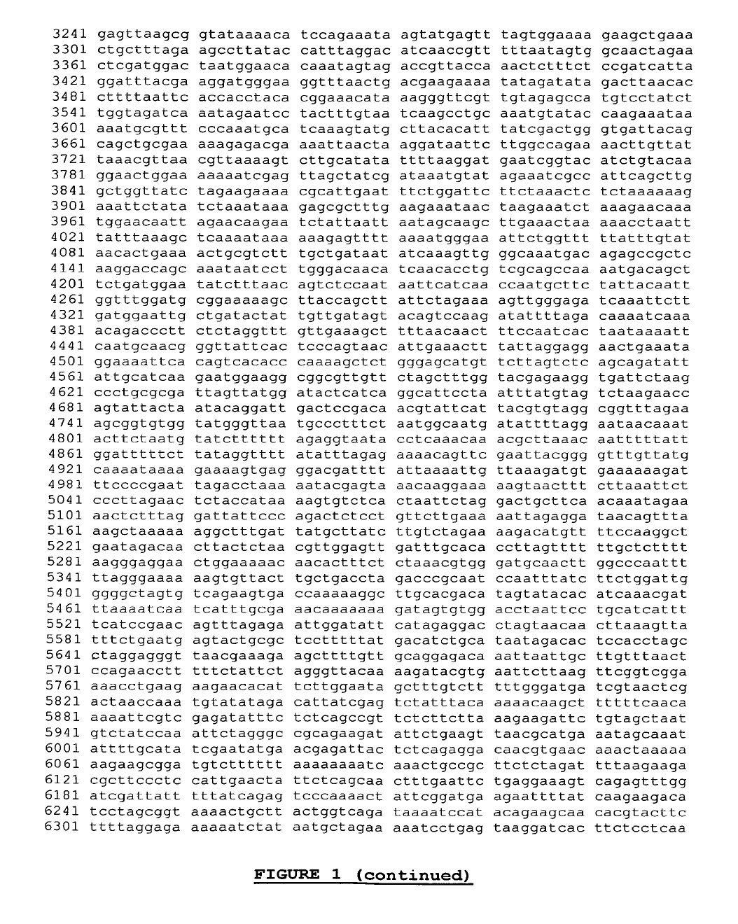

The sequence of plasmid pCHL1, which is available under accession number J03321, is shown on the enclosed FIG. 1 (SEQ ID NO: 1; 7502 nt).

A selected CT target sequence is located in ORF5 of pCHL1. A sub-sequence of SEQ ID NO: 5 has been selected by the inventors within the sequence of pCHL1 ORF5.

| CT sub-sequence selected within pCHL1 of SEQ ID NO: 1: | |
|---|---|
| SEQ ID NO: 5 | |
| 5571 | cttaaagtta |
| 5581 | tttctgaatg agtactgcgc tcctttttat ga(catctgca taatagacac tcca[ccta)gc |
| 5641 | ctaggagggt taacgaaaga a]gcttttgtt gcaggagaca aattaattgc ttgtttaact |
| 5701 | ccagaacctt tttctattct agggttacaa aagatacgtg aattcttaag ttcggtcgga |

Within this CT sub-sequence of SEQ ID NO: 5, a CT target sequence has been selected by the inventors (SEQ ID NO: 6). The forward and reverse primers are referred to as U-PC 5580 and L-PC 5754, respectively. Two FAM-labelled fluorescent probes (SCT175b, SCT175c) proved to be successful in detecting the amplicon. The sequences of these CT primers and probes are shown in the above CT sub-sequence of SEQ ID NO: 5 (from 5′ to 3′):
the sequence of a CT forward primer (U-PC 5580) (underlined and bold characters),
the respective sequences of two CT probes (probe SCT 175b in underlined and bold characters between parenthesis; probe SCT175c in underlined and bold characters between brackets), and
the target sequence of a CT reverse primer (L-PC 5754) (in underlined and bold characters).

The selected CT target sequence therefore is:

| CT target sequence (175nt): positions 5580 to 5754 of SEQ ID NO: 1 | |
|---|---|
| SEQ ID NO: 6 | |
| 5580 | a |
| 5581 | tttctgaatg agtactgcgc tcctttttat gacatctgca taatagacac tccacctagc |
| 5641 | ctaggagggt taacgaaaga agcttttgtt gcaggagaca aattaattgc ttgtttaact |
| 5701 | ccagaacctt tttctattct agggttacaa aagatacgtg aattcttaag ttcg |

CT forward primer U-PC 5580 (20nt): positions 5580 to 5599 of SEQ ID NO: 1
SEQ ID NO: 7
ATT TCT GAA TGA GTA CTG CG
CT probe SCT 175b (26 nt): positions 5613 to 5638 of SEQ ID NO: 1
SEQ ID NO: 8
CAT CTG CAT AAT AGA CAC TCC ACC TA
beacon ® arms: CGC GC in 5′; GC GCG in 3′
probe in beacon ® format:
(SEQ ID NO: 9)
CGC GCC ATC TGC ATA ATA GAC ACT CCA CCT AGC GCG
dye/quencher: FAM in 5′; Dabcyl in 3′
CT probe SCT 175c (27nt): positions 5635 to 5661 of SEQ ID NO: 1
SEQ ID NO: 10
CCT AGC CTA GGA GGG TTA ACG AAA GAA
beacon ® arms: ACG CGC in 5′; GCG CGT in 3′
probe in beacon ® format:
(SEQ ID NO: 11)
ACG CGC CCT AGC CTA GGA GGG TTA ACG AAA GAA GCG CGT
dye/quencher: FAM in 5′; Dabcyl in 3′
CT reverse primer L-PC 5754 (22nt): complementary to positions
5733 to 5754 of SEQ ID NO: 1
SEQ ID NO: 12
CGA ACT TAA GAA TTC ACG TAT C

1.2. Selection of Primers and Probe for Mycoplasma genitalium (MG)

The MG target sequence is selected within the gene coding for MgPa adhesin protein (major surface protein). This gene is referred to as adhesin gene, or Pa gene, or MgPa gene.

It is present at one copy per bacterium genome, and has been completely sequenced for reference strain G-37 (accession M31431).

A 5′ region of this gene has also been sequenced for four other MG strains; these sequences are available from GenBank:
accession X91074, strain M2341;
accession X91073, strain M2321;
accession X91071 strain 2288;
accession X91072, strain 2300.

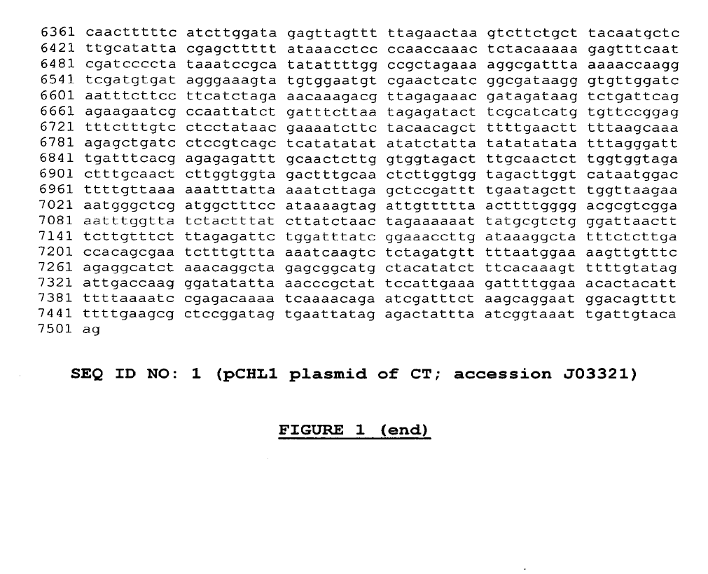

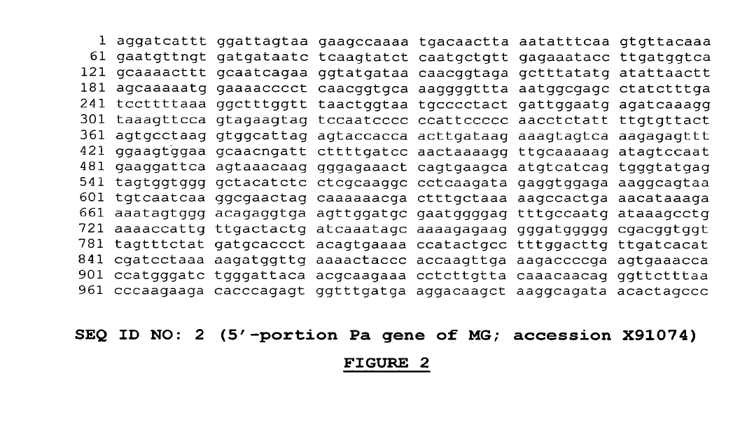

The sequence of the 5′ region of the adhesin gene of strain M2341, which is available under accession number X91074, is shown on the enclosed FIG. 2 (SEQ ID NO: 2).

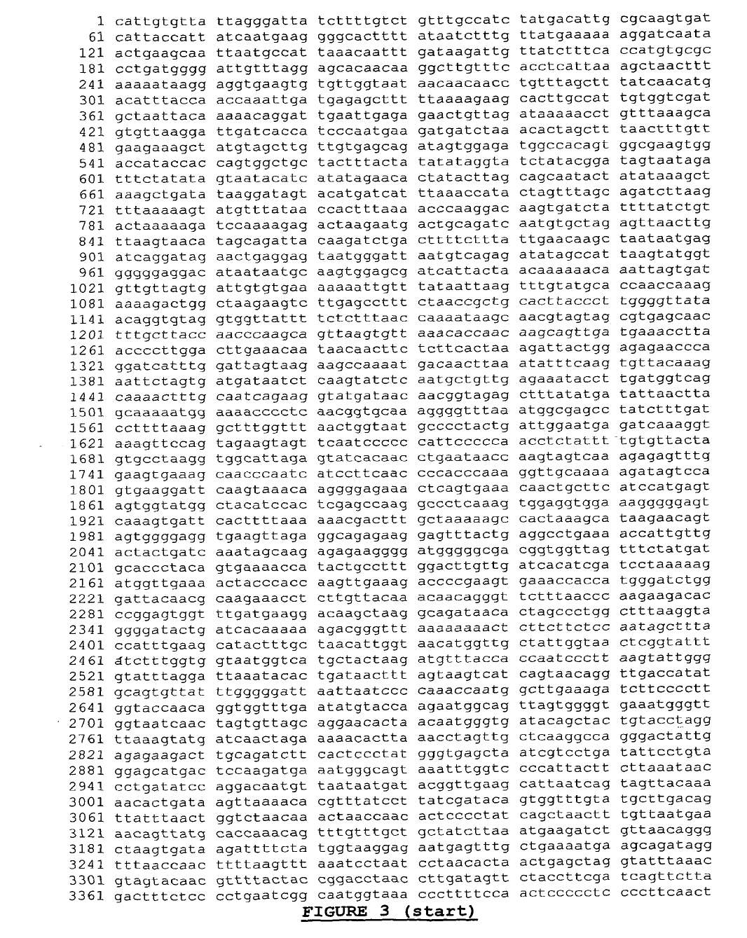

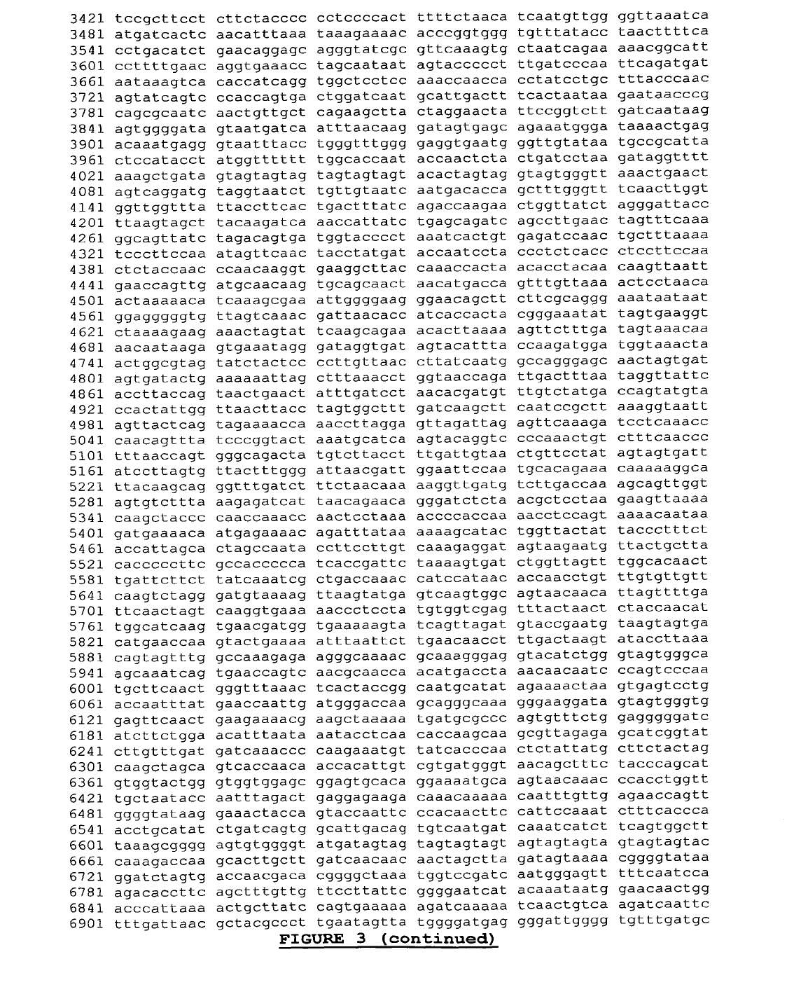

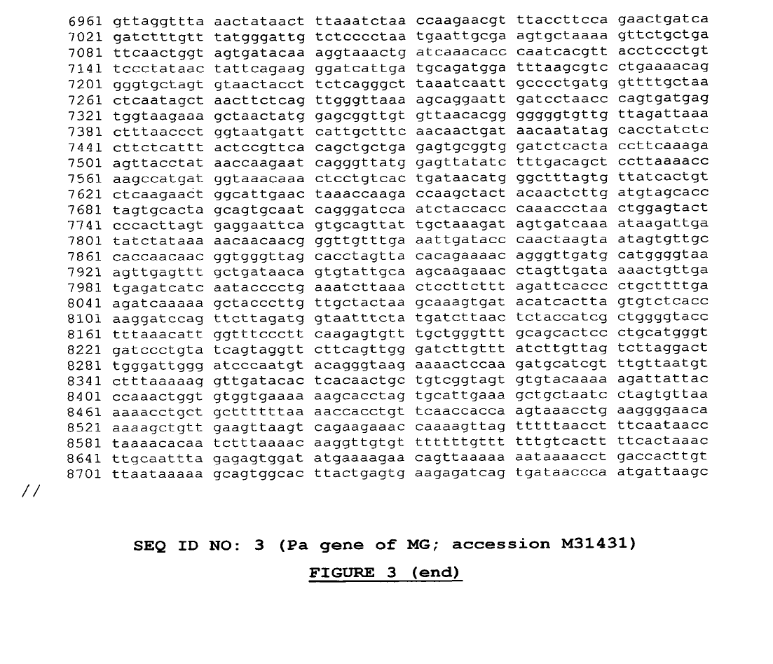

The sequence of the adhesin gene of strain G-37, which is available under accession M31431, is shown on the enclosed FIG. 3 (SEQ ID NO: 3).

### 1.2.1. Design on the MG Adhesin Gene 5′-Portion Having Accession Number X91074 (SEQ ID NO: 2):

A selected MG target sequence is located within the following Pa gene sub-sequence:

MG sub-sequence selected within MgPa sequence
of SEQ ID NO: 2:
SEQ ID NO: 13

| | |
|---|---|
| 1 | aggatcattt ggattagtaa gaagccaaaa tgacaactta |
| | aatatttcaa gtgttacaaa |
| 61 | gaatgttngt gatgataatc tcaagtatct caatgctgtt |
| | gagaaatacc ttgatggtca |
| 121 | gcaaaacttt gcaatcagaa ggtatgataa caacggtaga |
| | gctttatatg atattaactt |
| 181 | agcaaaaatg gaaaacccct caacggtgca aaggggttta |
| | aatggcgagc ctatctttga |
| 241 | tccttttaaa ggctttggtt taactggtaa |

A MG target sequence (SEQ ID NO: 14) has been selected within this MG sub-sequence of SEQ ID NO: 13. The sequence of a MG forward primer (U-MG 1320), the sequence of a MG probe (SF-MG 258c), and the target sequence of a MG reverse primer (L-MG 1578) are shown in underlined and bold characters within the above MG sub-sequence of SEQ ID NO: 13 (from 5′ to 3′, respectively).

The selected MG target sequence therefore is:

MG target sequence (258nt): positions 2 to 259 of SEQ ID NO: 2:
SEQ ID NO: 14
ggatcattt ggattagtaa gaagccaaaa tgacaactta aatatttcaa gtgttacaaa
gaatgttngt gatgataatc tcaagtatct caatgctgtt gagaaatacc ttgatggtca
gcaaaacttt gcaatcagaa ggtatgataa caacggtaga gctttatatg atattaactt
agcaaaaatg gaaaacccct caacggtgca aaggggttta aatggcgagc ctatctttga
tccttttaaa ggctttggt
MG forward primer (U-MG 1320; 24nt): positions 2 to 25 of SEQ
ID NO: 2
SEQ ID NO: 15
GGA TCA TTT GGA TTA GTA AGA AGC
MG probe (SF-MG 258c; 27nt): positions 136 to 162 of SEQ ID NO: 2
SEQ ID NO: 16
CAG AAG GTA TGA TAA CAA CGG TAG AGC
beacon ® arms: TGC GCA in 5′; TGC GCA in 3′
probe in beacon ® format:
(SEQ ID NO: 17)
TGC GCA CAG AAG GTA TGA TAA CAA CGG TAG AGC TGC GCA
dye/quencher: Tamra in 5′; Dabcyl in 3′
MG reverse primer (L-MG 1578; 23nt): complementary to positions
237 to 259 of SEQ ID NO: 2
SEQ ID NO: 18
ACC AAA GCC TTT AAA AGG ATC AA

### 1.2.2. Design on the MG Adhesin Gene Having Accession Number M31431 (SECO ID NO: 3):

Four other MG targets have been selected by the inventors. These four other MG targets are also located within the MG adhesin gene, more particularly within the 5′ region of this gene. They are defined with respect to the adhesin gene sequence, which is available under accession number M31431 (SEQ ID NO: 3, shown in FIG. 3).

These four other MG target sequences are located within the following MG sub-sequences:
positions 1140-1290 of SEQ ID NO: 3 (sub-sequence of SEQ ID NO: 19);
positions 1060-1250 of SEQ ID NO: 3 (sub-sequence of SEQ ID NO: 25);
positions 1520-1710 of SEQ ID NO: 3 (sub-sequence of SEQ ID NO: 31);
positions 1500-1710 of SEQ ID NO: 3 (sub-sequence of SEQ ID NO: 37).

| positions 1140-1290 of SEQ ID NO: 3 (sub-sequence of SEQ ID NO: 19): | |
|---|---|
| SEQ ID NO: 19 | |
| 1140 | a acaggtgtag gtggttattt tctctttaac caaaataagc aacgtagtag cgtgagcaac |
| 1201 | tttgcttacc aacccaagca gttaagtgtt aaacaccaac aagcagttga tgaaacctta |
| 1261 | accccttgga cttgaaacaa taacaacttc |
| positions 1060-1250 of SEQ ID NO: 3 (sub-sequence of SEQ ID NO: 25): | |
| SEQ ID NO: 25 | |
| 1060 | g tttgtatgca ccaaccaaag |
| 1081 | aaaagactgg ctaagaagtc ttgagccttt ctaaccgctg cacttaccct tggggttata |
| 1141 | acaggtgtag gtggttattt tctctttaac caaaataagc aacgtagtag cgtgagcaac |
| 1201 | tttgcttacc aacccaagca gttaagtgtt aaacaccaac aagcagttga |
| positions 1520-1710 of SEQ ID NO: 3 (sub-sequence of SEQ ID NO: 31): | |
| SEQ ID NO: 31 | |
| 1520 | c aacggtgcaa aggggtttaa atggcgagcc tatctttgat |
| 1561 | ccttttaaag gctttggttt aactggtaat gcccctactg attggaatga gatcaaaggt |
| 1621 | aaagttccag tagaagtagt tcaatccccc cattccccca acctctattt tgtgttacta |
| 1681 | gtgcctaagg tggcattaga gtatcacaac |
| positions 1500-1710 of SEQ ID NO: 3 (sub-sequence of SEQ ID NO: 37): | |
| SEQ ID NO: 37 | |
| 1500 | a gcaaaaatgg aaaacccctc aacggtgcaa aggggtttaa atggcgagcc tatctttgat |
| 1561 | ccttttaaag gctttggttt aactggtaat gcccctactg attggaatga gatcaaaggt |
| 1621 | aaagttccag tagaagtagt tcaatccccc cattccccca acctctattt tgtgttacta |
| 1681 | gtgcctaagg tggcattaga gtatcacaac |

### 1.2.2.1. Primer Pair (U-MG 1144/L-MG 1283), Amplicon of 140 bp:

MG target sequence (140 bp): positions 1144 to
1283 of SEQ ID NO: 3
SEQ ID NO: 20

| | |
|---|---|
| 1144 | ggtgtag gtggttattt tctctttaac caaaataagc |
| | aacgtagtag cgtgagcaac |
| 1201 | tttgcttacc aacccaagca gttaagtgtt aaacaccaac |
| | aagcagttga tgaaacctta |
| 1261 | accccttgga cttgaaacaa taa |

In bold and underlined characters, are shown (from 5′ to 3′) the sequences of the MG forward primer (U-MG1144), of the target for the MG probe (MGBR 140c), and of the target for the MG reverse primer (L-MG1283).

MG forward (U-MG 1144, 21 nt), positions 1144
to 1164 of SEQ ID NO: 3
SEQ ID NO: 21
GGT GTA GGT GGT TAT TTT CTC
MG probe (MGBR 140c Tamra/Dabcyl, 25 nt) positions
1208 to 1232 of SEQ ID NO: 3
SEQ ID NO: 22
ACC AAC CCA AGC AGT TAA GTG TTA A
beacon ® arms: CGCGTT in 5′; A ACG CG in 3′
probe in beacon ® format:
(SEQ ID NO: 23)
CGCGTT AC CAA CCC AAG CAG TTA AGT GTT AA A ACG
CG
dye/quencher: Tamra in 5′; Dabcyl in 3′
MG reverse primer (L-MG 1283, 22 nt):
complementary to positions 1262 to 1283 of
SEQ ID NO: 3
SEQ ID NO: 24
TTA TTG TTT CAA GTC CAA GGG G

### 1.2.2.2. Primer Pair (U MG 1087/L MG1249), Amplicon of 186 bp:

MG target sequence (186 bp): positions 1064 to
1249 of SEQ ID NO: 3
SEQ ID NO: 26

| | |
|---|---|
| 1064 | gt atgca ccaaccaaag |
| 1081 | aaaagactgg ctaagaagtc ttgagccttt ctaaccgctg |
| | cacttaccct tggggttata |
| 1141 | acaggtgtag gtggttattt tctctttaac caaaataagc |
| | aacgtagtag cgtgagcaac |
| 1201 | tttgcttacc aacccaagca gttaagtgtt aaacaccaac |
| | aagcagttg |

In bold and underlined characters, are shown (from 5′ to 3′) the sequences of the MG forward primer (U-MG1087), of the target for the MG probe (MGBR 186j Tamra/Dabcyl), and of the target for the MG reverse primer (L-MG1249).

MG forward (U-MG 1087, 24 nt), positions 1064
to 1087 of SEQ ID NO: 3
SEQ ID NO: 27
GTATGCACCAACCAAAGAAAAGAC
MG probe (MGBR 186j Tamra/Dabcyl, 28 nt) positions
1142 to 1168 of SEQ ID NO: 3
SEQ ID NO: 28
CAG GTG TAG GTG GTT ATT TTC TCT TTA
beacon ® arms: CGC GTT in 5′; AAC GCG in 3′
probe in beacon ® format:
SEQ ID NO: 29
CGC GTT CAG GTG TAG GTG GTT ATT TTC TCT
TTA AAC GCG
dye/quencher: Tamra in 5′; Dabcyl in 3′
MG reverse primer (L-MG 1249, 24 nt):
complementary to positions 1226 to 1249 of
SEQ ID NO: 3
SEQ ID NO: 30
CAA CTG CTT GTT GGT GTT TAA CAC

### 1.2.2.3. Primer Pair (U-MG 1527/L-MG 1704), Amplicon of 177 bp

MG target sequence (178 bp): positions 1527 to
1704 of SEQ ID NO: 3
SEQ ID NO: 32

| | |
|---|---|
| 1527 | gcaa aggggtttaa |
| | atggcgagcc tatctttgat |
| 1561 | ccttttaaag gctttggttt aactggtaat gcccctactg |
| | attggaatga gatcaaaggt |
| 1621 | aaagttccag tagaagtagt tcaatccccc cattccccca |
| | acctctattt tgtgttacta |
| 1681 | gtgcctaagg tggcattaga gtat |

In bold and underlined characters, are shown (from 5′ to 3′) the sequences of the MG forward primer (U-MG1527), of the target for the MG probe (MGBR 178q Tamra/Dabcyl), and of the target for the MG reverse primer (L-MG1704).

MG forward (U-MG 1527, 24 nt), positions 1527
to 1550 of SEQ ID NO: 3
SEQ ID NO: 33
GCA AAG GGG TTT AAA TGG CGA GCC
MG probe (MGBR 178q Tamra/Dabcyl, 26 nt) positions
1607 to 1630 of SEQ ID NO: 3
SEQ ID NO: 34
ATG AGA TCA AAG GTA AAG TTC CAG TA
beacon ® arms: AGC GTC in 5′; GAC GCT in 3′
probe in beacon ® format:
(SEQ ID NO: 35)
AGC GTC ATG AGA TCA AAG GTA AAG TTC CAG TA GAC
GCT
dye/quencher: Tamra in 5′; Dabcyl in 3′
MG reverse primer (L-MG 1704, 24 nt):
complementary to positions 1681 to 1704 of
SEQ ID NO: 3
SEQ ID NO: 36
ATA CTC CAA TAC CAC CTT AGG CAC

### 1.2.2.4. Primer Pair (U MG 1501/LMG 1704), Amplicon of 203 bp

MG target sequence (203 bp): positions 1501 to
1704 of SEQ ID NO: 3
SEQ ID NO: 38

| | |
|---|---|
| 1501 | gcaaaaatgg aaaacccctc aacggtgcaa aggggtttaa |
| | atggcgagcc tatctttgat |
| 1561 | ccttttaaag gctttggttt aactggtaat gcccctactg |
| | attggaatga gatcaaaggt |
| 1621 | aaagttccag tagaagtagt tcaatccccc cattccccca |
| | acctctattt tgtgttacta |
| 1681 | gtgcctaagg tggcattaga gtat |

In bold and underlined characters, are shown (from 5′ to 3′) the sequences of the MG forward primer (U-MG 1501), of the target for the MG probe (MGBR 178q Tamra/Dabcyl), and of the target for the MG reverse primer (L-MG 1704).

MG forward (U-MG 1501, 25 nt), positions 1501 to
1525 of SEQ ID NO: 3
SEQ ID NO: 39
GCA AAA ATG GAA AAC CCC TCA ACG G
MG probe (MGBR 204u Tamra/Dabcyl, 27 nt),
positions 1600 to 1626 of SEQ ID NO: 3
SEQ ID NO: 40
GAT TGG AAT GAG ATC AAA GGT AAA GTT
beacon ® arms: CGC CCT in 5′; AGG GCG in 3′
probe in beacon ® format:
(SEQ ID NO: 41)
CGC CCT GAT TGG AAT GAG ATC AAA GGT AAA GTT AGG
GCG
dye/quencher: Tamra in 5′; Dabcyl in 3′
MG reverse primer (L-MG 1704, 24 nt):
complementary to positions 1681 to 1704 of
SEQ ID NO: 3
SEQ ID NO: 36
ATA CTC CAA TAC CAC CTT AGG CAC

1.3. Selection of Primers and Probe for Neisseria gonorrhoeae (NG)

The selection of an appropriate nucleotide target is difficult to achieve for NG. The genome of NG is indeed highly homologous to the one of M. meningitidis (at about 98%), and is homologous to the genome of commensal species such as N. cinerea, N. lactamica, N. sicca, N. subflava, N. mucosa.

We selected the pilE gene to detect NG. The pilE gene codes for type IV pili. It is present within every pathogenic NG, and is absent from non-pathogenic NG (NG strains often loose this gene when they are grown in vitro).

The pilE gene is present at a variable copy number, and is often subject to nucleotide variations. The pilE gene is also present in N. lactamica and N. cinerea, and may also be present in some other bacterial geni, such as some Pseudomonas, Bacteroides, and Bacillus.

A primer pair (U-pilE 159/L-pilE 406) has been selected. The obtained amplicon is of 252 bp. A fluorescent (ATTO 647N/Dabcyl) probe has been selected to hybridize to the amplicon.

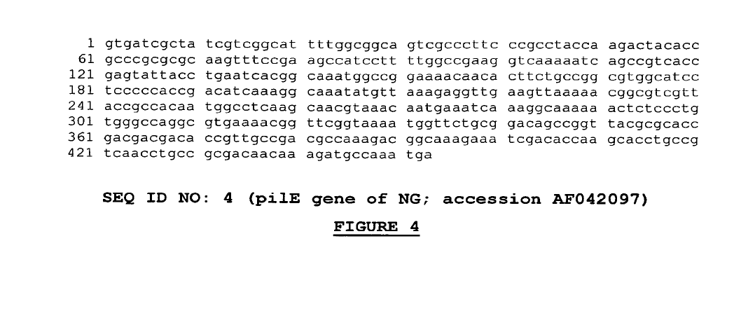

The sequence of the pilE gene, which is accessible under accession number AF042097, is shown on the enclosed FIG. 4 (SEQ ID NO: 4).

| NG sub-sequence selected within pilE of SEQ ID NO: 4 | |
|---|---|
| (positions 101-380): | |
| SEQ ID NO: 42 | |
| 101 | gtcaaaaatc agccgtcacc |
| 121 | gagtattacc tgaatcacgg caaatggccg gaaaacaaca cttctgccgg cgtggcatcc |
| 181 | tcccccaccg acatcaaagg caaatatgtt aaagaggttg aagttaaaaa cggcgtcgtt |
| 241 | accgccacaa tggcctcaag caacgtaaac aatgaaatca aaggcaaaaa actctccctg |
| 301 | tgggccaggc gtgaaaacgg ttcggtaaaa tggttctgcg gacagccggt tacgcgcacc |
| 361 | gacgacgaca ccgttgccga |

Underlined are shown (from 5′ to 3′) the sequences of the NG forward primer (U-pilE 159), of the target for the NG probe (pilEc), and of the target for the NG reverse primer (L-pilE 406).

NG target sequence (252nt): positions 114 to 365 of SEQ ID NO: 4
SEQ ID NO: 43
cgtcacc
gagtattacc tgaatcacgg caaatggccg gaaaacaaca cttctgccgg cgtggcatcc
tcccccaccg acatcaaagg caaatatgtt aaagaggttg aagttaaaaa cggcgtcgtt
accgccacaa tggcctcaag caacgtaaac aatgaaatca aaggcaaaaa actctccctg
tgggccaggc gtgaaaacgg ttcggtaaaa tggttctgcg gacagccggt tacgcgcacc
gacga
NG forward primer (U-pilE 159; 19nt): positions 114 to 132
of SEQ ID NO: 4
SEQ ID NO: 44
CGT CAC CGA GTA TTA CCT G
NG probe (pilEc; 22nt): complementary to positions 285 to 306
de SEQ ID NO: 4
SEQ ID NO: 45
GGC CCA CAG GGA GAG TTT TTT G
beacon ® arms: ACT GCG in 5′; CGC AGT in 3′
Probe in beacon ® format:
(SEQ ID NO: 46)
ACT GCG GGC CCA CAG GGA GAG TTT TTT G CGC AGT
dye/quencher: Atto 647 N in 5′; Dabcyl in 3′
NG reverse primer (L-pilE 406; 17nt): complementary to positions
349 to 365 of SEQ ID NO: 4
SEQ ID NO: 47
TCG TCG GTG CGC GTA AC

### 1.4. Internal Control (IC):

The Internal Control (IC) consists in a single-stranded random sequence.

The IC comprises a sequence which is unrelated to CT, MG and NG, and which preferably is also unrelated to human nucleic acids. The IC further comprises a sequence located in 5′ of this unrelated sequence and a sequence located 3′ of this unrelated sequence, wherein one of said 5′- and 3′-located sequences has the sequence of a primer of a primer pair, and the other of said 5′- and 3′-located sequences is the complementary sequence of the other primer of the same primer pair, such that said primer pair can hybridize to said IC in such locations and following such an orientation that this primer pair can function as an amplification forward and reverse primer pair on said IC. For example, said 5′-located sequence is identical to the forward primer of a primer pair, and said 3′-located sequence is complementary to the reverse primer of said primer pair.

This primer pair may be a primer pair which is used for the detection of CT, MG, or NG, or it can be a different primer pair, which has then to be specifically added in the PCR mix when implementing a multiplex PCR.

In the present example, the IC is of 92 bases, and comprises 5′- and 3′-located sequences which hybridize to a primer pair (primer pair IS 368 and IS 569), which is different from the CT, MG and NG primer pairs.

Internal Control sequence (92nt):
SEQ ID NO: 48
ATTGGATCCCAGCACGCTAATTACCCAGTATCAGATGACGTGGCAGCC
ATGAGAGTGGGACAGTCGTCCTTCAAGGAGCACATCAGCGGATC
Underlined are shown (from 5′ to 3′) the
sequences of the IC forward primer (IS 368), of
the target of the IC probe, and of the target of
the reverse primer (IS 569).
IC forward primer (IS 368; 17nt)
SEQ ID NO: 49
CAGCACGCTAATTACCC
IC probe (SIMB ATTO 590; 23nt)
SEQ ID NO: 50
CCC ACT CTC ATG GCT GCC ACG TC
beacon arms: CAGGCG in 5′; CGCCTG in 3′
probe in beacon format:
(SEQ ID NO: 51)
CAGGCG CCC ACT CTC ATG GCT GCC ACG TC
CGCCTG
dye/quencher: ATTO 590 in 5′; Dabcyl in 3′
IC reverse primer (IS 569; 17nt)
SEQ ID NO: 52
GCTGATGTGCTCCTTGA

1.5. 16S rRNA

A fragment of the 16S rRNA which is present in all micro-organisms is also amplified. The following 16S rRNA primers have been used:

SEQ ID NO: 53

S1-F: AGT TTG ATC ATG GCT CAG

SEQ ID NO: 54

S1-R: GTA TTA CCG CGG CTG CT

### 1.6. Sequences and SEQ ID NO:

TABLE 1
SEQ ID NO sequences

SEQ ID NO:

CT, MG and NG reference sequences

| | | |
|---|---|---|
| 1 | pCHL1 plasmid of CT | Accession number J03321 |
| 2 | 5′ region of adhesin gene of MG | Accession number X91074 |
| 3 | Adhesin gene of MG | Accession number M31431 |
| 4 | pilE gene of NG | Accession number AF042097 |

CT real-time amplification systems of the invention
(CT systems n° 1, n° 2)

| | | |
|---|---|---|
| 5 | Selected CT sub-sequence | 5571-5760 from SEQ ID NO: 1 |
| 6 | CT target (175 nt) | 5580-5754 from SEQ ID NO: 1 |
| 7 | CT forward primer U-PC 5580 | 5580-5599 from SEQ ID NO: 1 |
| 8 | CT probe SCT 175b | 5613-5636 from SEQ ID NO: 1 |
| 9 | CT probe SCT 175b in beacon format | |
| 12 | CT reverse primer L-PC 5754 | 5733-5754 from SEQ ID NO: 1 |
| 5 | Selected CT sub-sequence | 5571-5760 from SEQ ID NO: 1 |
| 6 | CT target (175 nt) | 5580-5754 from SEQ ID NO: 1 |
| 7 | CT forward primer U-PC 5580 | 5580-5599 from SEQ ID NO: 1 |
| 10 | CT probe SCT 175c | 5635-5661 from SEQ ID NO: 1 |
| 11 | CT probe SCT 175c in beacon format | |
| 12 | CT reverse primer L-PC 5754 | 5733-5754 from SEQ ID NO: 1 |

MG real-time amplification systems of the invention
(MG system n° 1, n° 2, n° 3, n° 4, n° 5)

| | | |
|---|---|---|
| 13 | Selected MG sub-sequence | 1-270 from SEQ ID NO: 2 |
| 14 | MG target (258 nt) | 2-259 from SEQ ID NO: 2 |
| 15 | MG forward primer U-MG 1320 | 2-25 from SEQ ID NO: 2 |
| 16 | MG probe SF-MG 258c | 136-162 from SEQ ID NO: 2 |
| 17 | MG probe SF-MG 258c in beacon format | |
| 18 | MG reverse primer L-MG 1578 | 237-259 from SEQ ID NO: 2 |
| 19 | Selected MG sub-sequence | 1140-1290 from SEQ ID NO: 3 |
| 20 | MG target (140 nt) | 1144-1283 from SEQ ID NO: 3 |
| 21 | MG forward primer U-MG 1144 | 1144-1164 from SEQ ID NO: 3 |
| 22 | MG probe MGBR 140c | 1208-1232 from SEQ ID NO: 3 |
| 23 | MG probe MGBR 140c in beacon format | |
| 24 | MG reverse primer L-MG 1283 | 1262-1283 from SEQ ID NO: 3 |
| 25 | Selected MG sub-sequence | 1060-1250 from SEQ ID NO: 3 |
| 26 | MG target (186 nt) | 1064-1249 from SEQ ID NO: 3 |
| 27 | MG forward primer U-MG 1087 | 1064-1087 from SEQ ID NO: 3 |
| 28 | MG probe MGBR 186j | 1142-1168 from SEQ ID NO: 3 |
| 29 | MG probe MGBR 186j in beacon format | |
| 30 | MG reverse primer L-MG 1249 | 1226-1249 from SEQ ID NO: 3 |
| 31 | Selected MG sub-sequence | 1520-1710 from SEQ ID NO: 3 |
| 32 | MG target (178 nt) | 1527-1704 from SEQ ID NO: 3 |
| 33 | MG forward primer U-MG 1527 | 1527-1550 from SEQ ID NO: 3 |
| 34 | MG probe MGBR 178q | 1607-1630 from SEQ ID NO: 3 |
| 35 | MG probe MGBR 178q in beacon format | |
| 36 | MG reverse primer L-MG 1704 | 1681-1704 from SEQ ID NO: 3 |
| 37 | Selected MG sub-sequence | 1500-1710 from SEQ ID NO: 3 |
| 38 | MG target (204 nt) | 1501-1704 from SEQ ID NO: 3 |
| 39 | MG forward primer U-MG 1501 | 1501-1525 from SEQ ID NO: 3 |
| 40 | MG probe MGBR 204u | 1600-1626 from SEQ ID NO: 3 |
| 41 | MG probe in beacon format | |
| 36 | MG reverse primer L-MG 1704 | 1681-1704 from SEQ ID NO: 3 |

NG real-time amplification system of the invention

| | | |
|---|---|---|
| 42 | Selected NG sub-sequence | 101-380 from SEQ ID NO: 4 |
| 43 | NG target (252 nt) | 114-365 from SEQ ID NO: 4 |
| 44 | NG forward primer U-pilE 159 | 114-132 from SEQ ID NO: 4 |
| 45 | NG probe pilEc | 285-306 from SEQ ID NO: 4 |
| 46 | NG probe in beacon format | |
| 47 | NG reverse primer L-pilE 406 | 349-365 from SEQ ID NO: 4 |

Internal Control (IC)

| | | |
|---|---|---|
| 48 | IC sequence (92 nt) | Unrelated to CT, MG and NG |
| 49 | IC forward primer IS 368 | |
| 50 | IC probe | |
| 51 | IC probe in beacon format | |
| 52 | IC reverse primer IS 569 | |

16S rRNA

| | | |
|---|---|---|
| 53 | 16S rRNA forward primer S1-F | Fragment of 16S rRNA |
| 54 | 16S rRNA reverse primer S1-R | Fragment of 16S rRNA |
| 55 | MG primer MgPa-1 | |
| 56 | MG primer MgPa-3 | |
| 57 | MG Southern blot probe MgPa-2 | |

### 2. Example 2

### Specificity and Sensitivity of CT, MG and NG Real-Time Amplification Systems of the Invention

specificity in real-time simplex PCR (CT or MG or NG real-time amplification system on a panel of bacterial strains);
sensitivity of these systems in real-time multiplex PCR (CT+MG+NG+IC systems together in the same mix, on a mix of a pre-determined quantity of CT, MG and NG DNA).

### 2.1. Materials and Methods:

### 2.1.1. Description of the Extraction Method:

Lysis is performed in the presence of detergents and of Chelex™ X-100 beads (i.e., a resin which binds divalent ions, and which also is a chaotropic agent).

### Composition of the Lysis Buffer:

8% of Chelex™ X-100 resin (Bio-Rad, ref.: 142-1253); 0.5% NP-40 (Sigma, ref.: Igepal 1-3021); 0.5% Tween 20 (VWR, ref.: 28829296) in Tris 10 mM buffer (Sigma, ref.: T-6791); EDTA 1 mM (Sigma, ref.: E-1644) pH 8.3.

### Method:

collect 1 mL of sample (sample of urine, or of transport medium), and centrifuge it for 10 minutes at 8,000 rpm; discard the whole supernatant,
add 400 μL lysis buffer in the centrifugation pellet,
add 10 μL of liquid internal control,
vortex 30″,
incubation 10 min at 95° C.,
centrifugation for 5 min at 8,000 rpm,
PCR on 10 μL of supernatant.

The internal control (IC) is added at the extraction step.

The IC solution is of 9.6 10⁴ copies of IC per 10 μL, to obtain 2.4 10³ copies per PCR test PCR after extraction.

The negative control is achieved by collecting 400 μL of lysis buffer into which the IC is added.

### 2.1.2. PCR Conditions:

Reactants:

Hot Start Taq Polymerase Qiagen (5 U/μL, ref 203205), containing the PCR buffer

dNTP: Promega ref U151 (4×25 mM)

MgCl₂: Sigma, ref M-2670

PVP10: Sigma, ref PVP-10

Glycerol: VWR, ref 24388295

Thermocyclor: iQ1 (Bio-Rad)

2.1.3. Amplification of a Fragment of the Gene Coding for 16S rRNA

The products amplified by the 16S rRNA primers (S1-F, and S1-R) are visualised by a Bet staining after electrophoretic migration on a 7.5% acrylamide gel.

2.1.4. Real-Time Simplex PCR for Chlamydia trachomatis

Composition of the Mix

In a 1×PCR mix, add: 0.2 μM of the SCT 175b probe (SEQ ID NO: 9), 0.5 μM of each CT primers (U-PC 5580—SEQ ID NO: 7—, et L-PC 5754—SEQ ID NO: 12—), 5% Glycerol, 0.3% PVP 10, 2 U of Taq Polymerase, 1 mM dNTP and 6 mM final of MgCl₂.

10 μL of DNA are added to this mix.

Thermocycling:

First cycle: 15″ at 95° C.

Second cycle: 30″ at 95° C.

Third cycle: 45″ at 56° C.

Fourth cycle: 30″ at 72° C.

Repeat 50 times from cycle 2 to cycle 4.

2.1.5. Real-Time Simplex PCR for Mycoplasma genitalium

Composition of the Mix

In a 1×PCR mix, add: 0.2 μM of the MG probe (SF-MG 258c—SEQ ID NO: 17—), 0.5 μM of each MG primers (U-MG 1320—SEQ ID NO: 15—, and L-MG 1578—SEQ ID NO: 18—), 5% Glycerol, 0.3% PVP 10, 2 U of Taq Polymerase, 1 mM dNTP and 6 mM final of MgCl₂.

10 μL of DNA are added to this mix.

Thermocycling:

First cycle: 15″ at 95° C.

Second cycle: 30″ at 95° C.

Third cycle: 45″ at 57° C.

Fourth cycle: 30″ at 72° C.

Repeat 50 times from cycle 2 to cycle 4

2.1.6. Real-Time Simplex PCR for Neisseria gonorrhoeae

Composition of the Mix

In a 1×PCR mix, add: 0.2 μM of the NG probe (pilEc—SEQ ID NO: 46—), 0.5 μM of each NG primers (U pilE 159—SEQ ID NO: 44—, et L-pilE 406—SEQ ID NO: 47—), 5% Glycerol, 0.3% PVP 10, 2 U of Taq Polymerase, 1 mM dNTP and 4 mM final of MgCl₂.

10 μL of DNA are added to this mix.

Thermocycling:

First cycle: 15″ at 95° C.

Second cycle: 30″ at 95° C.

Third cycle: 45″ at 57° C.

Fourth cycle: 30″ at 72° C.

Repeat 50 times from cycle 2 to cycle 4

### 2.1.7. Real-Time Multiplex PCR

Composition of the Mix

In a 1×PCR mix, add the following primers:

CT forward primer U-PC 5580 (SEQ ID NO: 7) at 0.125 μM,

CT reverse primer L-PC 5754 (SEQ ID NO: 12) at 0.5 μM,

MG forward primer U-MG 1320 (SEQ ID NO: 15) at 0.025 μM,

MG reverse primer L-MG 1578 (SEQ ID NO: 18) at 1.25 μM,

NG forward primer U-pilE 159 (SEQ ID NO: 44) at 0.85 μM,

NG reverse primer L-pilE 406 (SEQ ID NO: 47) at 0.25 μM,

IC forward primer IS 368 (SEQ ID NO: 49) at 0.5 μM, and

IC reverse primer IS 569 (SEQ ID NO: 52) at 0.5 μM.

The probes are used at the following concentrations:

CT probe SCT 175b (SEQ ID NO: 9) at 0.15 μM (fluorophore FAM),

MG probe SF-MG 258c (SEQ ID NO: 17) at 0.2 μM (fluorophore TAMRA), and

NG probe pilEc (SEQ ID NO: 46) at 0.2 μM (fluorophore ATTO 647N).

IC probe SIMB (SEQ ID NO: 51) at 0.15 μM (fluorophore ATTO-590),

The following reactants are further added:

5% Glycerol, 0.3% PVP 10, 2 U of Taq Polymerase, 1 mM dNTP and 5 mM final of

MgCl₂.

10 μL of DNA are added to this mix.

Thermocycling:

First cycle: 15″ at 95° C.

Second cycle: 30″ at 95° C.

Third cycle: 50″ at 58° C.

Fourth cycle: 30″ at 72° C.

Repeat 50 times from cycle 2 to cycle 4

Starting Material:

CT DNA: Chlamydia trachomatis LGV II strain 434, available from ABi, lot 141-115, 1.63 10¹⁰ elementary body/ml, 100 μL at 50 ng/μL.

ABi is Advanced Biotechnologies Inc., RiversPark II, 9108 Guilford Road, Columbia, Md. 21046-2701, U.S.A.

MG DNA: strain G-37 available from ATCC, deposit number 33530 (source culture=ATCC 33530D), lot 2305272, concentration 200 ng/μL.

ATCC is: American Type Culture Collection, 10801 University Boulevard Manassas, Va. 20110-2209, U.S.A.

NG DNA: strain 107031 from the CNCM (Collection de l'Institut Pasteur, BP 52, 25 rue du Docteur Roux, 75724 Paris cedex 15, France), reference strain for antimicrobial disk susceptibility test (count has been made on Petri dish).

Quantity of IC: 1,000 copies per PCR

### 2.2. Results:

The CT and MG primers and probes have a perfect specificity: they do not cross-react with any nucleic acid other than CT or MG nucleic acids (respectively); they notably do not cross-react with other bacterial or with human nucleic acids.

The NG primers and probes are specific for NG, except that they cross-react with N. meningitidis.

### 2.2.1. Specificity Test for the CT System in Real-Time Simplex PCR:

The amplicon is of 175 bp.

The CT primers and probes of the invention detect all CT serovars. They notably detect the following serovars: A, B, Ba, C, D, E, F, H, I, J, K, L1, L2a and L3.

The CT primers and probes of the invention have also been tested on 24 different bacterial strains which may be found in the urogenital sphere, and were shown to be non cross-reactive. For these 24 other strains, presence of DNA was confirmed by end-point PCR using the 16S rRNA primers (S1R and S1F primers), followed by deposition on gel. Specificity results are shown in table 1 below (column “simplex CT”).

### 2.2.2. Specificity Test for the MG System in Real-Time Simplex PCR:

The amplicon is of 258 bp.

The MG primers and probes of the invention detect the nine MG strains that have been tested (strains G-37, 2282, 2288, 2300, 2321, 2341, M30, UTMB1 and TW-10-51).

The MG primers and probes of the invention did not cross-react with any of the 29 non-MG bacterial DNA tested.

Results are reported in the above table 2, column “simplex MG”.

TABLE 2
specificity of the CT et MG systems

| | Simplex | Simplex | 16S |
|---|---|---|---|
| Strain | CT | MG | rRNA |
| human DNA | negative | negative | // |
| Chlamydia trachomatis, serovar A, clinical strain, CHU Bordeaux | positive | NT | positive |
| Chlamydia trachomatis, serovar B, clinical strain, CHU Bordeaux | positive | NT | positive |
| Chlamydia trachomatis, serovar Ba, clinical strain, CHU Bordeaux | positive | NT | positive |
| Chlamydia trachomatis, serovar C, clinical strain, CHU Bordeaux | positive | NT | positive |
| Chlamydia trachomatis, serovar D, strain UW-3/Cx, ATCC VR 885 | positive | NT | positive |
| Chlamydia trachomatis, serovar F, clinical strain, CHU Bordeaux | positive | NT | positive |
| Chlamydia trachomatis, serovar H, clinical strain, CHU Bordeaux | positive | NT | positive |
| Chlamydia trachomatis, serovar I, clinical strain, CHU Bordeaux | positive | NT | positive |
| Chlamydia trachomatis, serovar J, clinical strain, CHU Bordeaux | positive | NT | positive |
| Chlamydia trachomatis, serovar K, clinical strain, CHU Bordeaux | positive | NT | positive |
| Chlamydia trachomatis, serovar L1, clinical strain, CHU Bordeaux | positive | NT | positive |
| Chlamydia trachomatis, serovar L2a, clinical strain, CHU Bordeaux | positive | NT | positive |
| Chlamydia trachomatis, serovar L3, clinical strain, CHU Bordeaux | positive | NT | positive |
| Escherichia coli ATCC 25922 | negative | negative | positive |
| Enterobacter cloacae ATCC 13047 | negative | negative | positive |
| Staphylococcus saprophyticus, clinical strain | negative | negative | positive |
| Enterococcus faecium, clinical strain | negative | negative | positive |
| Enterococcus faecalis, clinical strain | negative | negative | positive |
| Proteus mirabilis ATCC 29906 | negative | negative | positive |
| Lactobacillus acidophilus, clinical strain | negative | negative | positive |
| Bacillus subtilis, clinical strain | negative | NT | positive |
| Gardnerella vaginalis, clinical strain | negative | negative | positive |
| Neisseria gonorrhoeae, 5 clinical strains | negative | negative | positive |
| Neisseria cinerea DSMZ 4630 | NT | negative | positive |
| Neisseria lactamica DSMZ 4691 | NT | negative | positive |
| Neisseria sicca, clinical strain | NT | negative | positive |
| Neisseria meningitidi, clinical strain | NT | negative | positive |
| Staphylococcus aureus ATCC 25923 | negative | negative | positive |
| Staphylococcus simulans strain Institut Pasteur | NT | negative | positive |
| Pseudomonas aeruginosa, ATCC 27853 | negative | negative | positive |
| Klebsiella pneumoniae, CIP 104298 | negative | NT | positive |
| Strepcococcus agalactiae, ATCC 12403 | negative | negative | positive |
| Streptococcus bovis CIP 105065 | negative | negative | positive |
| Acinetobacter baumanii, ATCC 49139 | negative | negative | positive |
| Staphylococcus epidermidis ATCC 49139 | negative | negative | positive |
| Candida albicans clinical strain | negative | negative | positive |
| Mycoplasma pneumoniae clinical strain | negative | negative | positive |
| Neisseria mucosa clinical strain | negative | negative | positive |
| Rhodococcus equi clinical strain | negative | negative | positive |
| Moraxella catarrhalis, clinical strain | negative | negative | positive |
| Mycoplasma genitalium, strain G-37, ATCC 33530 | negative | positive | positive |
| Mycoplasma genitalium, strain 2282, clinical strain, | NT | positive | positive |
| Statens Serum Institut, Danemark | | | |
| Mycoplasma genitalium, strain 2288, clinical strain, | NT | positive | positive |
| Statens Serum Institut, Danemark | | | |
| Mycoplasma genitalium, strain 2300, clinical strain, | NT | positive | positive |
| Statens Serum Institut, Danemark | | | |
| Mycoplasma genitalium, strain 2321, clinical strain, | NT | positive | positive |
| Statens Serum Institut, Danemark | | | |
| Mycoplasma genitalium, strain 2341, clinical strain, | NT | positive | positive |
| Statens Serum Institut, Danemark | | | |
| Mycoplasma genitalium, strain M30, clinical strain, | NT | positive | positive |
| CHU Bordeaux | | | |
| Mycoplasma genitalium, strain UTMB1, clinical strain, | NT | positive | positive |
| CHU Bordeaux | | | |
| Mycoplasma genitalium, strain TW-10-51, clinical strain, | NT | positive | positive |
| CHU Bordeaux | | | |
| Mycoplasma orale ATCC 23714 | negative | negative | positive |
| Mycoplasma fermentans strain PG18, clinical strain, | NT | negative | positive |
| CHU Bordeaux | | | |
| Mycoplasma penetrans strain GTU64, clinical strain, | NT | negative | positive |
| CHU Bordeaux | | | |

NT: not tested
CHU Bordeaux = Hospital of Bordeaux, France

### 2.2.3. Specificity Test for the NG System in Real-Time Simplex PCR:

The amplicon is of 252 bp.

The NG primers and probes of the invention have been assayed in real-time PCR on 55 different NG strains. All results were positive.

The NG primers and probes of the invention have also been tested on several Neisseria species other than NG. Results are shown in table 3 below. No amplification was obtained with the following species: N. sicca (3 strains), N. polysaccharia (1 strain), N. subflava (4 strains), N. mucosa (3 strains), N. cinerea (1 strain), and N. lactamica (2 strains).

Among the 16 N. meningitidis strains that have been tested, 10 gave a positive response (NM cross-reaction).

TABLE 3
specificity of the NG system in simplex
PCR with different Neisseria species

| Strain | Simplex NG | 16S rRNA |
|---|---|---|
| Human DNA | negative | positive |
| N. cinerea (1850) DSMZ 4630 | negative | positive |
| N. lactamica (1851) DSMZ 4691 | negative | positive |
| N. lactamica (1874) | negative | positive |
| N. meningitidis 10 clinical strains | positive | positive |
| N. meningitidis 6 clinical strains | negative | positive |
| N. mucosa (1853, 1870, 1871) | negative | positive |
| N. polysaccharia (1852) CIP 100113T | negative | positive |
| N. sicca (1854, 1872, 1873) | negative | positive |
| N. subflava (1855, 1876, 1877) | negative | positive |
| N. subflava (1875) CIP 73.13 | negative | positive |
| N. gonorrhoeae, 55 clinical strains | positive | positive |

The specificity of the NG primers of the invention has been assayed in end-point PCR, followed by deposition on gel, with 13 bacterial strains that do not belong to the Neisseria genus. No amplification has been detected. Results are reported in table 4 below.

TABLE 4
specificity relative to non-Neisseria strains

| | End-point PCR | |
|---|---|---|
| Strain | for NG | 16S rRNA |
| Human DNA | negative | // |
| Escherichia coli ATCC 25922 | negative | positive |
| Enterobacter cloacae ATCC 13047 | negative | positive |
| Staphylococcus saprophyticus, clinical strain | negative | positive |
| Enterococcus faecium, clinical strain | negative | positive |
| Enterococcus faecalis, clinical strain | negative | positive |
| Proteus mirabilis ATCC 29906 | negative | positive |
| Bacillus subtilis, clinical strain | negative | positive |
| Garnerella vaginalis, clinical strain | negative | positive |
| Pseudomonas aeruginosa, ATCC 27853 | negative | positive |
| Strepcococcus agalactiae, ATCC 12403 | negative | positive |
| Acinetobacter baumanii, ATCC 49139 | negative | positive |
| Staphylococcus epidermidis ATCC 49139 | negative | positive |
| Moraxella catarrhalis, clinical strain | negative | positive |

### 2.2.4. Results in Multiplex PCR:

PCR Sensitivity

The CT, MG, NG and IC primers and probes of the invention have been assayed in quadruplex on a mix of a pre-determined quantity of CT, MG and NG DNA (the experiment was made in duplicate).

Results are as follows:

TABLE 5
CT probe (FAM)

quadruplex mix

| Copy number | Ct | RFU |
|---|---|---|
| 1000 | 31.75 +/− 0.10 | 400 |
| 100 | 35.20 +/− 0.63 | 300 |
| 10 | 41.30 +/− 2.62 | 100 |
| 1 | ND | |

ND: not detected [symbol]
sensitivity is of 10 copy per PCR

TABLE 6
MG probe (TAMRA)

quadruplex mix

| Copy number | Ct | RFU |
|---|---|---|
| 1000 | 36.93 +/− 2.07 | 140 |
| 100 | 40.63 +/− 0.85 | 100 |
| 10 | ND | |
| 1 | ND | |

ND: not detected [symbol]
sensitivity is of 100 copies per PCR.

TABLE 7
NG probe (ATTO 647N)

Quadruplex mix

| Copy number | Ct | RFU |
|---|---|---|
| 1000 | 27.30 +/− 0.28 | 350 |
| 100 | 31.33 +/− 0.43 | 325 |
| 10 | 34.4 +/− 0.37 | 250 |
| 1 | 37.95 +/− 0.51 | 200 |

[symbol]

Sensitivity is of 1 copy per PCR

TABLE 8
IC probe (ATTO- 590)

Quadruplex mix

| Copy number | Ct | RFU |
|---|---|---|
| 1000 | 34.35 +/− 0.24 | 225 |
| 100 | 34.15 +/− 0.37 | |
| 10 | 34.53 +/− 0.17 | |
| 1 | 34.65 +/− 0.40 | |

[symbol]

There is no Ct variation for the IC, whatever quantity of DNA is being used.

### 3. Example 3

### Specific and Sensitivity of CT, MG and NG Real-Time Amplification Systems of the Invention

specificity in real-time multiplex PCR (CT+MG+NG+IC real-time amplification systems of the invention, together in multiplex in the same mix, tested on a panel of bacterial strains);
sensitivity in real-time multiplex PCR (CT+MG+NG+IC systems together in multiplex in the same mix, tested on a mix of a pre-determined quantity of CT, MG and NG DNA).

### 3.1. Material and Methods:

### Real Time Multiplex PCR

Composition of the First Mix

In a 1×PCR mix, add the following primers:

CT forward primer U-PC 5580 (SEQ ID NO: 7) at 0.125 μM,

CT reverse primer L-PC 5754 (SEQ ID NO: 12) at 0.5 μM,

MG forward primer U-MG 1144 (SEQ ID NO: 21) at 0.5 μM,

MG reverse primer L-MG 1283 (SEQ ID NO: 24) at 0.5 μM,

NG forward primer U-pilE 159 (SEQ ID NO: 44) at 0.85 μM,

NG reverse primer L-pilE 406 (SEQ ID NO: 47) at 0.25 μM,

IC forward primer IS 368 (SEQ ID NO: 49) at 0.5 μM, and

IC reverse primer IS 569 (SEQ ID NO: 52) at 0.5 μM.

The probes are used at the following concentrations:

CT probe SCT 175b (SEQ ID NO: 9) at 0.15 μM (fluorophore FAM),

MG probe MGBR 140c (SEQ ID NO: 23) at 0.2 μM (fluorophore TAMRA

NG probe pilEc (SEQ ID NO: 46) at 0.2 μM (fluorophore ATTO 647N) and

IC probe SIMB (SEQ ID NO: 51) at 0.15 μM (fluorophore ATTO-590),

Composition of the Second Mix

In a 1×PCR mix, add the following primers:

CT forward primer U-PC 5580 (SEQ ID NO: 7) at 0.125 μM,

CT reverse primer L-PC 5754 (SEQ ID NO: 12) at 0.5 μM,

MG forward primer U-MG 1087 (SEQ ID NO: 27) at 0.5 μM,

MG reverse primer L-MG 1249 (SEQ ID NO: 30) at 0.5 μM,

NG forward primer U-pilE 159 (SEQ ID NO: 44) at 0.85 μM,

NG reverse primer L-pilE 406 (SEQ ID NO: 47) at 0.25 μM,

IC forward primer IS 368 (SEQ ID NO: 49) at 0.5 μM, and

IC reverse primer IS 569 (SEQ ID NO: 52) at 0.5 μM.

The probes are used at the following concentrations:

CT probe SCT 175b (SEQ ID NO: 9) at 0.15 μM (fluorophore FAM),

MG probe MGBR 186j (SEQ ID NO: 29) at 0.2 μM (fluorophore TAMRA)

NG probe pilEc (SEQ ID NO: 46) at 0.2 μM (fluorophore ATTO 647N) and

IC probe SIMB (SEQ ID NO: 51) at 0.15 μM (fluorophore ATTO-590),

Composition of the Third Mix

In a 1×PCR mix, add the following primers:

CT forward primer U-PC 5580 (SEQ ID NO: 7) at 0.125 μM,

CT reverse primer L-PC 5754 (SEQ ID NO: 12) at 0.5 μM,

MG forward primer U-MG 1527 (SEQ ID NO: 33) at 0.5 μM,

MG reverse primer L-MG 1704 (SEQ ID NO: 36) at 0.5 μM,

NG forward primer U-pilE 159 (SEQ ID NO: 44) at 0.85 μM,

NG reverse primer L-pilE 406 (SEQ ID NO: 47) at 0.25 μM,

IC forward primer IS 368 (SEQ ID NO: 49) at 0.5 μM, and

IC reverse primer IS 569 (SEQ ID NO: 52) at 0.5 μM.

The probes are used at the following concentrations:

CT probe SCT 175b (SEQ ID NO: 9) at 0.15 μM (fluorophore FAM),

MG probe MGBR 178q (SEQ ID NO: 35) at 0.2 μM (fluorophore TAMRA

NG probe pilEc (SEQ ID NO: 46) at 0.2 μM (fluorophore ATTO 647N) and

IC probe SIMB (SEQ ID NO: 51) at 0.15 μM (fluorophore ATTO-590),

Composition of the Fourth Mix

In a 1×PCR mix, add the following primers:

CT forward primer U-PC 5580 (SEQ ID NO: 7) at 0.125 μM,

CT reverse primer L-PC 5754 (SEQ ID NO: 12) at 0.5 μM,

MG forward primer U-MG 1501 (SEQ ID NO: 39) at 0.5 μM,

MG reverse primer L-MG 1704 (SEQ ID NO: 36) at 0.5 μM,

NG forward primer U-pilE 159 (SEQ ID NO: 44) at 0.85 μM,

NG reverse primer L-pilE 406 (SEQ ID NO: 47) at 0.25 μM,

IC forward primer IS 368 (SEQ ID NO: 49) at 0.5 μM, and

IC reverse primer IS 569 (SEQ ID NO: 52) at 0.5 μM.

The probes are used at the following concentrations:

CT probe SCT 175b (SEQ ID NO: 9) at 0.15 μM (fluorophore FAM),

MG probe MGBR 204u (SEQ ID NO: 41) at 0.2 μM (fluorophore TAMRA)

NG probe pilEc (SEQ ID NO: 46) at 0.2 μM (fluorophore ATTO 647N) and

IC probe SIMB (SEQ ID NO: 51) at 0.15 μM (fluorophore ATTO-590),

The following reactants are further added:

5% Glycerol, 0.3% PVP 10, 2 U of Taq Polymerase, 1 mM dNTP and 5 mM final of MgCl₂.

10 μL of DNA are added to this mix.

Thermocycling:

First cycle: 15″ at 95° C.

Second cycle: 30″ at 95° C.

Third cycle: 50″ at 58° C.

Fourth cycle: 30″ at 72° C.

Repeat 50 times from cycle 2 to cycle 4

16S rRNA PCR

cf. chapter 1.6.1

### 3.2. Results

### 3.2.1. Specificity Test for the MG Systems in Real-Time Multiplex PCR

The MG primers and probes of the invention detect the nine MG strains that have been tested (strains G-37, 2282, 2288, 2300, 2321, 2341, M30, UTMB1 and TW-10-51).

The MG primers and probes of the invention have also been tested on 27 different bacterial strains which may be found in the urogenital sphere, and were shown to be non cross-reactive. For these 27 different bacterial strains, presence of DNA was confirmed by end-point PCR using the 16S rRNA primers (S1R and S1F primers), followed by deposition on gel.

Results are reported in the above tables.

TABLE 9
specificity of the MG system in real-time multiplex PCR

| | First | Second | Third | Fourth | 16S |
|---|---|---|---|---|---|
| | Multiplex | multiplex | multiplex | multiplex | rRNA |
| | Amplicon size | | | | |
| Strain | 140 nt | 186 nt | 178 nt | 204 nt | |
| human DNA | negative | negative | negative | negative | // |
| Chlamydia trachomatis, serovar D, strain | negative | negative | negative | negative | positive |
| UW-3/Cx, ATCC VR 885 | | | | | |
| Chlamydia trachomatis, serovar L1, clinical | negative | negative | negative | negative | positive |
| strain, CHU Bordeaux | | | | | |
| Escherichia coli ATCC 25922 | negative | negative | negative | negative | positive |
| Enterobacter cloacae ATCC 13047 | negative | negative | negative | negative | positive |
| Staphylococcus saprophyticus, clinical | negative | negative | negative | negative | positive |
| strain | | | | | |
| Enterococcus faecium, clinical strain | negative | negative | negative | negative | positive |
| Enterococcus faecalis, clinical strain | negative | negative | negative | negative | positive |
| Proteus mirabilis ATCC 29906 | negative | negative | negative | negative | positive |
| Lactobacillus acidophilus, clinical strain | negative | negative | negative | negative | positive |
| Bacillus subtilis, clinical strain | negative | negative | negative | negative | positive |
| Gardnerella vaginalis, clinical strain | negative | negative | negative | negative | positive |
| Neisseria gonorrhoeae, 1 clinical strain | negative | negative | negative | negative | positive |
| Staphylococcus aureus ATCC 25923 | negative | negative | negative | negative | positive |
| Pseudomonas aeruginosa, ATCC 27853 | negative | negative | negative | negative | positive |
| Klebsiella pneumoniae, CIP 104298 | negative | negative | negative | negative | positive |
| Strepcococcus agalactiae, ATCC 12403 | negative | negative | negative | negative | positive |
| Streptococcus bovis CIP 105065 | negative | negative | negative | negative | positive |
| Acinetobacter baumanii, ATCC 49139 | negative | negative | negative | negative | positive |
| Staphylococcus epidermidis ATCC 49139 | negative | negative | negative | negative | positive |
| Candida albicans clinical strain | negative | negative | negative | negative | positive |
| Mycoplasma pneumoniae clinical strain | negative | negative | negative | negative | positive |
| Neisseria mucosa clinical strain | negative | negative | negative | negative | positive |
| Rhodococcus equi clinical strain | negative | negative | negative | negative | positive |
| Moraxella catarrhalis, clinical strain | negative | negative | negative | negative | positive |
| Mycoplasma genitalium, strain G-37, | positive | positive | positive | positive | positive |
| ATCC 33530 | | | | | |
| Mycoplasma genitalium, strain 2282, | positive | positive | positive | positive | positive |
| clinical strain, Statens Serum Institut, Danemark | | | | | |
| Mycoplasma genitalium, strain 2288, | positive | positive | positive | positive | positive |
| clinical strain, Statens Serum Institut, Danemark | | | | | |
| Mycoplasma genitalium, strain 2300, | positive | positive | positive | positive | positive |
| clinical strain, Statens Serum Institut, Danemark | | | | | |
| Mycoplasma genitalium, strain 2321, | positive | positive | positive | positive | positive |
| clinical strain, Statens Serum Institut, Danemark | | | | | |
| Mycoplasma genitalium, strain 2341, | positive | positive | positive | positive | positive |
| clinical strain, Statens Serum Institut, Danemark | | | | | |
| Mycoplasma genitalium, strain M30, | positive | positive | positive | positive | positive |
| clinical strain, CHU Bordeaux | | | | | |
| Mycoplasma genitalium, strain UTMB1, | positive | positive | positive | positive | positive |
| clinical strain, CHU Bordeaux | | | | | |
| Mycoplasma genitalium, strain TW-10-51, | Positive | positive | Positive | positive | positive |
| clinical strain, CHU Bordeaux | | | | | |
| Mycoplasma orale ATCC 23714 | negative | negative | negative | negative | positive |
| Mycoplasma fermentans strain PG18, | negative | negative | negative | negative | positive |
| clinical strain, CHU Bordeaux | | | | | |
| Mycoplasma penetrans strain GTU64, | negative | negative | negative | negative | positive |
| clinical strain, CHU Bordeaux | | | | | |

NT: not tested
CHU Bordeaux = Hospital of Bordeaux, France

TABLE 10
specificity of the CT system in real-time multiplex PCR

| | First | Second | Third | Fourth | 16S |
|---|---|---|---|---|---|
| | Multiplex | multiplex | multiplex | multiplex | rRNA |
| | Amplicon size | | | | |
| Strain | 175 nt | 175 nt | 175 nt | 175 nt | |
| human DNA | negative | negative | negative | negative | // |
| Chlamydia trachomatis, serovar D, | positive | positive | positive | positive | positive |
| strain UW-3/Cx, ATCC VR 885 | | | | | |
| Chlamydia trachomatis, serovar L1, | positive | positive | positive | positive | positive |
| clinical strain, CHU Bordeaux | | | | | |
| Escherichia coli ATCC 25922 | negative | negative | negative | negative | positive |
| Enterobacter cloacae ATCC 13047 | negative | negative | negative | negative | positive |
| Staphylococcus saprophyticus, clinical | negative | negative | negative | negative | positive |
| strain | | | | | |
| Enterococcus faecium, clinical strain | negative | negative | negative | negative | positive |
| Enterococcus faecalis, clinical strain | negative | negative | negative | negative | positive |
| Proteus mirabilis ATCC 29906 | negative | negative | negative | negative | positive |
| Lactobacillus acidophilus, clinical strain | negative | negative | negative | negative | positive |
| Bacillus subtilis, clinical strain | negative | negative | negative | negative | positive |
| Gardnerella vaginalis, clinical strain | negative | negative | negative | negative | positive |
| Neisseria gonorrhoeae, 1 clinical strain | negative | negative | negative | negative | positive |
| Staphylococcus aureus ATCC 25923 | negative | negative | negative | negative | positive |
| Pseudomonas aeruginosa, ATCC 27853 | negative | negative | negative | negative | positive |
| Klebsiella pneumoniae, CIP 104298 | negative | negative | negative | negative | positive |
| Strepcococcus agalactiae, ATCC 12403 | negative | negative | negative | negative | positive |
| Streptococcus bovis CIP 105065 | negative | negative | negative | negative | positive |
| Acinetobacter baumanii, ATCC 49139 | negative | negative | negative | negative | positive |
| Staphylococcus epidermidis ATCC 49139 | negative | negative | negative | negative | positive |
| Candida albicans clinical strain | negative | negative | negative | negative | positive |
| Mycoplasma pneumoniae clinical strain | negative | negative | negative | negative | positive |
| Neisseria mucosa clinical strain | negative | negative | negative | negative | positive |
| Rhodococcus equi clinical strain | negative | negative | negative | negative | positive |
| Moraxella catarrhalis, clinical strain | negative | negative | negative | negative | positive |
| Mycoplasma genitalium, strain G-37, | negative | negative | negative | negative | positive |
| ATCC 33530 | | | | | |
| Mycoplasma genitalium, strain 2282, | negative | negative | negative | negative | positive |
| clinical strain, Statens Serum Institut, Danemark | | | | | |
| Mycoplasma genitalium, strain 2288, | negative | negative | negative | negative | positive |
| clinical strain, Statens Serum Institut, Danemark | | | | | |
| Mycoplasma genitalium, strain 2300, | negative | negative | negative | negative | positive |
| clinical strain, Statens Serum Institut, Danemark | | | | | |
| Mycoplasma genitalium, strain 2321, | negative | negative | negative | negative | positive |
| clinical strain, Statens Serum Institut, Danemark | | | | | |
| Mycoplasma genitalium, strain 2341, | negative | negative | negative | negative | positive |
| clinical strain, Statens Serum Institut, Danemark | | | | | |
| Mycoplasma genitalium, strain M30, | negative | negative | negative | negative | positive |
| clinical strain, CHU Bordeaux | | | | | |
| Mycoplasma genitalium, strain UTMB1, | negative | negative | negative | negative | positive |
| clinical strain, CHU Bordeaux | | | | | |
| Mycoplasma genitalium, strain TW-10-51, | negative | negative | negative | negative | positive |
| clinical strain, CHU Bordeaux | | | | | |
| Mycoplasma orale ATCC 23714 | negative | negative | negative | negative | positive |
| Mycoplasma fermentans strain PG18, | negative | negative | negative | negative | positive |
| clinical strain, CHU Bordeaux | | | | | |
| Mycoplasma penetrans strain GTU64, | negative | negative | negative | negative | positive |
| clinical strain, CHU Bordeaux | | | | | |

NT: not tested
CHU Bordeaux = Hospital of Bordeaux, France

TABLE 11
specificity of the NG system in real-time multiplex PCR

| | First | Second | Third | Fourth | 16S |
|---|---|---|---|---|---|
| | Multiplex | multiplex | multiplex | multiplex | rRNA |
| | Amplicon size | | | | |
| Strain | 252 nt | 252 nt | 252 nt | 252 nt | |
| human DNA | negative | negative | negative | negative | // |
| Chlamydia trachomatis, serovar D, | negative | negative | negative | negative | positive |
| strain UW-3/Cx, ATCC VR 885 | | | | | |
| Chlamydia trachomatis, serovar L1, | negative | negative | negative | negative | positive |
| clinical strain, CHU Bordeaux | | | | | |
| Escherichia coli ATCC 25922 | negative | negative | negative | negative | positive |
| Enterobacter cloacae ATCC 13047 | negative | negative | negative | negative | positive |
| Staphylococcus saprophyticus, clinical | negative | negative | negative | negative | positive |
| strain | | | | | |
| Enterococcus faecium, clinical strain | negative | negative | negative | negative | positive |
| Enterococcus faecalis, clinical strain | negative | negative | negative | negative | positive |
| Proteus mirabilis ATCC 29906 | negative | negative | negative | negative | positive |
| Lactobacillus acidophilus, clinical strain | negative | negative | negative | negative | positive |
| Bacillus subtilis, clinical strain | negative | negative | negative | negative | positive |
| Gardnerella vaginalis, clinical strain | negative | negative | negative | negative | positive |
| Neisseria gonorrhoeae, 1 clinical strain | positive | positive | positive | positive | positive |
| Staphylococcus aureus ATCC 25923 | negative | negative | negative | negative | positive |
| Pseudomonas aeruginosa, ATCC 27853 | negative | negative | negative | negative | positive |
| Klebsiella pneumoniae, CIP 104298 | negative | negative | negative | negative | positive |
| Strepcococcus agalactiae, ATCC 12403 | negative | negative | negative | negative | positive |
| Streptococcus bovis CIP 105065 | negative | negative | negative | negative | positive |
| Acinetobacter baumanii, ATCC 49139 | negative | negative | negative | negative | positive |
| Staphylococcus epidermidis ATCC 49139 | negative | negative | negative | negative | positive |
| Candida albicans clinical strain | negative | negative | negative | negative | positive |
| Mycoplasma pneumoniae clinical strain | negative | negative | negative | negative | positive |
| Neisseria mucosa clinical strain | negative | negative | negative | negative | positive |
| Rhodococcus equi clinical strain | negative | negative | negative | negative | positive |
| Moraxella catarrhalis, clinical strain | negative | negative | negative | negative | positive |
| Mycoplasma genitalium, strain G-37, | negative | negative | negative | negative | positive |
| ATCC 33530 | | | | | |
| Mycoplasma genitalium, strain 2282, | negative | negative | negative | negative | positive |
| clinical strain, Statens Serum Institut, Danemark | | | | | |
| Mycoplasma genitalium, strain 2288, | negative | negative | negative | negative | positive |
| clinical strain, Statens Serum Institut, Danemark | | | | | |
| Mycoplasma genitalium, strain 2300, | negative | negative | negative | negative | positive |
| clinical strain, Statens Serum Institut, Danemark | | | | | |
| Mycoplasma genitalium, strain 2321, | negative | negative | negative | negative | positive |
| clinical strain, Statens Serum Institut, Danemark | | | | | |
| Mycoplasma genitalium, strain 2341, | negative | negative | negative | negative | positive |
| clinical strain, Statens Serum Institut, Danemark | | | | | |
| Mycoplasma genitalium, strain M30, | negative | negative | negative | negative | positive |
| clinical strain, CHU Bordeaux | | | | | |
| Mycoplasma genitalium, strain UTMB1, | negative | negative | negative | negative | positive |
| clinical strain, CHU Bordeaux | | | | | |
| Mycoplasma genitalium, strain TW-10-51, | negative | negative | negative | negative | positive |
| clinical strain, CHU Bordeaux | | | | | |
| Mycoplasma orale ATCC 23714 | negative | negative | negative | negative | positive |
| Mycoplasma fermentans strain PG18, | negative | negative | negative | negative | positive |
| clinical strain, CHU Bordeaux | | | | | |
| Mycoplasma penetrans strain GTU64, | negative | negative | negative | negative | positive |
| clinical strain, CHU Bordeaux | | | | | |

NT: not tested
CHU Bordeaux = Hospital of Bordeaux, France

### 3.2.2. Results in Quadruplex Mix PCR: Sensitivity

The CT, MG, NG and IC primers and probes of the invention have been assayed in quadruplex on a mix of a pre-determined quantity of CT, MG and NG DNA (the experiment was made in quadricate). Four multiplex are tested, each multiplex containing one MG simplex described on top.

Results are as follows:

TABLE 12
MG probe (TAMRA)

| | First | Second | Third | Fourth |
|---|---|---|---|---|
| | quadruplex mix | quadruplex mix | quadruplex mix | quadruplex mix |
| Copy | Probe MGBR140c | Probe MGBR 186j | Probe MGBR 178q | Probe MGBR 204u |
| number | Ct | Ct | Ct | Ct |
| 1000 | 33.48 +/− 2.03 | 34.58 +/− 0.75 | 37.68 +/− 1.11 | 37.78 +/− 0.64 |
| 100 | 39.75 +/− 2.92 | 40.05 +/− 2.01 | 42.23 +/− 3.81 | 44.20 +/− 2.39 |
| 10 | 41.73 +/− 0.46 | ND | ND | ND |
| 1 | ND | ND | ND | ND |

ND: not detected [symbol]
sensitivity is of 10 copies per PCR for the first quadruplex mix (Probe MGBR140c and 100 copies per PCR for other multiplex

TABLE 13
CT probe (FAM)

| | First | Second | Third | Fourth |
|---|---|---|---|---|
| | quadruplex mix | quadruplex mix | quadruplex mix | quadruplex mix |
| Copy | Probe MGBR140c | Probe MGBR 186j | Probe MGBR 178q | Probe MGBR 204u |
| number | Ct | Ct | Ct | Ct |
| 1000 | 30.13 +/− 0.54 | 31.63 +/− 0.34 | 30.85 +/− 0.52 | 31.33 +/− 0.29 |
| 100 | 33.95 +/− 0.62 | 34.55 +/− 0.87 | 33.63 +/− 0.74 | 34.88 +/− 0.71 |
| 10 | 37.53 +/− 1.66 | 41.40 +/− 3.58 | 42.47 +/− 0.51 | 39.55 +/− 3.67 |
| 1 | ND | ND | ND | ND |

ND: not detected [symbol]
sensitivity is of 10 copy per PCR for all multiplex tested

TABLE 14
NG probe (ATTO 647N)

| | First | Second | Third | Fourth |
|---|---|---|---|---|
| | quadruplex mix | quadruplex mix | quadruplex mix | quadruplex mix |
| Copy | Probe MGBR140c | Probe MGBR 186j | Probe MGBR 178q | Probe MGBR 204u |
| number | Ct | Ct | Ct | Ct |
| 1000 | 26.80 +/− 0.33 | 27.13 +/− 0.40 | 27.20 +/− 0.29 | 27.28 +/− 0.22 |
| 100 | 29.85 +/− 0.10 | 30.15 +/− 0.75 | 30.20 +/− 0.64 | 30.35 +/− 0.61 |
| 10 | 32.63 +/− 1.64 | 33.20 +/− 0.43 | 32.58 +/− 0.22 | 34 +/− 0.29 |
| 1 | 36.33 +/− 0.61 | 36.15 +/− 0.13 | 36.33 +/− 1.34 | 36.53 +/− 0.39 |

ND: not detected [symbol]
Sensitivity is of 1 copy per PCR for all multiplex tested

TABLE 15
IC probe (ATTO- 590)

| | First | Second | Third | Fourth |
|---|---|---|---|---|
| | quadruplex mix | quadruplex mix | quadruplex mix | quadruplex mix |
| Copy | Probe MGBR140c | Probe MGBR 186j | Probe MGBR 178q | Probe MGBR 204u |
| number | Ct | Ct | Ct | Ct |
| 1000 | 34.38 +/− 0.17 | 35.03 +/− 0.30 | 35.25 +/− 0.64 | 34.80 +/− 0.22 |
| 100 | 34 +/− 0.18 | 34.90 +/− 0.41 | 34.75 +/− 0.13 | 34.63 +/− 0.15 |
| 10 | 34.08 +/− 0.31 | 34.38 +/− 0.28 | 34.33 +/− 0.22 | 34.80 +/− 0.20 |
| 1 | 34.55 +/− 0.13 | 34.88 +/− 0.26 | 34.65 +/− 0.31 | 35.40 +/− 0.28 |

[symbol]

There is no Ct variation for the IC, whatever quantity of DNA is being used

### 4. Example 4

### Samples Collected from Patients (First Void Urine Tests)

CT+MG+NG+IC real-time amplification systems of the invention, together in multiplex in the same mix;

compared to Roche Amplicor CT test, to in house MG PCR test, and to NG culture test.

### 4.1. Material and Methods:

### 4.1.1. Samples:

Samples are collected from human patients (Saint Louis Hospital, France). The first void urines are stored at −20° C. until use.

19 samples (1 to 19) are tested in multiplex. A negative control (sample without DNA), a positive C. trachomatis control (only C. trachomatis DNA), a positive M. genitalium control (only M. genitalium DNA) and a N. gonorrhoeae (only N. gonorrhoeae DNA) positive control are tested in the same run.

### 4.1.2. Description of the Extraction Method:

cf. Example 2

### 4.1.3. Prior Art CT or MG or NG Detection Methods:

CT detection: COBAS Amplicor® CT test available from Roche Diagnostics (Amplicor CT/NG amplification kit ref: ART: 07 59 41 4, and Cobas Amplicor CT detection kit ref.: Art 07 5749 7.)

MG detection: in-house MG PCR test: A primer set, MgPa-1 (5′-AGT TGA TGA AAC CTT AAC CCC TTG G-3′; SEQ ID NO: 55) and MgPa-3 (5′-CCG TTG AGG GGT TTT CCA TTT TTG C-3′; SEQ ID NO: 56) was used to amplify the 281 base pair fragment of the major adhesion gene (Jensen J S, Uldum S A, J Søndergård-Andersen, J Vuust, and K Lind, “Polymerase chain reaction for detection of Mycoplasma genitalium in clinical samples” J. Clin. Microbiol., 1991; 29: 46-50). The specificity of the 281 base pair amplified fragment was verified by hybridization with the 25 mer MgPa 2 probe (5′-GAC CAT CAA GGT ATT TCT CAA CAG C 3′; SEQ ID NO: 57), labelled with fluorescein-11 dUTP with use of the ECL oligonucleotide 3-tail labelling system (Amersham International, Amersham UK). The specimens from which the 281 base pair DNA fragment, visible after Southern blot hybridization with the internal probe, was obtained were regarded as positive (Casin I, Vexiau-Robert D, De La Salmoniere P, Eche A, Grandry B, Janier M. “High prevalence of Mycoplasma genitalium in the lower genitourinary tract of women attending a sexually transmitted disease clinic in Paris, France”, Sex Transm Dis., June 2002; 29: 353-359).

NG detection: standard NG culture test, as described in “Performance Standards for Antimicrobial Susceptibility Testing; sixteenth Information Supplement”, January 2006, p 130 Table 2F, edited by Clinical and Laboratory Standards Institute (CLSI) antimicrobial susceptibility testing standards M2-A9 and M7-A7.

### 4.1.4. Real-Time Multiplex PCR of the Invention:

cf. Example 2.

### Real-Time Multiplex PCR

Composition of the Mix

In a 1×PCR mix, add the following primers:

CT forward primer U-PC 5580 (SEQ ID NO: 6) at 0.125 μM,

CT reverse primer L-PC 5754 (SEQ ID NO: 12) at 0.5 μM,

MG forward primer U-MG 1320 (SEQ ID NO: 15) at 0.025 μM,

MG reverse primer L-MG 1578 (SEQ ID NO: 18) at 1.25 μM,

NG forward primer U-pilE 159 (SEQ ID NO: 44) at 0.85 μM,

NG reverse primer L-pilE 406 (SEQ ID NO: 47) at 0.25 μM,

IC forward primer IS 368 (SEQ ID NO: 49) at 0.5 μM, and

IC reverse primer IS 569 (SEQ ID NO: 52) at 0.5 μM.

The probes are used at the following concentrations:

CT probe SCT 175b (SEQ ID NO: 9) at 0.15 μM (fluorophore FAM),

MG probe SFMG 258c (SEQ ID NO: 17) at 0.2 μM (fluorophore TAMRA), and

NG probe pilEc (SEQ ID NO: 46) at 0.2 μM (fluorophore ATTO 647N).

IC probe SIMB (SEQ ID NO: 51) at 0.15 μM (fluorophore ATTO-590),

The following reactants are further added:

5% Glycerol, 0.3% PVP 10, 2 U of Taq Polymerase, 1 mM dNTP and 5 mM final of

MgCl₂.

10 μL of DNA are added to this mix.

Thermocycling:

First cycle: 15″ at 95° C.

Second cycle: 30″ at 95° C.

Third cycle: 50″ at 58° C.

Fourth cycle: 30″ at 72° C.

Repeat 50 times from cycle 2 to cycle 4

TABLE 16
4.2. Results:

| | First PCR | Second | Culture | Real Time PCR, Present invention | | | | | | | |
|---|---|---|---|---|---|---|---|---|---|---|---|
| | result | PCR result | result | C. trachomatis | | M. genitalium | | N. gonorrhoeae | | IC | |
| Sample | C. trachomatis | M. genitalium | N. gonorrhoeae | Mean | SD | Mean | SD | Mean | SD | Mean | SD |
| 1 | + | − | − | 33.0 | 00.0 | ND | ND | ND | ND | 34.15 | 0.35 |
| 2 | + | − | − | 35.95 | 0.07 | ND | ND | ND | ND | 34.70 | 0.14 |
| 3 | + | − | − | 27.95 | 0.21 | ND | ND | ND | ND | 34.70 | 0 |
| 4 | + | − | − | 23.95 | 0.21 | ND | ND | ND | ND | 34.10 | 0 |
| 5 | + | − | − | 31.70 | 0.14 | ND | ND | ND | ND | 34.25 | 0.21 |
| 6 | + | − | − | 33.75 | 0.35 | ND | ND | ND | ND | 34.75 | 0.07 |
| 7 | + | − | − | 29.05 | 0.07 | ND | ND | ND | ND | 34.65 | 0.07 |
| 8 | + | − | − | 29.95 | 0.07 | ND | ND | ND | ND | 34.05 | 0.19 |
| 9 | + | − | − | 24.50 | 0.28 | ND | ND | ND | ND | 34.95 | 0.07 |
| 10 | + | − | − | 31.25 | 0.49 | ND | ND | ND | ND | 34.80 | 0.57 |
| 11 | − | − | + | ND | ND | ND | ND | 37.05 | 1.34 | 35.15 | 0.92 |
| 12 | + | − | − | 31 | 0 | ND | ND | ND | ND | 34.10 | 0.28 |
| 13 | − | − | + | ND | ND | ND | ND | 45.15 | 3.04 | 34.60 | 0.14 |
| 14 | − | − | + | ND | ND | ND | ND | 29.50 | 0.28 | 35.45 | 0.92 |
| 15 | + | − | − | 31.80 | 0.42 | ND | ND | ND | ND | 34.55 | 0.35 |
| 16 | + | − | − | 32.75 | 0.07 | ND | ND | ND | ND | 35.30 | 0.57 |
| 17 | − | − | + | ND | ND | ND | ND | 33.45 | 0.21 | 34.75 | 0.07 |
| 18 | − | − | + | ND | ND | ND | ND | 39.30 | 0.85 | 34.80 | 0.42 |
| 19 | + | − | − | 31.20 | 0.42 | ND | ND | ND | ND | 34.45 | 0.92 |
| Negative | NT | NT | NT | ND | ND | ND | ND | ND | ND | 34.25 | 0.07 |
| control | | | | | | | | | | | |
| Positive CT | NT | NT | NT | 28.85 | 0.21 | ND | ND | ND | ND | 35.40 | 0.14 |
| control | | | | | | | | | | | |
| Positive | NT | NT | NT | ND | ND | 31.80 | 0.14 | ND | ND | 34.65 | 0.21 |
| M. genitalium | | | | | | | | | | | |
| control | | | | | | | | | | | |
| Positive | NT | NT | NT | ND | ND | ND | ND | 25.30 | 0.14 | 35.60 | 0.57 |
| N. gonorrhoeae | | | | | | | | | | | |
| control | | | | | | | | | | | |

(ND: not detected, SD: Standard deviation, IC: internal control)

The IC is perfectly detected in the real-time multiplex system of the invention, which confirms that there is no Taq polymerase inhibitor.

The real-time multiplex amplification of the invention has a good reproducibility, the standard deviations being very low, except for two points on N. gonorrhoeae.

The detection results obtained with the real-time multiplex system of the invention are at least as accurate as the ones obtained with the prior art ones.

I claim:

1. A process for the detection of at least two different bacterial species selected from the group consisting of Chlamydia trachomatis (CT), Mycoplasma genitalium (MG) and Neisseria gonorrhoeae (NG), in a sample, wherein said process is a real-time multiplex amplification process, wherein said detection comprises the determination of whether at least one amplicon has been or is produced from said sample, or from nucleic acid material thereof, by amplification by means of amplification primers and real-time probes, whereby a positive determination indicates that said bacterial species is(are) present in said sample, wherein said amplification primers and real-time probes comprise at least two of the following three i.-iii. elements:
i. at least two primers intended for targeting CT, which are oligonucleotides consisting of 14-30 nucleotides, the sequences of which are suitable for use as forward and reverse primers, respectively, in the amplification of at least one CT template sequence, and at least one real-time probe intended for targeting CT, wherein the sequence of said at least one CT-targeted real-time probe sequence comprises the sequence of a fragment of at least 15 nucleotides of said at least one CT template sequence or the complementary sequence thereof or a sequence that is at least 90% identical to said fragment or complementary sequence, and wherein said at least one CT template sequence is SEQ ID NO: 5 or SEQ ID NO: 6,
ii. at least two primers intended for targeting MG, which are oligonucleotides consisting of 14-30 nucleotides, the sequences of which are suitable for use as forward and reverse primers, respectively, in the amplification of at least one MG template sequence, and at least one real-time probe intended for targeting MG, wherein the sequence of said at least one MG-targeted real-time probe sequence comprises the sequence of a fragment of at least 15 nucleotides of said at least one MG template sequence or the complementary sequence thereof or a sequence that is at least 90% identical to said fragment or complementary sequence, and wherein said at least one MG template sequence is SEQ ID NO: 13 or SEQ ID NO: 14 or SEQ ID NO: 19 or SEQ ID NO: 20 or SEQ ID NO: 25 or SEQ ID NO: 26 or SEQ ID NO: 31 or SEQ ID NO: 32 or SEQ ID NO: 37 or SEQ ID NO: 38,
iii. at least two primers intended for targeting NG, which are oligonucleotides consisting of 14-30 nucleotides, the sequences of which are suitable for use as forward and reverse primers, respectively, in the amplification of at least one NG template sequence, and at least one real-time probe intended for targeting NG, wherein the sequence of said at least one NG-targeted real-time probe sequence comprises the sequence of a fragment of at least 15 nucleotides of said at least one NG template sequence or the complementary sequence thereof or a sequence that is at least 90% identical to said fragment or complementary sequence, and wherein said at least one NG template sequence is SEQ ID NO: 42 or SEQ ID NO: 43.

2. The detection process of claim 1, wherein said at least two CT-targeted primers are the oligonucleotides of SEQ ID NOs: 7 and 12 respectively.

3. The detection process of claim 1, wherein said at least two MG-targeted primers are the oligonucleotides of SEQ ID NOs: 15 and 18 respectively, or the oligonucleotides of SEQ ID NOs: 21 and 24 respectively, or the oligonucleotides of SEQ ID NOs: 27 and 30 respectively, or the oligonucleotides of SEQ ID NOs: 33 and 36 respectively, or the oligonucleotides of SEQ ID NOs: 39 and 36 respectively.

4. The detection process of claim 1, wherein said at least two NG-targeted primers are the oligonucleotides of SEQ ID NOs: 44 and 47 respectively.

5. The detection process of claim 1, wherein said at least one CT-targeted real-time probe comprises the sequence of SEQ ID NO: 8 or the complementary sequence thereof, or the sequence of SEQ ID NO: 10 or the complementary sequence thereof.

6. The detection process of claim 1, wherein said at least one CT-targeted real-time probe comprises a fragment of 22-27 nucleotides of said at least one CT template sequence or the complementary sequence thereof, or wherein said MG-targeted real-time probe comprises a fragment of 22-27 nucleotides of said at least one MG template sequence or the complementary sequence thereof, or wherein said NG-targeted real-time probe comprises a fragment of 22-27 nucleotides of said at least one NG template sequence or the complementary sequence thereof.

7. The detection process of claim 1, wherein said at least one MG-targeted real-time probe comprises the sequence of SEQ ID NO: 16 or the complementary sequence thereof, or the sequence of SEQ ID NO: 22 or the complementary sequence thereof, or the sequence of SEQ ID NO: 28 or the complementary sequence thereof, or the sequence of SEQ ID NO: 34 or the complementary sequence thereof, or the sequence of SEQ ID NO: 40 or the complementary sequence thereof.

8. The detection process of claim 1, wherein said at least one NG-targeted real-time probe comprises the sequence of SEQ ID NO: 45 or the complementary sequence thereof.

9. The detection process of claim 1, wherein said at least one CT-targeted real-time probe further comprises an oligonucleotide of 3-10 nucleotides linked to the 5′ end of said CT-specific probe sequence and an oligonucleotide of 3-10 nucleotides linked to the 3′ end of said CT-specific probe sequence, wherein said 5′ end oligonucleotide and 3′ end oligonucleotide impart a hairpin configuration to said at least one real-time CT-specific probe, when said at least one real-time CT-specific probe is unhybridized, or wherein said at least one MG-targeted real-time probe further comprises an oligonucleotide of 3-10 nucleotides linked to the 5′ end of said MG-specific probe sequence and an oligonucleotide of 3-10 nucleotides linked to the 3′ end of said MG-specific probe sequence, wherein said 5′ end oligonucleotide and 3′ end oligonucleotide impart a hairpin configuration to said at least one real-time MG-specific probe, when said at least one real-time MG-specific probe is unhybridized, or wherein said at least one NG-targeted real-time probe further comprises an oligonucleotide of 3-10 nucleotides linked to the 5′ end of said NG-specific probe sequence and an oligonucleotide of 3-10 nucleotides linked to the 3′ end of said NG-specific probe sequence, wherein said 5′ end oligonucleotide and 3′ end oligonucleotide impart a hairpin configuration to said at least one real-time NG-specific probe, when said at least one real-time NG-specific probe is unhybridized.

10. The detection process of claim 9, wherein the sequence of said at least one CT-targeted real-time probe is SEQ ID NO: 9 or the complementary sequence thereof, or SEQ ID NO: 11 or the complementary sequence thereof, or wherein the sequence of said at least one MG-targeted real-time probe is SEQ ID NO: 17 or the complementary sequence thereof, or SEQ ID NO: 23 or the complementary sequence thereof, or SEQ ID NO: 29 or the complementary sequence thereof, or SEQ ID NO: 35 or the complementary sequence thereof, or SEQ ID NO: 41 or the complementary sequence thereof, or wherein the sequence of said at least one NG-targeted real-time probe is SEQ ID NO: 46 or the complementary sequence thereof.

11. The detection process of claim 1, wherein said amplification primers and said real-time probes comprise said at least two CT-targeted primers and said at least one CT-targeted real-time probe of i., and said at least two MG-targeted primers and said at least one MG-targeted real-time probe of ii., and said at least two NG-targeted primers and said at least one NG-targeted real-time probe of iii.

12. A process of production of primers and real-time probes, which comprises producing a first oligonucleotide, a second oligonucleotide, a third oligonucleotide, a fourth oligonucleotide, a fifth oligonucleotide, a sixth oligonucleotide, a seventh oligonucleotide, a eighth oligonucleotide and a ninth oligonucleotide, wherein
said first oligonucleotide is a primer of 14-30 nucleotides, the sequence of which is at least 80% identical to the sequence of the same length that is the 5′ terminal end of a template sequence,
said second oligonucleotide is a primer of 14-30 nucleotides, the sequence of which is at least 80% identical to the sequence of the same length that is the 5′ terminal end of the sequence that is complementary to said template sequence, and
said third oligonucleotide is a real-time probe, which comprises the sequence of a fragment of at least 15 nucleotides of said template sequence or of the complementary sequence thereof, or a sequence, which is at least 90% identical to the sequence of said fragment or complementary sequence,
wherein, for said first, second and third oligonucleotides, said template sequence is the same sequence selected from the CT group consisting of SEQ ID NO: 5 and SEQ ID NO: 6,
wherein said first and second oligonucleotides form a primer pair that targets CT and said third oligonucleotide is a real-time probe that targets CT;
wherein
said fourth oligonucleotide is a primer of 14-30 nucleotides, the sequence of which is at least 80% identical to the sequence of the same length that is the 5′ terminal end of a template sequence,
said fifth oligonucleotide is a primer of 14-30 nucleotides, the sequence of which is at least 80% identical to the sequence of the same length that is the 5′ terminal end of the sequence that is complementary to said template sequence, and
said sixth oligonucleotide a real-time probe, which comprises the sequence of a fragment of at least 15 nucleotides of said template sequence or the complementary sequence thereof or a sequence, which is at least 90% identical to said fragment or complementary sequence,
wherein, for said fourth, fifth and sixth oligonucleotides, said template sequence is the same sequence selected from the MG group consisting of SEQ ID NO: 13, SEQ ID NO: 14, SEQ ID NO: 19, SEQ ID NO: 20, SEQ ID NO: 25, SEQ ID NO: 26, SEQ ID NO: 31, SEQ ID NO: 32, SEQ ID NO: 37 and SEQ ID NO: 38,
wherein said fourth and fifth oligonucleotides form a primer pair that targets MG and said sixth oligonucleotide is a real-time probe that targets MG;
wherein
said seventh oligonucleotide is a primer of 14-30 nucleotides, the sequence of which is at least 80% identical to the sequence of the same length that is the 5′ terminal end of a template sequence,
said eighth oligonucleotide is a primer of 14-30 nucleotides, the sequence of which is at least 80% identical to the sequence of the same length that is the 5′ terminal end of the sequence that is complementary to said template sequence, and
said ninth oligonucleotide a real-time probe, which comprises the sequence of a fragment of at least 15 nucleotides of said template sequence or the complementary sequence thereof or a sequence, which is at least 90% identical to said fragment or complementary sequence,
wherein, for said seventh, eighth and ninth oligonucleotides, said template sequence is the same sequence selected from the NG group consisting of SEQ ID NO: 42 and SEQ ID NO: 43,
wherein said seventh and eighth oligonucleotides form a primer pair that targets NG and said ninth oligonucleotide is a real-time probe that targets NG,
and wherein said first, second, third, fourth, fifth, sixth, seventh, eighth and ninth oligonucleotides are suitable for use in mixture for the detection of Chlamydia trachomatis (CT), Mycoplasma genitalium (MG) and Neisseria gonorrhoeae (NG) by real-time amplification.

13. The process of claim 12, wherein each of said third, sixth and ninth oligonucleotides is linked to a reporter dye.

14. The process of claim 12, which further comprises linking a quencher at one of the ends of each of said third, sixth and ninth oligonucleotides.

15. The process of claim 12, wherein said third oligonucleotide further comprises an oligonucleotide of 3-10 nucleotides linked to its 5′ end and an oligonucleotide of 3-10 nucleotides linked to its 3′ end, wherein said 5′ end oligonucleotide and 3′ end oligonucleotide impart a hairpin configuration to said third oligonucleotide, when said third oligonucleotide is unhybridized, wherein said sixth oligonucleotide further comprises an oligonucleotide of 3-10 nucleotides linked to its 5′ end and an oligonucleotide of 3-10 nucleotides linked to its 3′ end, wherein said 5′ end oligonucleotide and 3′ end oligonucleotide impart a hairpin configuration to said sixth oligonucleotide, when said sixth oligonucleotide is unhybridized, and wherein wherein said ninth oligonucleotide further comprises an oligonucleotide of 3-10 nucleotides linked to its 5′ end and an oligonucleotide of 3-10 nucleotides linked to its 3′ end, wherein said 5′ end oligonucleotide and 3′ end oligonucleotide impart a hairpin configuration to said ninth oligonucleotide, when said ninth oligonucleotide is unhybridized.

16. The process of claim 12, wherein the sequences of said first and second oligonucleotides are SEQ ID NOs: 7 and 12 respectively, and the sequence of said third oligonucleotide is SEQ ID NO: 9 or SEQ ID NO: 11 or one of the complementary sequences thereof or comprises SEQ ID NO: 8 or SEQ ID NO: 10 or one of the complementary sequences thereof, wherein
the sequences of said fourth and fifth oligonucleotides are SEQ ID NOs: 15 and 18 respectively, and the sequence of said sixth oligonucleotide is SEQ ID NO: 17 or the complementary sequence thereof or comprises SEQ ID NO: 16 or the complementary sequence thereof,
or
the sequences of said fourth and fifth oligonucleotides are SEQ ID NOs: 21 and 24 respectively, and the sequence of said sixth oligonucleotide is SEQ ID NO: 23 or the complementary sequence thereof or comprises SEQ ID NO: 22 or the complementary sequence thereof,
or
the sequences of said fourth and fifth oligonucleotides are SEQ ID NOs: 27 and 30 respectively, and the sequence of said sixth oligonucleotide is SEQ ID NO: 29 or the complementary sequence thereof or comprises SEQ ID NO: 28 or the complementary sequence thereof,
or
the sequences of said fourth and fifth oligonucleotides are SEQ ID NOs: 33 and 36 respectively, and the sequence of said sixth oligonucleotide is SEQ ID NO: 35 or the complementary sequence thereof or comprises SEQ ID NO: 34 or the complementary sequence thereof,
or
the sequences of said fourth and fifth oligonucleotides are SEQ ID NOs: 39 and 36 respectively, and the sequence of said sixth oligonucleotide is SEQ ID NO: 41 or the complementary sequence thereof or comprises SEQ ID NO: 40 or the complementary sequence thereof,
and wherein the sequences of said seventh and eighth oligonucleotides are SEQ ID NOs: 44 and 47 respectively, and the sequence of said ninth oligonucleotide is SEQ ID NO: 46 or the complementary sequence thereof or comprises SEQ ID NO: 45 or the complementary sequence thereof.

17. The process of claim 12, wherein said first, second, third, fourth, fifth, sixth, seventh, eighth and ninth oligonucleotides are a set of primers and real-time probes, which does not detect Escherichia coli, Enterobacter cloacae, Staphylococcus saprophyticus, Enterococcus faecium, Enterococcus faecalis, Proteus mirabilis, Bacillus subtilis, Gardnerella vaginalis, Pseudomonas aeruginosa, Strepcococcus agalactiae, Acinetobacter baumanii, Staphylococcus epidermidis and Moraxella catarrhalis by real-time amplification.

18. The process of claim 12, wherein said first, second, third, fourth, fifth, sixth, seventh, eighth and ninth oligonucleotides are a set of primers and real-time probes, which does not detect Escherichia coli, Enterobacter cloacae, Staphylococcus saprophyticus, Enterococcus faecium, Enterococcus faecalis, Proteus mirabilis, Lactobacillus acidophilus, Bacillus subtilis, Gardnerella vaginalis, Neisseria gonorrhoeae, Staphylococcus aureus, Pseudomonas aeruginosa, Klebsiella pneumoniae, Strepcococcus agalactiae, Streptococcus bovis, Acinetobacter baumanii, Staphylococcus epidermidis, Candida albicans, Mycoplasma pneumoniae, Neisseria mucosa, Rhodococcus equi, Moraxella catarrhalis, Mycoplasma orale, Mycoplasma fermentans and Mycoplasma penetrans by real-time amplification.

19. The process of claim 12, wherein said first, second, third, fourth, fifth, sixth, seventh, eighth and ninth oligonucleotides are a set of primers and real-time probes, which detects Chlamydia trachomatis serovars A, B, Ba, C, D, E, F, H, I, J, K, L1, L2a and L3 by real-time amplification.

20. A process of production of primers and real-time probes suitable for the detection by real-time amplification of at least two bacterial species selected from the group consisting of Chlamydia trachomatis (CT), Mycoplasma genitalium (MG) and Neisseria gonorrhoeae (NG), which comprises producing a first oligonucleotide, a second oligonucleotide, a third oligonucleotide, a fourth oligonucleotide, a fifth oligonucleotide and a sixth oligonucleotide, wherein
said first oligonucleotide is a primer of 14-30 nucleotides, the sequence of which is at least 80% identical to the sequence of the same length that is the 5′ terminal end of a template sequence,
said second oligonucleotide is a primer of 14-30 nucleotides, the sequence of which is at least 80% identical to the sequence of the same length that is the 5′ terminal end of the sequence that is complementary to said template sequence,
said third oligonucleotide is a real-time probe, which comprises the sequence of a fragment of at least 15 nucleotides of said template sequence or the complementary sequence thereof or a sequence, which is at least 90% identical to the sequence of said fragment or complementary sequence,
wherein, for said first, second and third oligonucleotides, said template sequence is the same sequence selected from one of the following three groups:
the CT group consisting of SEQ ID NO: 5 and SEQ ID NO: 6;
the MG group consisting of SEQ ID NO: 13, SEQ ID NO: 14, SEQ ID NO: 19, SEQ ID NO: 20, SEQ ID NO: 25, SEQ ID NO: 26, SEQ ID NO: 31, SEQ ID NO: 32, SEQ ID NO: 37 and SEQ ID NO: 38; and
the NG group consisting of SEQ ID NO: 42 and SEQ ID NO: 43,
wherein said first and second oligonucleotides form a primer pair that targets the same CT, MG or NG bacterial species as the group from which said template sequence has been selected, and said third oligonucleotide is a real-time probe that targets the same CT, MG or NG bacterial species as the group from which said template sequence has been selected;
wherein
said fourth oligonucleotide is a primer of 14-30 nucleotides, the sequence of which is at least 80% identical to the sequence of the same length that is the 5′ terminal end of a template sequence,
said fifth oligonucleotide is a primer of 14-30 nucleotides, the sequence of which is at least 80% identical to the sequence of the same length that is the 5′ terminal end of the sequence that is complementary to said template sequence, and
said sixth oligonucleotide is a real-time probe, which comprises the sequence of a fragment of at least 15 nucleotides of said template sequence or the complementary sequence thereof, or a sequence, which is at least 90% identical to the sequence of said fragment or complementary sequence,
wherein, for said fourth, fifth and sixth oligonucleotides, said template sequence is the same sequence selected from one of said CT-specific, MG-specific and NG-specific groups,
wherein said fourth and fifth oligonucleotides form a primer pair that targets the same CT, MG or NG bacterial species as the group from which said template sequence has been selected, and said sixth oligonucleotide is a real-time probe that targets the same CT, MG or NG bacterial species as the group from which said template sequence has been selected;
wherein the group selected for said first, second and third oligonucleotides is different from the group selected for said fourth, fifth and sixth oligonucleotides, and
wherein said first, second, third, fourth, fifth and sixth oligonucleotides are suitable for use in mixture for the detection of Chlamydia trachomatis (CT), Mycoplasma genitalium (MG) and Neisseria gonorrhoeae (NG) by real-time amplification.
